(19) United States
(12) Patent Application Publication
(10) Pub. No.: US 2022/0073980 A1
(43) Pub. Date: Mar. 10, 2022

(54) SEQUENCING BY COALESCENCE

(71) Applicant: XGenomes Corp., Cambridge, MA (US)

(72) Inventor: Kalim Mir, Cambridge, MA (US)

(21) Appl. No.: 17/298,487

(22) PCT Filed: Nov. 27, 2019
(86) PCT No.: PCT/US19/63551

§ 371 Date: May 28, 2021

Related U.S. Application Data
(60) Provisional application No. 62/772,979, filed on Nov. 29, 2018.

(51) Int. Cl. C12Q 1/6874; G16B 30/20; G16B 40/10

(52) U.S. Cl. CPC C12Q 1/6874; G16B 40/10; G16B 30/20

(57) ABSTRACT

A method of sequencing a single, elongated target polynucleotide molecule can include the steps of seeding a plurality of separately resolvable origins of polynucleotide synthesis along the single, elongated target polynucleotide; contacting the target polynucleotide with a polymerase and labelled nucleotides; incorporating a labelled nucleotide, using the polymerase, into a plurality of sequence fragments complementary to the target polynucleotide and originating from the origins of polynucleotide synthesis; identifying and storing the identity and positions of the labelled nucleotide incorporated into each of the plurality of sequence fragments; and repeating the incorporating and identifying steps until adjacent sequence fragments coalesce and result in continuous sequence reads spanning two or more adjacent sequence fragments.

### BACKGROUND OF THE INVENTION

Sequencing the human genome for the first time took more than ten years and hundreds of millions of dollars. Historically there had been two successful approaches to DNA sequence determination: the dideoxy chain termination method, e.g., Sanger et al, Proc. Natl. Acad. Sci., 74:5463-5467 (1977); and the chemical degradation method, e.g. Maxam et al, Proc. Natl. Acad. Sci., 74:560-564 (1977). These methods of sequencing nucleotides were both time consuming and expensive.

Sanger dideoxy sequencing provides sequence information rather indirectly, by looking at the differences in gel-migration of a ladder of terminated extension reactions. Nevertheless this basic approach, when automated, run in capillaries and with fluorescently labeled nucleotides provided the means to sequence the consensus human genome. The gel electrophoretic separation step, which is labor intensive, is difficult to automate, and introduces an extra degree of variability in the analysis of data, e.g. band broadening due to temperature effects, compressions due to secondary structure in the DNA sequencing fragments, in-homogeneities in the separation gel. However, the need for large-scale sequencing of individual human genomes, the genomes of other organisms and pathogens required lower-cost and more rapid alternatives to be developed (Mir, KU. Sequencing Genomes: From Individuals to Populations, Briefings in Functional Genomics and Proteomics, 8: 367-378 (2009). Several methods that avoid gel electrophoresis have been developed as “next generation sequencing”.

The detection methods used in the most evolved form of Sanger sequencing and the currently dominant Illumina technology is fluorescence. Other detection means include detection using a proton release via Field Effect Transistor, an ionic current through a nanopore and electron microscopy.

Methods have been explored in which the concept of determining sequence information by cleaving bases or by template directed synthesis is implemented in ways that avoid gel electrophoresis. Sequencing by exonuclease digestion of individual nucleotide from single DNA molecules is one of the oldest of these approaches (CA1314247). The opposite approach of adding to a primer includes Sequencing by Ligation (Shendure et al Science 309:1728-1732(2005)) which interrogates the sequence within an oligonucleotide (oligo) footprint adjacent to a primer and includes “sequencing by synthesis” (SbS) which can be conducted by ligation (Mir et al Nucleic Acids Research 37: e5 2009, SOLID) or polymerase extension. SbS via polymerase has become the dominant next generation technology and is described, for example, in U.S. Pat. No. 5,302,509. It involves the identification of each nucleotide immediately following its incorporation or while it is being incorporated by a polymerase into an extending DNA strand. One SbS approach, pyrosequencing [Ronaghi M, Uhlen M, Nyren P. A sequencing method based on real-time pyrophosphate. Science. 1998 Jul 17;281(5375):363,36], has been used for SNP (single-nucleotide polymorphism) typing and DNA sequencing as part of 454 sequencing. In this case, the detection is bioluminescent based on pyrophosphate (PPi) release, its conversion to ATP by ATP sulfurase, and the consumption of the ATP by firefly luciferase in the production of visible light (luminescence) without needing to excite a fluorophore. However, because the signal is diffusible, pyrosequencing cannot take advantage of the massive degree of parallelism that becomes available when surface immobilized reactions are analyzed. In one embodiment the Luciferase is immobilized in the vicinity of the incorporation reaction and in some embodiments the ATP sulfurase is also immobilized in the same vicinity, enabling the luminescence generation to be localized. It also adds only one of the four nucleotides, A,C,G or T at a time and also struggles to determine the numbers of bases when there is a homopolymer run in the target. Ion Torrent conducts sequencing in essentially the same way but electrically detects the liberation of a proton by a chemFET rather than the liberation of PPi via luciferase luminescence. The dominant SbS approach is cyclical sequencing using reversible terminators (Metzker Nucleic Acids Research 22:4259-4267 (1994)) which has been successfully commercialized by Illumina (Bentley et al., Nature 456:53-59 (2008)) and is the dominant sequencing technology today.

Illumina sequencing starts with single genomic molecules which are clonally amplified. Substantial upfront sample processing is needed to convert the target genome into a library which is then clonally amplified as clusters. The other technology which is capable of routinely sequencing whole genomes is a ligation sequencing method conducted on clonally amplified templates (DNA nanoballs) which are isolated in an array of wells (Complete Genomics) (Drmanac et al Science 237:78-81 (2010).

However, methods have reached the market that have circumnavigated the need for amplification and conducted fluorescent SbS on single molecules of DNA. The first method is from HelicosBio (now SeqLL), and conducts stepwise SbS with reversible termination (Harris et al). The second method from Pacific Biosciences uses labels on a terminal phosphate, a natural leaving group of the incorporation reaction, which allows sequencing to be conducted continuously, without the need for exchanging reagents; one of the downsides of this approach is that throughput is low as the detector needs to remain fixed on one field of view Levene et al. Science 299, 682-686 (2003); Eid et al, Science, 323:133-8 (2009). A somewhat similar approach to Pacific Bioscience sequencing is the method being developed by Genia (now part of Roche) which detects SbS via a nanopore, rather than optical methods. Further, Oxford Nanopore Technologies have a nanopore approach which has demonsatrated read lengths of close to a million bases but its error rate is hifg. Finally, sequencing methods using transmission electron microscopy to directly spatially detect the identity of individual labeled nucleotides on stretched DNA has been investigated by companies such as ZS Genetics and Halcyon Molecular but have yet to lead to a working sequencing technology.

The human genome is organized over 46 chromosomes, of which the shortest is about 50 megabases and the longest 250 megabases. But the read lengths obtained by Sanger sequencing are in the 1000 base range, 454 and Ion Torrent in the several 100s of bases range and Illumina sequencing which is initially started with a read of about 25 bases is now an order of magnitude longer. However, as fresh reagents need to be supplied per base of the read length, sequencing 250 bases rather than 25 requires 10× more time and 10× more of the costly reagents. Recently, the standard read-lengths of Illumina instruments have been decreased to around 150 bases, presumably due to their technology being subject to phasing (molecules within clusters getting out of synchronization) which introduces error as the reads get longer.

The longest read lengths in commercial systems are obtained by nanopores strand sequencing and Pacific Bioscience sequencing, the latter of which has reads that average 10,000 bases in length. Whilst these longer read lengths are desirable they come at the cost of accuracy. Accuracy is so poor that for most applications these methods can only be used as a supplement to Illumina sequencing, not as a sequencing technology in their own right. Moreover, the throughput of existing long-read technologies is too low for routine human genome scale sequencing.

Besides ONT and PacBio sequencing, a number of approaches exist that are not sequencing technologies per se, rather sample preparation approaches that supplement Ilumina short read sequencing technology to provide a scaffold for building longer reads. Of these, two deserve mention, the first is the droplet based technology developed by 10X Genomics, which isolates 100-200 kb fragments (the average length range of fragments after extraction) within droplets and process them into libraries of shorter length fragments each of which contains a sequence identifiers tag specific for the 100-200 kb from which they originate, which upon sequencing of the genome from a multiplicity of droplets can be deconvolved into ˜50-200 Kb buckets. The second is an approach developed by Bio Nanogenomcs which stretches DNA and fluorescently detects points of nicking induced by a nicking endounclease, to provide a map or scaffold, which at present is not high enough density to help assemble genomes, but nevertheless provides a direct visualization of the genome and is able to detect large structural variations and determine long-range haplotypes.

Mate pair libraries and paired-end sequencing enables some long-range information to be gathered. Helicos Inc. proposed paired reads, with known distances between reads obtained on single molecules, one after the other. What paired reads are able to detect is whether a divergence from a reference exists. Due to structural variation two sites may not be linked as expected, or may be unexpectedly are linked. What paired reads do not tell you is the overall architecture of the genome. For example, if a first sequence that was expected to be linked to a second is not there, is it deleted? Has an intervening insertion or deletion changed the relative distance between two sequences? Has the sequence moved to somewhere else in the genome? With linking of just two reads these questions cannot be easily answered.

Mir (WO2005040425) and Ramanathan A et al Anal Biochem. 2004 July 15;330(2):227-41 described starting sequencing sites along single DNA molecules. Ramanathan et al have show extension from a nick and gapped template, when a single correct nucleotide is added after photobleaching of the fluorochrome they have sown second base extension by adding the correct nucleotide only, labeled with the first fluorochrome as the first. However, it is not evident from the data that the second extension is from the same location as the first nucleotide, as there can be significant difference in location between the signals. The polynucleotides are not linearly aligned in a single orientation. Therefore there is no evidence that two contiguous nucleotides have been added on a single polynucleotide, to generate a 2 base read. In addition 30% of cycles had two additions and 70% had one addition. In second cycles 45% had two additions, but 20% had three; the authors acknowledge that the apparent two additions could actually be due to signal from two separate molecules due to DNA not remaining stuck to the surface. It is known from the experience of 454 and Ion Torrent sequencing that non-terminated nucleotide addition introduces errors due to a difficulty in determining the number of nucleotides added to properly read a homopolymer region. Moreover, if more than one fluorescently labeled nucleotide is incorporated in one cycle, consecutive fluorochromes will be separated by sub-nm to a few nm range (depending on the linker used) and are likely to interfere with each other's readout, for example by quenching, energy transfer, or by obfuscating the order of the bases. The paper shows some basic concepts but does not show how to construct a working system: how a full read can be obtained; how a set of reads can be coalesced; or how the reads obtained from multiple molecules integrated to provide a genome assembly.

Jerrod Schwartz et al PNAS 2012;109:18749-18754 have elongated template DNA and attempted to perform cluster amplification along their length but the results are poor, with less than 0.5% of reads showing any semblance of being paired.

Therefore there remains need for a stand-alone (e.g. without requiring supplementary technology) sequencing technologies that are efficient in the use of reagents and time and can provide long, haplotype resolved, persistent (can go through repetitive regions etc) read-lengths without sacrificing accuracy.

### BRIEF SUMMARY OF THE INVENTION

In the present invention we describe methods that can start sequencing synthesis reads directly on native polynucleotides such as genomic DNA, and the invention teaches how these reads can be made in a way that covers the whole polynucleotide or assembles a complete polynucleotide (e.g. chromosome) due to coalescence of reads. In some embodiments, the native polynucleotides require no processing before they are displayed for sequencing. This allows the method to also integrate epigenomic information as the chemical modifications of DNA will stay in place. The polynucleotides are directionally well aligned and therefore relatively easy to image, image process base call and assemble; the sequence error rate is low and coverage is high. A number of ways of carrying out the invention are described, at both bulk and single molecule level but each is done so that the burden of sample preparation is wholly or almost wholly eliminated.

The invention is surprising and counter-intuitive because it allows a million or more contiguous bases of genomic DNA to be sequenced by carrying out less than a hundred sequencing cycles. The invention is based, in part, on the discovery that single, elongated target polynucleotide molecules can be sequenced from multiple origins of synthesis that coalesce into continuous sequence reads.

Accordingly, the invention, in various aspects and embodiments includes: obtaining long lengths of polynucleotides; disposing the polynucleotide in a linear state such that locations along its length can be traced; creating multiple sites (origins) along the polynucleotide length so that each site has a site positioned upstream and a site positioned downstream of itself (with the exception of the two sites closes to each of the ends of the polynucleotide) and which can prime template directed DNA synthesis for example, by nicking to create a 3′ end or annealing an oligo containing a 3′ end; extending each of the 3′ ends (fronts), as growing chains, in template-directed reactions, with the strand to be sequenced as the template, using a polymerase to incorporate a nucleotide complementary to the nucleotide present in each of the multiple sites in the target strand; detecting the identity of the incorporated nucleotide at each of the multiple sites; incorporating the next nucleotide complementary to each of the multiple sites and detecting the identity of the incorporated nucleotide; repeating incorporation and detection at each of the multiple sites so that the front of synthesis at each of the multiple sites migrates along the target polynucleotide in a 5′ to 3′ direction until a threshold number of fronts reach downstream origins.

The invention, in various aspects and embodiments also includes a method of sequencing a target polynucleotide molecule comprising: (a) seeding a plurality of separately resolvable origins of polynucleotide synthesis along each of a plurality of copies of the target polynucleotide molecule; (b) contacting the plurality of copies with a polymerase and four types of differently labelled nucleotides simultaneously; (c) incorporating the differently labelled nucleotides, using the polymerase, into a plurality of sequence fragments complementary to the target polynucleotide molecule and originating from the origins of polynucleotide synthesis; (d) identifying and storing the identity and positions of the differently labelled nucleotides incorporated into each of the plurality of sequence fragments, thereby determining the sequences and relative positions of the plurality of sequence fragments; (e) repeating steps (c) and (d) until a threshold number of nucleotides are sequenced; and (f) assembling the plurality of sequence fragments, thereby determining the sequence of the elongated, target polynucleotide molecule.

The invention, in various aspects and embodiments also includes a method of sequencing a single, elongated target polynucleotide molecule comprising: (a) seeding a plurality of separately resolvable origins of polynucleotide synthesis along the target polynucleotide molecule;

(b) contacting the target polynucleotide molecule with a polymerase and four types of differently labelled nucleotides simultaneously; (c) incorporating the differently labelled nucleotides, using the polymerase, into a plurality of sequence fragments complementary to the target polynucleotide molecule and originating from the origins of polynucleotide synthesis; (d) identifying and storing the identity and positions of the differently labelled nucleotides incorporated into each of the plurality of sequence fragments, thereby determining the sequences and relative positions of the plurality of sequence fragments; and (e) repeating steps (c) and (d) until a threshold number of nucleotides are sequenced; and (f) comparing the sequences and relative positions of the plurality of sequence fragments to a reference sequence for the target polynucleotide molecule, thereby ascertaining any differences in sequence and/or structure between the target polynucleotide and the reference sequence.

In some embodiments the nucleotides are modified. In some embodiments the modification includes a detectable label. In some embodiments the detectable label is a fluorescent label. In some embodiments the modification is a binding partner to which a detectable label-bearing binding partner binds.

In some embodiments the threshold number of fronts where the extension from origins reach a downstream origin, is close to being all of the fronts, and thus the entire or close to the entire length of the polynucleotide comprises a contiguous read with a negligible number of gaps. This provides long-range genome structure, even through repetitive regions of the genome and also allows individual haplotypes to be resolved. This method can provide highly complete sequences from 1 or just a few cells.

In some embodiments the threshold number of fronts that reach a downstream origin is significantly lower than the number needed for substantially the entire length of the polynucleotide to comprise a contiguous length. Nevertheless, in this case many contiguous reads will be obtained that are longer than a single non-coalesced read, and the gap distance between reads will be visible. These single and coalescent reads, their locations as well as the lengths of gaps between them are then used in computations to assemble a contiguous sequence from a plurality of polynucleotides (copies of the genome, i.e. from multiple cells). Preferably the contiguous sequence is obtained via de novo assembly, using algorithms. However, reference sequences can also be used to facilitate assembly. Some of the algorithms that process information from multiple polynucleotides are used to resolve individual haplotypes covering very long distances. When the threshold fraction is lower, it may not be possible to get a complete genome sequence from a single cell, but a 1 ng amount of genomic DNA (approx. 200 diploid cells-worth) is sufficient. In cases where the threshold fraction is significantly lower, more than 0.5-1 ug of genomic DNA may be needed; for most individual genome sequencing applications it is usually not a problem to obtain such amounts.

In some embodiments, where the genomic DNA is obtained from multiple cells, coalescene can be integrated between reads obtained on a plurality of molecules. Each of the multiple molecules partially overlaps with at least another of molecule out of the multiple molecules and they are aligned by matching common sequences. Each of the partially overlapping molecules share at least a part of one sequence (preferably more than one sequence) with the other molecule. Once alignment has been computationally done, the sequences that are unique to each of the molecules are used to fill the gaps, resulting in a more or completely contiguous assembled sequence.

The method can be implemented on multiple individual (non-clonal) polynucleotides in parallel and the multiple polynucleotides are disposed in such a manner that to a large extent they are individually resolvable over their entire (or substantial part) of their length and overlap between individual polynucleotides is minimal or does not occur at all. Where side-by-side overlap does occur this can be detected by the increase fluorescence from the DNA stain or where stain is not used, by the increased frequency of origins. Where end-on-end overlap does occur, in some embodiments, labels marking the ends of polynucleotides can be used to distinguish juxtaposed polynucleotides from true contiguous lengths.

The polynucleotides can be disposed parallel to a planar surface or perpendicular to a surface. In the case they are parallel to a planar surface, their lengths can be imaged across an adjacent series of pixels in a 2-D array detector such as a CMOS or CCD camera. In the case they are perpendicular to the surface, their lengths can be imaged via Light Sheet Microscopy or Scanning Disc Confocal Microscopy or its variants.

In some embodiments the nucleotides are detectable reversible terminators and the incorporation reactions are conducted in a stepwise fashion, such that once one nucleotide (from the set of all four) is incorporated into an individual growing chain a second nucleotide cannot be incorporated, allowing time for the identity and/or location of the incorporated nucleotide to be detected, before termination is reversed and the next detectable reversible terminator nucleotide is added.

In other embodiments the nucleotides do not comprise a terminator and are labeled via the terminal phosphate and the incorporation reactions are conducted in a continuous fashion, such that once a nucleotide is incorporated, the growing chain is instantaneously ready for the next nucleotide to be incorporated, and the identity of incorporated nucleotide is determined during incorporation and not after incorporation.

The invention provides multiple relatively short reads which run simultaneously along a single long molecule which, when they have progressed far enough, coalesce into a single contiguous long read. Hence, compared to PacBio sequencing whose single long reads proceed serially, the present method obtains segments of the single long read in parallel. Were real-time sequencing chemistry similar to PacBio's to be run in the mode of the present invention, the long contiguous read could be obtained much faster. Were SbS (e.g. Illumina) cyclical reversible terminator chemistry to be run in the mode of the present invention, the read length could be extended by linking together adjacent short reads. Paradoxically, the individual Illumina (or other SBS chemistry) reads can be shortened (for example to 30-60 bases); this is with the proviso that start sites as closely spaced as 30-60 bases apart can be resolved. It is more efficient to run fewer cycles because of gains in cost and speed, and the accuracy is improved because phasing is avoided. Several detection methods, such as scanning probe microscopy (including High Speed AFM) and electron microscopy are capable of resolving such distances when the polynucleotide molecule is elongated in the plane of detection. Furthermore super-resolution optical methods such as STED, stochastic optical reconstruction microscopy (STORM), Super-resolution optical fluctuation imaging (SOFI)), Single Molecule Localization Microscopy (SMLM) and “virtual” super-resolution as will be described herein are capable of resolving such distances.

An advantage of the approach over the droplet based partitioning and barcoding approach developed by 10× Inc. is that the genome structure and haplotype information can be obtained by direct visualization of molecules not by inference or by computational reconstruction. A unique advantage of the method is that when conducted efficiently the genome from a single cell can be sequenced and haplotypes therein resolved. Even when the method is not efficient, much fewer copies of the genome are needed for de novo reconstruction of the genome, than needed by approaches that require partitioning and barcoding of molecules. Also, much fewer processing steps are needed as well as less overall reagent use. Furthermore, because the method can work on genomic DNA without amplification, it does not suffer from amplification bias and error and epigenomic marks such as hydroxymethylation are preserved and can be detected orthogonally to the acquisition of sequence.

Another advantage of the present invention is that it enables long reads to be obtained without actually carrying out costly, and time consuming individual long reads. The long reads are obtained by stitching together contiguous short reads instead. A plurality of short reads are simultaneously obtained along the length of a single molecule. In some embodiments the short reads are conducted by taking advantage of the comparatively high accuracy of SbS using reversible terminators, hence the resultant long coalescent reads are of higher accuracy than obtainable by current long read technologies.

Accordingly, in various aspects and embodiments, the invention provides methods of sequencing a single, elongated target polynucleotide molecule. The methods can include the steps of (a) seeding (or initiating) a plurality of separately resolvable origins of polynucleotide synthesis along the single, elongated target polynucleotide molecule; (b) contacting the target polynucleotide molecule with a polymerase and labeled nucleotides; (c) incorporating a labeled nucleotide (e.g., different dye or different oligo sequence), using the polymerase, into a plurality of (e.g., polynucleotide) sequence fragments complementary to the target polynucleotide molecule and originating from the origins of polynucleotide synthesis; (d) identifying and storing the identity and positions of the labeled nucleotide incorporated into each of the plurality of sequence fragments; and (e) repeating steps (optionally b), (c) and (d) until a threshold fraction of adjacent sequence fragments merge and result in continuous sequence reads spanning two or more adjacent sequence fragments. In other embodiments (b) to (d) are repeated, because, the polymerase may be replaced with a fresh one (even if it is a homogeneous reaction, i.e. does not require exchange of reagents) and polymerase and nucleotides if it is not a homogeneous reaction.

In various aspects and embodiments, the methods can be used for phased sequencing where haplotypes are resolved and may include the steps of sequencing a first target polynucleotide spanning a haplotypic branch of a diploid genome using the method of the preceding paragraph; sequencing a second target polynucleotide spanning the haplotypic branch of the diploid genome using the method of the preceding paragraph, wherein the first and second target polynucleotides are from different homologous chromosomes; thereby determining the haplotypes (linked alleles) on the first and second target polynucleotides.

In various embodiments, step (b) comprises simultaneously contacting the target polynucleotide molecule with a polymerase and four types of differently labeled nucleotides.

In various embodiments, step (b) comprises contacting the target polynucleotide molecule with a polymerase and a single type of labeled nucleotide selected from the group consisting of A, C, G, and T/U.

In various embodiments, the single target polynucleotide is a chromosome. In various embodiments, the single target polynucleotide is about 10², 10³, 10⁴, 10⁵, 10⁶, 10⁷, 10⁸ or 10⁹ bases in length. The wheat chromosome 3b is 995 Million bases in length, whilst the largest human is chromosome 1 at 249 million bases. In various embodiments, the single target polynucleotide is single stranded. In various embodiments, the single target polynucleotide is double stranded.

In various embodiments, the method further comprises extracting the single target polynucleotide molecule from a cell, organelle, chromosome, virus, exosome or body material or fluid as a substantially intact target polynucleotide. In various embodiments, the target polynucleotide molecule is elongated/stretched. In various embodiments, the target polynucleotide molecule is immobilized on a surface. In various embodiments, the target polynucleotide molecule is disposed in a gel. In various embodiments, the target polynucleotide molecule is disposed in a micro- and/or nano-fluidic channel. In various embodiments, the target polynucleotide molecule is intact.

In various embodiments the seeding is via a nick. In various embodiments the nick may be sequence-directed (e.g. via a nicking endonuclease) or it may be random (e.g. generated by DNAse1 or induced by combination of light and intercalator dye). In various embodiments the seeding is via a synthetic oligo. In various embodiments the synthetic oligo targets specific sequences. In various embodiments the synthetic oligo is a random primer. In various embodiments the synthetic oligo is a specific sequence primer. In various embodiments promoters for transcription or primer binding sites (PBSs) for template directed DNA synthesis are inserted via transposition.

In various embodiments the origin's 3′ ends from which multiple synthesis reactions proceed can be dispersed over either the sense or antisense strand of an intact or denatured duplex. In various embodiments the direction of synthesis from one origin and another can be in opposite directions depending on which of the strands the origins seed from. In various embodiments determining which of the strands the origin is at is determined after detecting the direction of extension of the chain, after several or several tens or 100s of nucleotide incorporations.

In various embodiments, the merging of adjacent sequence fragments comprises an overlap of at least 5 bases between the adjacent sequence fragments. In various embodiments, the merging of adjacent sequence fragments is determined by the relative positions of the adjacent sequence fragments abutting and/or overlapping. In various embodiments, adjacent the merging of sequence fragments is determined by the sequences of the adjacent sequence fragments overlapping. In various embodiments, the adjacent separately resolvable origins of polynucleotide are separated by about 10, 50, 100, 250, 500, 750, 1,000, 5,000, or 10,000 bases.

In various embodiments, the adjacent separately resolvable origins of polynucleotide comprise natural sequences of the target polynucleotide. In various embodiments, the adjacent separately resolvable origins of polynucleotide comprise synthetic sequences bound to the target polynucleotide. In various embodiments, the method further comprises (f) ascertaining and storing the positions of the first and second locations in a computer memory; (g) storing the position and identity of the differently labeled nucleotides incorporated into the first sequence fragment and the second sequence fragment in step (e); and (h) ascertaining when the first and second sequence fragments coalesce and assembling the stored identity of the differently labeled nucleotides, thereby sequencing the single target polynucleotide.

In various embodiments, the method further comprises computationally trimming an overlapping segment of adjacent sequence fragments. In various embodiments, the method further comprises (f) seeding a second plurality of separately resolvable origins of polynucleotide synthesis along the single, elongated target polynucleotide molecule; (g) contacting the target polynucleotide molecule with the polymerase labeled nucleotides; (h) incorporating the labeled nucleotides, using the polymerase, into a second plurality of sequence fragments complementary to the target polynucleotide molecule and originating from the second plurality of separately resolvable origins of polynucleotide synthesis; (i) identifying and storing the identity and positions of the labeled nucleotides incorporated into each of the second plurality of sequence fragments, thereby determining the sequences and relative positions of the second plurality of sequence fragments; (j) repeating steps (h) and (i) until a second threshold fraction of adjacent sequence fragments merge and result in continuous sequence reads spanning two or more adjacent sequence fragments; and (k) combining the sequence reads from steps (e) and (j), thereby sequencing the target polynucleotide molecule.

Seeding a plurality of separately resolvable origins of polynucleotide synthesis along the single, elongated target polynucleotide molecule and carrying out SbS can be repeated as many times as necessary to obtain the coverage and redundancy of sequencing required.

In various embodiments, the sequence is determined without using another copy of the target polynucleotide molecule or reference sequence for the target polynucleotide molecule.

In various embodiments, the method further comprises computationally trimming an overlapping segment of adjacent sequence fragments. In various embodiments, the method further comprises (f) repeating steps (c) and (d) until a threshold fraction of adjacent sequence fragments overlap and result in redundant sequence reads spanning two or more adjacent sequence fragments. In various embodiments, the method further comprises (g) identifying any inconsistencies in the redundant sequence reads as potential sequencing errors.

In various embodiments, the method further comprises (f) degrading at least a fraction of the plurality of sequence fragments; and (g) repeating steps (c) and (d), thereby resequencing the plurality of sequence fragments. In various embodiments, a 3′ to 5′ exonuclease is used to degrade the fraction of the plurality of sequence fragments. In various embodiments, the differently labeled nucleotides are degradable nucleotides. In various embodiments, the degradable nucleotides are 5′ amide modified nucleotides and are cleaved by acid. In various embodiments, the degradable nucleotides are RNA and are cleaved by RNAses and/or alkali. In various embodiments, the degradable nucleotides are RNA and further comprising the steps of: (f) degrading at least one of the degradable nucleotides to leave an abasic site or nick; and (g) repeating step (c) using the abasic site or nick as an origin of polynucleotide synthesis. In some embodiments the 3′ends are enzymatically repaired before repeating step (c).

In various embodiments, the method further comprises sequencing the genome of a single cell. In various embodiments, the method further comprises releasing the polynucleotides from a single cell into a flow channel. In various embodiments, the walls of the flow channel comprise passivation that prevents polynucleotide sequestration. In various embodiments, the passivation comprises a lipid, polyethylene glycol (PEG), casein and or bovine serum albumin (BSA) coating.

In general, the methods of the invention include:

a) providing a template nucleic acid;

b) conducting a SbS reaction to obtain a first read from the template; and

c) conducting a SbS reaction to obtain a second read from the template.

d) conducting a SbS reaction to obtain a third read from the template and so on.

Multiple reads are conjoined or are separated by a determinable distance and are preferably carried out simultaneously.

In some embodiments the templates from which individual and coalescent reads are obtained are aligned based on segments of overlap, and a longer “in silico” fragment or ultimately the sequence of the entire chromosome is generated.

In some embodiments of the invention the target polynucleotides are contacted with a gel. In some embodiments the contacting occurs, after elongating the target polynucleotide.

In some embodiments sequences are inserted into the polynucleotides, and act as PBSs to and the 3′ ends of the primers can act as origins.

In some embodiments the sequences are inserted via transposase complexes. In some embodiments the transposase complex acts on the DNA after surface immobilization. In some embodiments sequences are inserted into the polynucleotides, which can act as PBSs or promoters. In some embodiments nicks are created in the polynucleotide. In some embodiments the polynucleotides are denatured.

In some embodiments segments of the elongated polynucleotide are amplified. In some embodiments the amplification occurs via transcription from the inserted sequences. In some embodiments the amplification occurs via the polymerase chain reaction (PCR). In some embodiments one or both of the primers for the polymerase chain reaction are not surface immobilized.

In some embodiments where the primers for the polymerase chain reaction are not surface immobilized, the transposase complex for insertion of the sequences are surface immobilized. In some embodiments the surface contains one or two oligos species for clonal amplification. In some embodiments one oligo is attached to the surface and the other oligo is not attached to the surface.

In some embodiments the oligos are designed not to be specific to any given sequence, they may comprise universal nucleotide analogs or they may comprise highly promiscuous sequence (henceforth both cases referred to as promiscuous oligo). Hence a PBS does not need to be introduced into the target polynucleotide. The promiscuous oligo will bind to any sequence to which it is proximal. Hence when the target polynucleotide is immobilized and elongated on a surface containing one or more types of promiscuous oligo, strand synthesis can be seeded on the polynucleotide.

In some embodiments polymerase reagents, which can act without a extrinsically supplied DNA based primer is used, for example and DNA primase activity can generate a primer is itself Such a polymerase is Tth PrimPol polymerase from the primpol RNA and DNA Polymerase family, as described in WO/2014/14039 which is incorporated herein in its entirety. The advantage of TthPrimPol polymerase is that it is thermostable, processive and can tolerate damaged template polynucleotides; this is important for dealing with FFPE samples.

PrimPols combine primase and polymerase activity in a single protein. This circumnavigates the need to anneal primers to a template polynucleotide to synthesize a complementary sequence; PrimPols create their own primer sequence. Unusually, some PrimPols (e.g. TthPrimPol) are able to copy both RNA and DNA and are therefore the ideal enzyme for sequencing both RNA and DNA from the same sample. In some embodiments the PrimPol polymerase is combined with another Polymerase to initiate and carry out the SbS reaction. In some embodiments the target polynucleotide is fully or partially single stranded. Here the DNA primase capability of PrimPol polymerase is utilized to start the reaction and the other polymerase is involved in extending the reaction. The other polymerase may be a 9° North, DNA Polymerase 1, Sequenase, Taq Polymerase or variants thereof.

In some embodiments the incorporation of each labeled nucleotide into the growing chain is not controlled one nucleotide at a time, and multiple nucleotides can be incorporated. In some embodiments the incorporation of each labeled nucleotide into the growing chain is controlled one nucleotide at a time, so that sufficient time is available in between successive nucleotide additions, to determine the identity of the incorporated base. In some embodiments when distinguishable label or binding partner are not present on the four nucleotides each of the four nucleotides are introduced one at a time. In such embodiments the nucleotides may contain no label. In such embodiments the nucleotides can contain a reversible terminator.

In some embodiments sequences that commonly occur in the target polynucleotide are used to initiate sequencing. This can be one or more of several ultra-frequently occurring sequences in the genome. In this case a fingerprint of a genome, rather than the full sequence of the genome can be easily obtained. In some cases the ultra-frequent sequence is the naturally occurring promoter sequence and acts as a promoter for transcription or a primer-binding site for polymerase based extension. In this case, the sequence of genes can be specifically targeted.

In some embodiments the invention increases the density of sequence information that can be obtained by super-resolving closely packed polynucleotides as well as individual sequencing reactions along the polynucleotides.

In one embodiment the method therefore comprises the steps:

Extracting long lengths of genomic DNA and performing no modification or processing of the DNA

Stretching (elongating) the genomic DNA molecules on a surface

Providing a flow cell (either the stretching has occurred in a flow cell or a flow cell is constructed atop the surface) so that solutions can flow over the DNA stretched on the surface

Creating nicks on the DNA using DNAsel (or optionally an appropriate nicking endonuclease or physical nicking mechanism) or denaturing the DNA and annealing primers

Adding a mix of nucleotides, A, C, G, T each labeled with a distinct label and a reversible terminator to the stretched DNA in a solution comprising a polymerase capable of incorporating the correct nucleotide at each site, in a template directed-manner.

Detecting which nucleotide is added at each location, e.g. using laser Total Internal Reflection (TIR) illumination, a focus detection/hold mechanism, a CCD camera an appropriate objective, relay lenses and mirrors.

The stage on which the flow cell is mounted is translated with respect to the CCD camera so that a multiple of other locations so that genomic molecules or parts of molecules rendered at different locations (outside the field of view of the CCD at its first position) can be sequenced.

Cleaving the terminator across the whole of the array of genomic DNA molecules.

Repeating steps 5-8 for the number of cycles needed for one read to coalesce with another read; erring on the side of making longer reads than necessary to ensure all or the majority of locations have coalesced.

Data Processing:

Processing images,

making base calls

tying base calls to spatial locations

determining which base call locations fit a line

using the obtained information to coalesce sequencing reads to provide a super contiguous read

Using the coalesced reads to assemble a genome.

Providing the coalesced read and assembled genome to the user, preferably via a graphical interface on a computer or smartphone type device.

In the case where higher accuracy is needed one or more of the following approaches are added:

The reads are carried through beyond the coalescence point ideally so that each read is read at least twice.

New start points (e.g. Nicks) are created and the process from steps 4-9 is started again.

In the case where genomic DNA can be extracted from multiple cells many copies of the molecule are displayed on the surface; the results from the same homologs are collected and a consensus read is obtained; homologous molecules are separated, to provide a haplotype or parental chromosome specific reads.

In some embodiments the present invention is distinguished from the prior art, by comprising two or more of the following elements: no prior library preparation before polynucleotides are immobilized; alignment of polynucleotides in one orientation; incorporation of reversible terminators; addition of all four reversible terminators at the same time; the four reversible terminators are each labeled with a different fluorophores; the contiguous sequences in the polynucleotide are constructed by stitching together short reads.

In some embodiments the method comprises amplifying genomic segments within their genomic context comprising:
(a) Inserting primer binding sites (PBS) along the length of the genomic DNA
(b) contacting the genomic DNA with primers that bind to said PBSs
(c) incorporating nucleotides using a polymerase, into a plurality of sequence fragments complementary to the target genomic DNA molecule in a template-directed reaction originating from the primers at the PBSs
(d) Denaturing the complementary strands
(e) Repeating b-d

In some embodiments the genomic DNA is stretched or elongated before or after the insertion of primer binding sites. In some embodiments the stretched or elongated DNA is disposed within a gel or hydrogel. In some embodiments the primer binding sites are inserted via a transposon mediated reaction. In some embodiments the primer binding sites are inserted via an RNA-guided reaction optionally using a Cas protein. In some embodiments the primer binding sites are targeted to specific genomic location via an RNA-guided reaction optionally using a Cas protein. In such embodiments the RNA guides bear sequence that is complementary to the targeted genomic location. In some embodiments rather than insertion of primer binding sites, primers are created by nicking the genomic DNA, for example by using nicking endonucleases.

Further to the above embodiment, in some embodiments the invention comprises a method of amplifying and sequencing genomic segments within their genomic context comprising a single, elongated target polynucleotide molecule comprising:
(a) Inserting primer binding sites (PBS) along the length of the genomic DNA
(b) contacting the genomic DNA with primers that bind to said PBSs
(c) incorporating nucleotides using a polymerase, into a plurality of sequence fragments complementary to the target genomic segments in a template-directed reaction originating from the primers at the PBSs
(d) Denaturing the complementary strands
(e) Repeating b-d
(f) Contacting the amplified segments with primers, polymerase and labeled nucleotides (individually or a mix of all four A, C, G T nucleotides each bearing different labels)
(g) incorporating a labeled nucleotide, using the polymerase, into a plurality of sequence fragments complementary to the target polynucleotide molecule in a template-directed reaction originating from the primers at the primer binding sites on the strands of the amplified genomic segments (segment amplicons)
(h) detecting and storing in computer memory the identity and positioons of the labeled nucleotide incorporated into each of the plurality of sequence fragments
(i) repeating steps g-h, optionally replenishing the polymerase and nucleotides

In some embodiments the labeled nucleotides are reversible terminators.

Various aspects, embodiments, and features of the invention are presented and described in further detail below. However, the foregoing and following descriptions are illustrative and explanatory only and are not restrictive of the invention, as claimed.

### BRIEF DESCRIPTION OF THE DRAWINGS

FIG. 1 The schematic illustrates the general principle of sequencing by coalescence.

The horizontal lines represent a polynucleotide, over six cycles of SbS. Cycle 1 starts with multiple Origins distributed along the elongated polynucleotide. The Origins typically comprise a 3′ OH from which chain extension is initiated. Going from cycle 1 to 6 the chains form each of the multiple origins extend in parallel, incorporating one of the four nucleotides at each location depending on the sequence of the template (nucleotides are represented by colored/shaded balls, each color representing a different base). At cycle 5 much of the template has been copied in SbS but one nucleotide gap remains. In cycle 6 the gap is closed and the independent sequencing reads generated from the multiple origins are at a point that, in processing of the data, the short reads can be coalesced to generate one contiguous long read. Only six cycles are shown here for illustration purposes only, the method is typically implemented using 25 or more cycles for sequencing a genome the scale and complexity of the human genome; detection methods with high spatial resolution (e.g. super-resolution are employed when the individual reads are less than approximately, 700-900 bases).

FIG. 2. The schematic illustrates how a contiguous long read is generated in the case where only a fraction of reads are able to coalescence but multiple copies of the polynucleotide are available. The horizontal lines represent copies of the polynucleotide. The colored/shaded blocks represent sequence reads; the different color/shades represent different sequences. The contiguous long-sequence is generated by integrating the coalesced and non-coalesced reads (the more the reads are coalesced the more confidence there is in the genome assembly and fewer copies of the polynucleotide are needed). One polynucleotide copy is aligned to another, by finding where the polynucleotide copies' share reads across one but preferably more locations along the polynucleotide length. The figure shows that once enough polynucleotides are aligned in this way the sequence can be assembled; this is done by running a computer program of an assembly algorithm.

FIG. 3. The schematic illustrates how origins can be created at set distances apart on multiple polynucleotides. The horizontal lines represent elongated polynucleotides which are uni-directionally aligned. The vertical lines (Originators) represent locations along the substrate. The vertical lines, can be a feature of the flow cell, and may comprise lines patterned from gold ink, onto which thiolated oligos are self-assembled. The vertical lines can be pattern of electromagnetic radiation projected onto the elongated and directionally aligned polynucleotides, which for example induce nicking of the polynucleotide by activating a caged or light-activatable reagent. The blue double-headed arrows illustrate that the distance between Originators. The width of the Originators can be varied and determines the precision to which the origins can be created; the width of the Originators can be sub-micron and the distance between Originators can be several microns; the width of the Originators can be a few nanometres and the distance between Originators can be sub-micron.

FIG. 4 The schematics, a-d illustrate four different ways of creating and extending from origins on elongated polynucleotides. a. The schematic represents the annealing of oligo primers to a single stranded polynucleotide (which may be derived from a denatured double-stranded polynucleotide). The 3′ ends of the primers are then extended (dashed arrow) using a polymerase. b. The schematic represents the extension from the 3′ end of a nick using a polymerase that has a 5′ to 3′ exonuclease activity (or combination of a polymerase with a 5′ to 3 exonuclease). The polymerase removes nucleotides that are downstream of the extending chain as it synthesizes a replacement strand commonly (known as Nick Translation); (iii) shows the coalescence of the upstream nick translation with the origin of the downstream nick translation. c. The schematic represents the extensions from the 3′ end of two nicks using a polymerase that has strand displacing activity (e.g. Phi29, Taq DNA Polymerase and variants, see—BioTechniques, 57: 81-87 2014) and shows the coalescence of the upstream strand displacing extension with the origin of the downstream extension(iii). d. The schematic represents the addition via terminal deoxynucleotidyl transferase (TdT) of a homopolymer sequence (e.g. poly A) to the 3′ end of two nicks (ii). TdT does not use a template, and is a means to add a tail comprising an arbitrary sequence to a polynucleotide. In this case the hompolymer tail comprises a PBS, to which in (iii) a primer binds (e.g. oligo dT). The primer is used to synthesize a strand complementary to the target using a DNA polymerase and thereby conducting SbS; the replaced strand is not shown but can be displaced by a polymerase with strand displacement activity or degraded by an enzyme with 5′ to 3′ exonuclease activity. The schematic illustrates the coalescence of an upstream extension with downstream origin of extension.

FIG. 5 The flow diagrams, a-f illustrate six different embodiments of the invention, a. The steps encompassing DNA extraction and sequencing are shown for an embodiment of reversible terminator based SbS that utilizes DNA PAINT based super-resolution imaging. The incorporation, imaging, and cleavage cycles are repeated for the desired number of times, preferably a number that results in coalescence of reads. The imaging step comprises taking multiple frames (e.g. a movie) which records, over a time period, the pixel locations of on-off binding of imager reagents onto docking sites attached to individual nucleotides that have been incorporated at each of the multiple locations on the elongated polynucleotide; a super-resolution image can then be reconstructed using a stochastic optical reconstruction algorithm (e.g. by using or adapting STORM software) or Single Molecule Localization software, e.g. Thunder STORM. b. The steps encompassing DNA extraction and sequencing are shown for an embodiment that carries out reversible terminator based SbS in which the origin is created by denaturing a double-stranded polynucleotide and binding oligo primers to the single strands. The primers can comprise random primers, sequence specific primers and primers that bind to a PBS inserted via a method such as transposon mediated sequence insertion. A reference oligo can also be used, as an internal marker relative to which the locations of other sequences can be determined. This may be a sequence that occurs ultra-frequently in the genome c. The steps from DNA extraction to continuous simultaneous incorporation and imaging is shown for a real-time SbS (not employing terminators) embodiment. d. The steps from DNA extraction through sequencing are shown for an embodiment which elongates, fixes and denatures a double stranded (the denaturation step is omitted for a single stranded polynucleotide such as RNA) and then carries out a form of sequencing by hybridization in which the location of binding of each hybridizing oligo along the length of the polynucleotide is determined; a complete repertoire of oligos of a given length are tested for hybridization to the polynucleotide, through cycles of hybridization, imaging and denaturation, optionally oligo hybridization is multiplexed, so that a group of oligos are hybridized at each cycle; a reference oligo can also be hybridized at each cycle, as an internal marker relative to which the locations of other oligos can be determined. e. The steps from DNA extraction through sequencing are shown for an embodiment in which PBSs are inserted into a polynucleotide, the DNA is elongated (a fixation step, e.g. UV crosslinking is typically employed after elongation, but is not shown here) and denatured before primers are annealed and are used to originate SbS; in some embodiments the segmental amplification step is omitted and sequencing is done directly on the elongated, denatured single polynucleotides by annealing primers to the PBSs and carrying out a sequencing method of the invention. f. The process for in situ sequencing, inside a cell is shown. The cells are fixed, so that the location of the polynucleotide content is freeze-framed, PBSs for amplification, are inserted into the polynucleotides and amplification (e.g. PCR) is done by annealing primers to the PBSs. Other methods of clonal amplification can be performed as appropriate. This is all done while the polynucleotide remains inside the cell. A reversible terminator based SbS is depicted and is one of favoured approaches when sequencing clonally amplified polynucleotides. The elements of one flow diagram can be replaced with elements of another, for example DNA PAINT can be used for all schemes not involving segmental amplification.

FIG. 6 The schematic represents a method for clonal amplification of segments of an elongated polynucleotide after PBSs have been transposed in and the duplex has been denatured. a. Multiple double stranded insertion sequences are depicted. After denaturation, primers are able to bind to the polynucleotide and amplification is conducted as depicted.

FIG. 7 The schematic illustrates the principle of super-resolution imaging using DNA PAINT (Points Accumulation for Imaging in Nanoscale Topography), as applied to a polynucleotide immobilized, fixed and elongated on a surface.

FIG. 8 The Flow Diagram illustrates the data processing algorithm and its relationship with the experimental sequencing process. One step in the sequencing process is the detection of signals which typically involves acquisition of an image, this occurs after a sequencing chemistry step. Image acquisition can be multi-dimensional and can involve acquisition of multiple images, including a different image for different wavelengths and different. After image acquisition the image is processed, which may involve, flattening the illumination field, subtracting background etc, detection of each elongated polynucleotides directly or indirectly, via the incorporated nucleotide signal, the detection of the brightness of the incorporated nucleotide signal, the detection of the identity of the incorporated nucleotide etc. After image processing, the signal intensities and coordinates are extracted and used for base calling. In some cases the image is not processed in the traditional way, where objects are located in the image, rather the pixel signal intensity and their coordinates are coupled. The base calling comprises a sub-routine in which each signal is characterized (e.g. its representation in images from different filter sets, its brightness, lifetime etc) and compared to the signal characteristic expected for the different bases. When the four nucleotides are added individually and a separate image is taken for each base, then the base calling is simply about, for each base addition, which pixels show a signal of the expected magnitude. After base-calling, for each location (comprising single or multiple pixels) a read is generated by piling up the base calls through the serially ordered stack of images representing the cycles. Information obtained vertically through the cycles can be used to adjust the base calls. If the method is implemented on an ensemble of molecules the possibility of phasing (different molecules of the ensemble being out of synch with each other in terms of which cycle they are at) can be accounted for. Next, the spatially preserved read information is used to coalesce reads that are abutting with one another or are overlapping. If the threshold of coalescence is very high then the assembly process is straightforward and individual polynucleotides can be assembled without reference to any or many more polynucleotide copies. If the threshold of coalescence is lower, then the polynucleotide is assembled using an algorithm that takes integrates single and coalesced reads obtained on multiple polynucleotide copies. Once the contiguous read is assembled, optionally it is displayed in a user-friendly graphical format. If the sequencing is of genomes that comprise multiple chromosomes, the graphical format can include the location of the assembled read on chromosome representations, and with annotations of the location of genes etc. In some embodiments, the same process can be applied to data in which the read are not expected to coalesce, but rather a substantial number of spatially located reads are obtained for a substantial number of polynucleotide copies.

FIG. 9 The flow diagram illustrates an embodiment of the invention, from DNA extraction through sequencing that utilizes reversible terminators. The incorporation, imaging, and cleavage cycles are repeated for the desired number of times, preferably a number that results in coalescence of reads.

FIG. 10 The schematic illustrates how a contiguous long read is generated in the case where multiple copies of the polynucleotide are available and the coalescence is of reads obtained on separate chromsomes. The horizontal lines represent copies of the polynucleotide. The colored/shaded blocks represent sequence reads; the different color/shades represent different sequences. The contiguous long-sequence is generated by integrating the reads from different strands where sufficient overlapping reads are obtained to be able to align overlapping polynucleotide fragments. Once enough polynucleotides are aligned in this way the sequence can be assembled; this is done by running a computer program of an assembly algorithm.

### DETAILED DESCRIPTION OF THE INVENTION

The invention is based, in part, on the discovery that single, elongated target polynucleotide molecules can be sequenced from multiple origins of synthesis that coalesce into continuous sequence reads. Accordingly, the invention, in various aspects and embodiments, provides methods of sequencing a single, elongated target polynucleotide molecule. The methods can include the steps of (a) seeding a plurality of separately resolvable origins of polynucleotide synthesis along the single, elongated target polynucleotide molecule; (b) contacting the target polynucleotide molecule with multiple polymerases and labeled nucleotide(s); (c) incorporating labeled nucleotides, using the polymerases, into a plurality of sequence fragments complementary to the target polynucleotide molecule and originating from the origins of polynucleotide synthesis; (d) identifying and storing the identity and position of the labeled nucleotide incorporated into each of the plurality of sequence fragments; and (e) repeating steps (c) and (d) until a threshold fraction of adjacent sequence fragments merge and result in continuous sequence reads spanning two or more adjacent sequence fragments.

In various embodiments, the method further comprises (f) seeding a second plurality of separately resolvable origins of polynucleotide synthesis along the single, elongated target polynucleotide molecule; (g) contacting the target polynucleotide molecule with multiple polymerases and labeled nucleotides; (h) incorporating labeled nucleotides, using the polymerases, into a second plurality of sequence fragments complementary to the target polynucleotide molecule and originating from the second plurality of separately resolvable origins of polynucleotide synthesis; (i) identifying and storing the identity and position of the labeled nucleotide incorporated into each of the second plurality of sequence fragments, thereby determining the sequences and relative positions of the second plurality of sequence fragments; (j) repeating steps (h) and (i) until a second threshold fraction of adjacent sequence fragments merge and result in continuous sequence reads spanning two or more adjacent sequence fragments; and (k) combining the sequence reads from steps (e) and (j), thereby sequencing the target polynucleotide molecule. Optionally the process of creating multiple origins and carrying out SbS can be repeated as many times as necessary to obtain the coverage and redundancy of sequencing required. As multiple reactions are seeded along a polynucleotide, multiple polymerase molecules are used, one for each site. In some embodiments, polymerase acting on one origin can be replaced with another polymerase during the process of obtaining a read.

Furthermore, the invention, in various aspects and embodiments includes: obtaining long lengths of polynucleotide e.g. by preserving substantially native lengths of the polynucleotides during extraction from a biological milieu; disposing the polynucleotide in a linear state such that locations along its length can be traced with little or no ambiguity, ideally the polynucleotide is straightened, stretched or elongated; before or after disposition of the target polynucleotide in a linear state, creating multiple sites (origins) along the polynucleotide length so that each origins has an origin positioned upstream and an origin positioned downstream of it (with the exception of the two sites most proximal to the two ends of the polynucleotide) and which can prime template directed DNA synthesis, e.g. by nicking to create a 3′ end or annealing an oligo containing a 3′ end; extending each of the 3′ ends, as growing chains, in template-directed reactions, with the strand to be sequenced as the template, using a polymerase to incorporate a nucleotide complementary to the nucleotide present in each of the multiple sites in the target strand to be sequenced; detecting the identity of the incorporated nucleotide at each of the multiple growing fronts; incorporating the next nucleotide into each of the multiple growing fronts and detecting the identity of the incorporated nucleotide; repeating incorporation and detection at each of the multiple sites so that the front of synthesis migrates along the target polynucleotide in a 5′ to 3′ direction until a threshold number of fronts reach at least one downstream origin.

In some embodiments the threshold number of fronts reaching a downstream origin is close to being all of the upstream origins and thus the substantially entire length of the polynucleotide comprises a contiguous read, albeit with a diminutive number of gaps in some of the cases.

In some embodiments the threshold number of fronts that reach an origin that is upstream of them is significantly lower than the number needed for covering the entire length of the polynucleotide to comprise a contiguous length. Nevertheless in this case many contiguous reads will be obtained that are longer than a single non-coalesced read, and the gap distance between reads will be available. These single and coalescent reads, their locations as well as the lengths of gaps between them are then used in algorithms using such information from a plurality of molecules to assemble a contiguous sequence.

Advantages

The advantage of the present invention is that it enables long reads to be obtained without actually carrying out costly, and time consuming individual long reads, by stitching together contiguous short reads instead. A plurality of short reads are simultaneously obtained along the length of a single molecule. In some embodiments the short reads are conducted by taking advantage of the high accuracy of SbS using reversible terminators, hence the resultant long coalesced/coalescent reads are of higher accuracy than obtainable by current long read technologies. The sequencing of a polynucleotide takes less time as multiple reads are being obtained concomitantly rather than a single long read being obtained. As only short individual reads need to be obtained the number of sequencing cycles needed is far fewer than conducted in Illumina SbS. Thus substantially less polymerase and nucleotides are used up. Reducing the standard Illumina 250-300 nt read to 25-30 nucleotides or less, requires at least 10× less reagent use, thus being 10× lower cost.

Another major advantage of the invention is that it enables structural variation of all types to be detected, small or large, including balanced copy number variation and inversions, which are challenging for microarray based technologies, the current dominant approach and at a resolution and scale that can't be approached by microarray, cytogenetic or other current sequencing methods.

Moreover, the method allows sequencing through repetitive regions of the genome. For conventional sequencing the problem with reads through such parts of the genome is that firstly, such regions are not well represented in reference genomes and technologies such as Illumina, Ion Torrent, Helicos/SeqLL, and Complete Genomics deal with large genomes by making alignments to a reference, not by de novo assembly. Secondly, when the reads do not span the whole of the repetitive region, it is hard to assemble the region through shorter reads across the region. This is because it can be hard to determine which of multiple alignments that are possible between the repetitive regions on one molecule with the repetitive region on the other molecule are correct. A false alignment can lead to shortening or lengthening of the repeat region in the assembly. In sequencing by coalescence, when there is complete or near complete coverage of a single molecule by multiple reads either taken simultaneously or one set after the other, a coalescent single read (comprised of shorter reads that are merged) can be constructed that spans the whole of the repetitive region, when the polynucleotide itself spans the whole of the repetitive region. The methods of this invention can be applied to polynucleotides that are long enough to span repetitive regions. Polynucleotides between 1 and 10 Mb are enough to span most of the repetitive regions in the genome. The methods of the invention can be applied to complete chromosomal lengths of polynucleotides from a eukaryote genome as shown in Freitag et al. and attempted in (Rasmussen, et al Lab on a Chip, 11:1431-3 (2011) so it is possible to span all or most of the possible repetitive lengths in the genome.

Target Polynucleotides

The term polynucleotide refers to DNA, RNA and variants or mimics thereof, and can be used synonymously with nucleic acid. A single target polynucleotide is one nucleic acid chain. The nucleic acid chain may be double stranded or single stranded. The polymer can comprise the complete length of a natural polynucleotide such as long non-coding (Inc) RNA, mRNA, chromosome, mitochondrial DNA or it is a polynucleotide fragment of at least 200 bases in length, but preferably at least several thousands of nucleotides in length and more preferable, in the case of genomic DNA several 100s of kilobases to several megabases in length.

In various embodiments, the single target polynucleotide is about 10², 10³, 10⁴, 10⁵, 10⁶, 10⁷, or 10⁸ bases. The single target nucleotide is preferably a native polynucleotide. The single target nucleotide can be double stranded, such as genomic DNA. The single target polynucleotide can be single stranded such as mRNA. The single double stranded target polynucleotide can be denatured, such that each of the strands of the duplex is available for binding by an oligo. The single polynucleotide may be damaged and may be repaired. In various embodiments, the single target polynucleotide is the entire DNA length of a chromosome. The entire DNA length of a chromosome can remain inside the cell without extraction. The sequencing can be conducted inside the cell where the chromosomal DNA follows a convoluted path during interphase. The binding of oligos in situ has been demonstrated: B. Beliveau, A et al Nature Communications 6 7147 (2015). Such in situ binding oligos can act primers or origins to seed strand synthesis to carry out SbS from multiple locations.

Polynucleotide Elongation

In various embodiments, the method further comprises extracting the single target polynucleotide molecule from a cell, organelle, chromosome, virus, exosome or body fluid as an intact target polynucleotide. The target polynucleotides often take up native folded states. For example genomic DNA is highly condensed in chromosomes, RNA forms secondary structures. In various embodiments of the invention steps are taken to unfold the polynucleotide. In various embodiments, the target polynucleotide molecule is rendered in a linear state so that its backbone can be traced. In various embodiments, the target polynucleotide molecule is elongated. Such elongation may render it equal to, longer or shorter than its crystallographic length (0.34 nm separation from one base to the next). In some embodiments the polynucleotide is stretched beyond the crystallographic length.

In various embodiments the target polynucleotide is disposed in a gel or matrix. In various embodiments the target polynucleotide is extracted into a gel or matrix. In various embodiments the target polynucleotide is extracted inside a microfluidic flow cell or channel.

The terms elongated, extended, stretched, linearized, straightened can be used interchangeably and generally mean that the multiple origins and sites of synthesis along the polynucleotide are separated by a physical distance more or less correlated with the number of nucleotides they are apart. Some imprecision in the extent to which the physical distance matches the number of bases can be tolerated. In cases where the elongation or stretching is not uniform along the whole of the polynucleotide length, the physical distance is not correlated with the number of bases with the same ratio across the entire length of the polynucleotide. This may occur to a negligible extent and can be effectively ignored or handled by algorithms. Where this occurs to an appreciable extent, other measures are required. For example in some segments of the polynucleotide, the stretching may be 90% of the crystallographic length, while in other regions it may diverge by around 50%. One way to handle it is via the assembly algorithm that puts together the contiguous sequence. At one extreme the algorithm, does not require distance data, only the order of the reads. Another way to handle it is by using an intercalating dye such as JOJO-1 or YOYO-1 to stain the length of the polynucleotide, then when the polynucleotide is less stretched in certain segments, more dye signal will be seen over the segment of the polynucleotide compared to a segment where it is more stretched. The integrated dye signal can be used as part of an equation to calculate distances between origins.

In various embodiments, the target polynucleotide molecule is immobilized on a surface.

In some embodiments the polynucleotide is stretched via molecular combing (Michalet et al, Science 277:1518 (1997); Deen et al, ACS Nano 9:809-816 (2015), In some embodiments the molecular combing is done by translating a front of fluid/liquid over a surface. In some embodiments the molecular combing is done in channels using methods or modified versions of methods described in Petit et al. Nano Letters 3:1141-1146 (2003).

The shape of the air/water interface determines the orientation of the elongated polynucleotides. In some embodiments the polynucleotide is elongated perpendicular to the air water interface. In some embodiments the target polynucleotide is attached to a surface without modification of one or both of its termini. In some embodiments the target polynucleotide is attached to a surface via hydrophobic interactions with the termini. In some embodiments the contacting of the polynucleotide with the surface occurs under stringency conditions where the termini are frayed allowing the hydrophobic single stands to be exposed.

In some embodiments the polynucleotide is stretched via molecular threading (Payne et al, PLoS ONE 8(7): e69058 (2013)). In some embodiments the polynucleotide is tethered at one end and then stretched in fluid flow (Greene et al, Methods in Enzymology, 327:293-315),In some embodiments the polynucleotide is tethered at one end and then stretched by an electric field (Giese et al Nature Biotechnology 26:317-325 (2008)).

In various embodiments, the target polynucleotide molecule is disposed in a gel. In various embodiments, the target polynucleotide molecule is disposed in a micro-fluidic channel. In various embodiments the target polynucleotide is attached to a surface at one end and extended in a flow stream.

In some embodiments the extension is due to electrophoresis. In some embodiments the extension is due nanoconfinement. In some embodiments the extension is due to hydrodynamic drag. In some embodiments the polynucleotide is stretched in a crossflow nanoslit (Marie et al. Proc Natl Acad Sci USA. 110:4893-8 (2013).

In some embodiments, rather than inserting polynucleotide into nanochannels via a micro- or nanofluidic flow cell, polynucleotides are inserted into open-top channels by constructing the channel in such a way that the surface on which the walls of the channel are formed, is electrically biased (e.g. see Asanov A N, Wilson W W, Oldham P B. Anal Chem. 1998 Mar. 15; 70(6):1156-6). A positive bias is applied to the surface, so that the negatively charged polynucleotide is attracted into the nanochannel. The ridges of the channel walls do not comprise a bias and so the polynucleotide is less likely to deposit there and can be made with or coated with a material which has non-fouling characteristics, and may be passivated with Lipid, BSA, Caesin, PEG etc. In some embodiments the polynucleotide which is attracted into the nanochannel is nanoconfined in the channel and is thereby elongated. In some embodiments after nanoconfinement the polynucleotide becomes deposited on the biased surface, or on a coating or matrix atop the surface. The surface may comprise Indium Tin Oxide (ITO).

In some embodiments the polynucleotides are not all well aligned in the same orientation or they are not straight, rather take up a curvilinear path over 2D or 3D space; although the same kind of information can be obtained as with straight, well aligned molecules, the image processing task is harder and in the case of molecules taking up different orientations, there is increased likelihood that they will overlap and lead to errors. This however, is a necessary evil when sequencing is conducted on polynucleotides in situ inside a cell.

In various embodiments, the method further comprises releasing the polynucleotides from a single or multiple chromosome, exosome, nuclei or cell into a flow channel.

In various embodiments, the walls of the flow channel comprise passivation that prevents polynucleotide sequestration. In various embodiments, the passivation comprises casein, PEG, lipid or bovine serum albumin (BSA) coating.

In various embodiments, the target polynucleotide molecule is intact. In various embodiments, the intact polynucleotide, when double stranded can contain nicks.

In some embodiments the origins are created before the polynucleotide is elongated. This can be done for example by creating nicks in the polynucleotides when it is in a random coil configuration. In some embodiments the origins are created after the polynucleotide is elongated. Here the polynucleotide can be stretched on a surface and DNasel is added for a short period (titration of the amounts required to give the lengths desired is ideally conducted first).

The origins can be created by making a nick, gap or recess in the target polynucleotide. This can be done enzymatically, providing a 3′ end that is extendable. Nicks can be made all along the polynucleotide. Nicks can be made at specific sequence motifs distributed across the genome using nicking endonucleases. Nicks can also be made randomly across the genome using a DNAse1 enzyme or other substantially random enzymatic or physical nicking mechanism. A suitable physical nicking mechanism includes the light an intercalating dye induced nicking.

The origins can also be created at promoters along a genomic polynucleotide.

The promoters can be integrated into the genomic DNA via transposase mediated insertion of a PBS sequence. The origins can also be created by binding of oligo primers across the length of the polynucleotide. A single primer sequence can be used after transposase mediated insertion of a PBS at multiple locations along the polynucleotide with a density controlled by enzyme concentration and/or reaction conditions. It can also be done by invasion of a duplex by an oligo facilitated by a protein, such as RecA. This can also be done by using RNA guided cas or cas-like CRISPR systems. When the target is not a duplex, as in the case of RNA, oligos, an oligo can directly anneal to a target RNA sequence. When the target is native genomic DNA it can be made single stranded before the oligonuceloitdes are bound. This can be done by first elongating or stretching the polynucleotide asd then adding a denaturation solution (e.g. 0.1M NaOH) to separate the two strands. The oligos can be modified, so that they can form higher stability duplexes. The oligos bear a free 3′ end form which extension can occur. The oligos may be a library of randomers comprising degenerate or universal base positions. The oligos may target specific ultra-frequent target sites in the genome (Liu et al BMC Genomics 9: 509 2008).

The oligos may comprise a library, made using custom microarray synthesis. The microarray made library can comprise oligos targeting specific sites in the genome such as all exons or panels for a particular diseases such as a cancer panel. The microarray made library can comprise oligos that systematically bind to locations a certain distance apart across the polynucleotide. For example a library comprising one million oligos will bind around every 3000 bases. A library comprising ten million oligos can be designed to bind around every 300 bases and a library comprising 30 million oligos can be designed to bind every 100 bases. The sequence of the oligos can be designed computationally based on a reference genome sequence. If for example the oligos are designed to bind every 1000 bases, but after one or a few rounds of nucleotide incorporation it becomes apparent that the distances diverge, it is an indication that structural variation compared to the reference is occurring. A set of oligos can first be validated by using them to originate sequencing on polynucleotides from the reference itself and oligos that fail to bind to the right locations can be omitted from future libraries. The library can comprise oligoribonucleotides to induce nicking as origins using CRISPR (McCaffrey et al Nucleic Acid Research (2015)).

When sequencing is done from a promoter and involves RNA transcription, then an RNA molecule is created during the synthesis process and the transcription complex proceeds in the direction of the next origin. The origins can be created before the polynucleotide is elongated. This can be the case where the polynucleotide is in solution or in a gel and an enzyme that creates nicks or oligos that bind along the nucleotide are added to the solution. Then when the polynucleotide is elongated the origins are already present. The origins can alternatively be created after the polynucleotide is elongated. This can be done by the action of DNasel on a double-stranded polynucleotide. In addition, in the presence of intercalator dye such as YOYO-1, nicks can be created by a light induced/oxidative process. This can be used to generate an ordered array of nicks along the target polynucleotide. This can be done by translating a spot of laser illumination over periodic locations along the polynucleotide. Alternatively, a diffraction grating or a photo-mask can be used to project a pattern of light along the polynucleotide in order to create ordered nicks. Alternatively, the binding of oligos on single stranded polynucleotide can create the origins. A double stranded polynucleotide stretched on the surface can be denatured and the oligos can be bound to act as origins. Once origins, bearing free extendable 3′ termini have been created, a polymerase can be added in solution and each origin can be occupied with a polymerase, which catalyses the template directed incorporation of a nucleotide. In some embodiments the origins are created in the same reaction mix as the polymerase extension mix.

Orthogonal Epigenomic Mapping

Methylation analysis can be carried out orthogonally to the sequencing. In some embodiments this is done before sequencing (as the polynucleotide synthesis carried out in SbS or ligation do not reproduce the epigenomic marks). Anti-methyl C antibodies or methyl binding proteins (Methyl binding domain (MBD) protein family comprise MeCP2, MBD1, MBD2 and MBD4) or peptides (based on MBD1) can be bound to the polynucleotides, their location detected via labels before they are removed (e.g. by adding high salt buffer, chaotrophic reagents, SDS, protease, urea and/or Heparin). Similar can be done for other polynucleotide modifications such as hydroxymethylation, for which antibodies are commercially available. After the locations of the modifications have been detected and the modification binding reagents are removed the sequencing can commence. Analysis of the modification can be done before or after the creation of origins. In some embodiments the anti-methyl and anti-hydroxymethyl antibodies are added after the target polynucleotide is denatured to be single stranded. The method is highly sensitive and is capable of detecting a single modification on a long polynucleotide.

If the target polynucleotide is amplified via the PCR, in some embodiments the methylation analysis is done prior to the PCR. The super resolution methods of this invention can be applied to methylation analysis to obtain fine scale analysis. For example, the antibody, the methyl binding protein or peptide can be tailed with an oligo docking site for on-off binding of DNA PAINT imager strands.

There are no reference epigenomes, for DNA modifications such as methylations. In order to be useful, the methylation map of an unknown polynucleotide needs to be linked to a sequence based map. Thus the epi-mapping methods of this invention can be correlated to sequence reads in order to provide context to the epi-map. In addition to sequence reads, other kinds of methylation information can also be coupled. This includes, nicking endonuclease based maps, oligo-binding based maps and Denaturation and Denaturation-Renaturation maps. In addition to functional modifications to the genome, the same approach can be applied to other features that map on to the genome, such as sites of DNA damage and protein or ligand binding.

Creating Origins

In some embodiments the origins are created by internally nicking a double stranded polynucleotide. Nicking can be conducted by DNAse1 in an essentially random manner that is titrated to give a Poisson distribution around a particular gap distance. The nicks leave a 3′ end which can be used for extension by a polymerase. Nicking can also be conducted via nicking endonucleases. The sites of cutting depend on the organization of the recognition sites in the genome for each nicking endonuclease enzyme. In the case of the frequent cutter, Nt.CViPII there is a good chance that nicking will occur tens of nucleotides apart. Nicking with such ultra-frequent cutters can be titrated to give a Poisson distribution around a favored gap distance. As Nickases cleave at specific motifs they recognize, it can be argued, that this introduces a bias regarding sequencing start sites. However, there are two reasons that Nt.CViPII is a useful reagent for creation of start sites for the purpose of this invention, first its recognition site is short and is therefore occurs frequently in the genome, secondly it also possesses an exonuclease activity, this ensures that a proportion of start sites shift away from the nick sites in a stochastic manner, so that when base incorporation commences the origins is relatively randomly scattered across the genome. Of course parts of the genome where there are long runs of particular dinucleotides, homopolymers or other low complexity sequences, may still not be represented. Nevertheless, the enzyme can be useful for much of the genome. Nicking can also be conducted by a Cas9/guideRNA or a CPf1/guide RNA reaction. This conducted using random (gRNA (focused around a PAM site) or a focused library of gRNA. The library of gRNA can be transcribed from oligos synthesized on a microarray and removed therefrom. The oligo library can be designed in silico and synthesized by a vendor (e.g. CustomArray Inc). The oligo primers can be designed to make the synthesis start sites at specific intervals.

In some embodiments the origins are created by internally nicking or nicking and creating gaps in the polynucleotides, using T7 Exonuclease for example. In some embodiments, after creating a nick, the 3′ side of the nick is tailed by Terminal Transferase, by the addition of a string of one of the nucleotides, A, C, G, or T. This reaction can be run for just long enough to give a length capable of acting as a PBS. The reaction can be stopped by reagent exchange, temperature control or by including terminators (like ddNTP) in the reaction mix at an appropriate ratio to the nucleotides. Once tails dispersed across the polynucleotide have been created, a complementary primer can be added e.g. a oligo d(T) primer when the tail comprises a homo-adenine string. In some embodiments the primer comprises a library which contains oligo d(T) plus all possible 1 to 4 specific bases at the 3′ end, so that the primer anchors at the nicking site, rather than further down the length of the tail. The addition of a strand displacing polymerase can then extend the primer and make a copy of one of the strands of the double stranded polynucleotides. The polymerase extension is done in a manner that allows sequencing to be performed according to the methods of this invention.

In some embodiments the nick creation and tailing is done after the polynucleotide has been elongated. In some embodiments the nicking is done prior to elongation (e.g. in solution space) but the tailing is done after elongation. In some embodiments the nicking and tailing is done before elongation (e.g. in solution space). In some embodiments where the nicking and tailing is done before elongation, the elongation can be done by flowing the polynucleotide in a directional flow over the top of a lawn of oligos complementary to the tails. In the majority of cases the polynucleotide is elongated and then a plurality of the tails are captured by the surface attached oligos so that the polynucleotide is immobilized; the capture oligos are then able to act as primers to invade or recess the duplex and perform SbS; the tails will act as origins for sequencing by coalescence.

In some embodiments the origins are created at the ends of the polynucleotides, by creating a recess at the end. Recesses are found at the ends of the polynucleotide when the polynucleotide is fragmented due to single stranded breaks. The recesses can also be created by restriction digestion. For the purposes of sequencing these short recesses need to be chewed back or further recessed in a 3′ to 5′ manner to expose sequence, so that the SbS of this invention can re-extend and fill back the recessed strand. In some embodiments, origins are created by binding of synthetic sequences to the target polynucleotide. This can occur by strand invasion of modified oligos into double stranded DNA, and can include a Rec protein (e.g. RecA) mediated invasion. The binding of synthetic sequences can also occur directly on single stranded polynucleotides or after a double stranded polynucleotide has been made fully or partially single stranded by denaturation, using alkali for example or by digesting one of the strands of the duplex using an exonuclease. Oligo priming, can be conducted using random (RNA or DNA) primers or a library of primers. The library of oligo primers, can be synthesized on a microarray and removed therefrom. The oligo library can be designed in silico and synthesized by a vendor (e.g. CustomArray Inc). The oligo primers can be designed to make to the synthesis start sites at specific intervals. The synthetic sequences can initiate extension in 5′ or 3′ direction if a ligation based sequencing method is used. However, in embodiments when polymerase extension is used SbS is conducted in the 5′ to 3′ direction.

In some embodiments the origins are automatically created by the polymerase. This can be done by the native Phi29 complex. It can also be done by a Primase enzyme. PrimPol enzyme, carries both functionalities of creating a primer and synthesizing a template directed strand. One suitable PrimPol is the thermostable, bifunctional replicase TthPrimPol from Thermus thermophilus HB27.

Insertion of Origins of Amplification or Sequencing

In some embodiments the sequences are inserted using CRISPR cas9-guide RNA complexes and in this case the sequencing can be targeted. In some embodiments sequences are inserted into the polynucleotides to produce origins. In some embodiments the sequences are inserted via transposase complexes. Transposases, transposomes and transposome complexes are generally known to those of skill in the art, as exemplified by the disclosure of US 2010/0120098, the content of which is incorporated in its entirety herein by reference. A plurality of the insert sequence may be inserted into a target polynucleotide by transposition in the presence of a transposase. In some embodiments, a preferred transposition system is capable of inserting the transposon end in a random or in substantially random manner.

In some embodiments the sequences that are inserted into the polynucleotides can act as PBSs or promoters. In some embodiments, segments of the elongated polynucleotide are amplified. In some embodiments the amplification occurs via transcription from the inserted sequences. In some embodiments the amplification occurs via the polymerase chain reaction (PCR) with the inserted sequences as PBSs (see below).

In some embodiments the primers for the polymerase chain reaction are surface immobilized.

In some embodiments only one of the pair of primers for the polymerase chain reaction are surface immobilized. In some embodiments the primers for the polymerase chain reaction are not surface immobilized. In some embodiments where the primers for the polymerase chain reaction are not surface immobilized, surface immobilized transposase complex is used for insertion of the sequences.

In some embodiments the primers are such that they cannot be displaced by the extension of origin that starts upstream. The primers can bear modifications that prevent their displacement by strand displacing enzymes or modifications that prevent their displacement by enzymes comprising 5′ to 3′ exonuclease activity.

In some embodiments, before the polynucleotide is elongated transposon (Tn) mediated insertion is used to insert PBSs or promoters into the polynucleotides, at a density controlled by reaction condition. In some embodiments the density of insertion can be an insertion every 300 bases on average (the current read length obtainable by Illumina SbS). This corresponds to ˜100 nm when DNA is stretched to approximately its crystallographic length. In various embodiments a hyperactive Tn5 transposase is used which is able to create very frequent insertions. The Tn mediated sequence insertion can occur while polynucleotide is in cell, while it is in a gel (e.g. agarose bead), while it is in solution, either in a tube, well, droplet or a microfluidic conduit. In some embodiments, only one sequence us inserted, but this one sequence can be inserted in at different orientations. In some embodiments the PBS is palindromic, and two extensions, each on opposite strands can be seeded, each travelling in opposite direction.

After Tn mediated sequence insertion the polynucleotide is elongated. In some embodiments the polynucleotide is elongated and immobilized (e.g. by sticking to a surface or within a gel or a matrix) and then Tn mediated sequence insertion is conducted. In some embodiments the transposase reaction requires filling in of ends. In some embodiments when the polynucleotide is immobilized and elongated the completion of the transposase reaction entails fragmenting the polynucleotide. This is the case with the Tagmentation (Epicenter, USA) protocol for transposase mediated sequence insertion and fragmentation. However, because the polynucleotide is elongated already and it is immobilized, the fragmentation is relatively inconsequential, as the order and location of the polynucleotide fragments in the original non-fragmented polynucleotide is retained. In some embodiments, after elongation, the polynucleotide is denatured (e.g. using alkali) to separate a double helix into two strands. In some embodiments, the Tn-mediated insertion is a promoter sequence and the polynucleotide is double stranded genomic DNA. In some embodiments, the Tn-mediated insertion is a PBS and the polynucleotide is double stranded genomic DNA.

In some embodiments the Tn5 complex is able to fragment the target polynucleotide. Tn5 transposes enzyme remains tightly bound to the target DNA after Tagmentation, physically linking adjacent fragments of the polynucleotide. In one embodiment Tagmentation is done in solution without removal of the transposase complex (SDS, protease etc is needed to dislodge the complex) and hence the genomic DNA is not separated into fragments. The long length of genomic to which the Tn5 complex is decorated, is then stretched on the surface. In some embodiments the transposase is then removed by addition of SDS or protease. In addition to Tn5 transposase or hyperactive Tn5 transposase, it will be appreciated that any transposition system capable of inserting a transposon end into a polynucleotide can be used in the present invention.

As an alternative to the Tn inserting a PBS, a promoter can be inserted instead. If a promoter has been included in the transposed sequence, sequencing by transcription can commence. RNA in vitro transcription is conducted on the genomic DNA (SbS can be done during this transcription, see elsewhere in this document). Insertion of the promoter, allows the flexibility to carry out either RNA transcription or template directed DNA synthesis, as the promoter sequence can also act as a PBS to a complementary primer.

Separately Resolvable Origins of Polynucleotide Synthesis

Each origin is separately resolvable. This means that each individual sequence read can be followed independent of interference from other reads. In some embodiments this means that the signals from each origin is optically resolvable from adjacent reads. In order to be resolved using diffraction limited optical imaging, the origins need to be a certain minimum distance apart, and this is approximately half of the wavelength light that is emitted by fluorescent labels associated with the incorporated nucleotides. For an emission wavelength of 600 nm, the limit of resolution is approximately 300 nm which equates to around a 1000 bases if the DNA is stretched out according to a separation of 0.34 nm per base. However other factors such as the numerical aperture of the objective lens and the pixel size of the camera also play a role, as well as the contrast. Super-resolution optical hardware is now available and can be used to resolve beyond the diffraction limit of light. This includes STED and SIM. In various embodiments, the adjacent separately resolvable origins of polynucleotide are separated by about 10, 50, 100, 250, 500, 750, 1,000, 5,000, or 10,000 bases.

In various embodiments, the adjacent separately resolvable origins of polynucleotide comprise natural sequences of the target polynucleotide. In various embodiments, the adjacent separately resolvable origins of polynucleotide comprise synthetic oligos complementary to loci on the target polynucleotide.

Treating Samples for Locational Preservation of Reads

In some embodiments after the polynucleotide is elongated a gel overlay is applied.

In some embodiments after the polynucleotide is elongated it is cast in a gel. For example when the polynucleotide is attached to a surface at one end and stretched in flow stream or by electrophoretic current, the surrounding medium can become cast into a gel. This can occur by including acrylamide, ammonium persulfate and TEMED in the flowstream which when set becomes polyacrylamide. Alternatively gel that responds to heat can be applied. In some embodiments the end of the polynucleotide can be modified with acrydite which polymerizes with the acrylamide. An electric field can then be applied which elongates the polynucleotide towards the positive electrode, given the negative backbone of native polynucleotides.

In some embodiments the sample is crosslinked to the matrix of its environment; this may be the cellular milieu. For example when the sequencing is conducted in situ in a cell, a copy of the polynucleotide may be corsslinked to the cellular matrix using a heterobifunctional crosslinker. This is need when sequencing is applied directly inside cells using a technique such as FISSEQ (Lee et al. Science) which can be adapted, for application to genomic DNA, for example via transposon insertion into the genomic DNA or nicking of the genomic DNA (see below).

Once this is done if amplification is conducted on the elongated target polynucleotide, the spatial location of origin of the amplicons can be preserved. Also if sequencing is done on the amplicons or if the signal from sequencing done directly on the polynucleotide is diffusible, the gel or matrix will preserve the diffusible signal to the location of its origins.

One case where the signal is diffusible is if pyrosequencing is applied to the elongated polynucleotide. Here the signal is generated from the released pyrophosphate which is acted on by ATP sulfyrase and Luciferase which emits the signal. In some embodiments the Luciferase or Luciferase and ATP sulfyrase are immobilized on the surface or in a matrix so that the origin of the base being detected is preserved. In some embodiments the incorporated nucleotides contain modifications that allow them to attach to the matrix, for example they may contain NH2 groups which can be crosslinked to a matrix.

In Situ Segmental Amplification

In certain embodiments prior to genome analysis the invention comprises amplification of contiguous genomic segments in situ, origin to origin. The extension start sites are created at the origins and are used for template directed synthesis in order to amplify the sequence adjacent to an origin or in between two origins.

In some embodiments the region at each origin is clonally amplified (similar to polonies, clusters (see WO2012/106546), DNA nanoballs, rolonies or any other in vitro nucleic acid colony amplified by a polymerase) and the many amplicons at the location can be sequenced as an ensemble using Illumina or other SbS or ligation method. As well as remaining in the original vicinity, in some embodiments amplicons will be elongated. Because multiple copies of the same molecule can now be sequenced the effect of polymerase incorporation error during sequencing is mitigated (although polymerase error can be introduced during the amplification). As modified nucleotides do not need to be incorporated during amplification, a high fidelity polymerase such as Phusion or Pwe can be used.

In some embodiments of the invention the target polynucleotides are contacted with a gel or matrix. In some embodiments the contacting occurs, after elongating the target polynucleotide.

In some embodiments, when amplification is performed on elongated polynucleotides, the amplification is done via many individual amplifications over consecutive segments of the polynucleotide. It is important to not let the amplicons diffuse too far from their segment, such that they traverse into the region containing amplifications from a different segment; a small amount of diffusion is permissible as long as sequencing of amplicons from one segment to another can be drowned out by the bulk SbS signals. In some embodiments the amplification is done in a gel layer (e.g. polyacrylamide, agarose) or via crosslinking the target polynucleotides to an immobile matrix, e.g. inside a fixed cell as done in FISSEQ (Lee et al Science. 343:1360-32014).

In some embodiments the amplification is done at distinct locations on each polynucleotide, separated by a fixed and specific distance. In some embodiments the specific distance is one that is just greater than the diffraction limit of light of the longest wavelength used in the study.

After elongation and denaturation on the surface the polynucleotide (double stranded or denatured) is covered with a gel layer. Alternatively the polynucleotide is elongated whilst is already in a gel environment.

A polymerase chain reaction mix is then added, which contains primers that are complementary to the PBS that have been inserted via the transposase. The primers bind each of the two denatured strands. In some embodiments the primers contain a modification that causes them to crosslink or be immobilized within the surrounding gel or matrix. These strands are akin to the two strands that are obtained after the denaturation step of PCR, but which in this case are elongated and immobilized. The primers then anneal to the strand, which is akin to the annealing step of PCR. Next the primers extend the chain, which is akin to the extension step of PCR. In this first cycle the endpoint of the extension is only defined by truncation (enzyme falling off or stopping) or by the time allowed for the extension step. This concludes the first cycle of PCR on the elongated molecule. The switch from the extension step to the denaturation step can be done by changing temperature or exchanging the buffer (e.g. introduction of denaturation buffer devoid of polymerase and nucleotides for extension). After the first extension, denaturation is done again and, because of the gel or matrix, the extended products cannot diffuse far from the extension site. Then a primer-annealing step is done, either by exchanging buffer so that primers are brought in or by shifting to an annealing temperature, if the primers are already present. Upon shifting to extension buffer and/or extension temperature, primers can the carry out extension. Just as in PCR, the extension can occur again on the immobilized strands, but also on the new strands generated in the first cycle. When the PCR is conducted with a single primer oligo sequence it bind to the PBS on both strands but the extension travels in opposite directions.

In the second cycle the immobilized strands again act as templates, but now the strands synthesized in the first cycle also act as templates. With further cycles exponential amplification is carried out. With 10 cycles sufficient template DNA is obtained to carry out sequencing using Illumina, SOLID or Complete Genomics (Science (2010) 327 (5961): 78-81) reagents and their respective instruments. The instruments needs simple imaging: low cost optics, low cost CCD or CMOS camera and LED or lamp illumination. This is coupled with a fluid handling such as a syringe pump or pressure-driven flow system.

It should be noted that when the polynucleotide that is amplified is initially single stranded, then a complimentary copy is first made before PCR commences. Also if the single stranded polynucleotide is RNA, a cDNA reaction is first conducted and optionally a second strand synthesis is also conducted.

It should be noted that when the aim is to also conduct epigenomic analysis on the target polynucleotide, the methods of this invention that analyze epigenomic marks, need to be conducted directly on that target polynucleotide and not the amplicons where the epigenomic marks are not reproduced. Nevertheless, as the original target polynucleotide remains immobilized it can remain a target for epigenomic labeling reagents despite the presence of the amplicons.

The methylation analysis can be conducted on the single polynucleotides before amplification and sequencing.

In some embodiments primers are in solution. In some embodiments primers are attached to the surface or in a gel or matrix. In another embodiment one primer is in solution and the other primer is attached to the surface or in a gel or matrix.

In some embodiments PBSs inserted into the polynucleotide are bound by surface or matrix tethered primers and DNA colonies are created in a similar way to Illumina clusters. The polynucleotide can be disposed and elongated within an Illumina flow cell comprising Illumina bridge amplification primer oligos and the inserted sequences are complementary to the bridge amplification primers. In this case the Tagmentation kit from Epicenter/Illumina can be used, which inserts the correct PBS sequences; the Tn5 is not however removed, so that the polynucleotide remains contiguously held together. In some embodiments only one of the bridge primers is attached to the surface, the other is in solution.

Clonal Amplification Without Llibrary Preparation

In certain embodiments in situ segmental amplification can be done without sequence insertion. In some embodiments this is done, in the case of genomic DNA, after denaturation of the polynucleotide. In some embodiments random or universal primers are bound to the individual strands of the denatured DNA and amplification can be carried out via the PCR or multiple displacement amplification.

In some embodiments the amplification is done via the creation of nicks in the genomic DNA and followed by strand displacement synthesis from nicks or primers bound to the location where the nicks cause parts of the duplex to peel away, due to the fraying of nicked strands from the duplex. In some embodiments priming is conducted by a surface immobilized primer. The surface immobilized primer can be a sequence that binds to virtually any other segment of DNA substantially irrespective of its sequence. This can be a highly promiscuous sequence such as an all purine oligo that contains the motif GGA. Alternatively, the oligo can be composed partially or fully of universal base analogues such as Inosine, 3-nitropyrrole or 3 nitroindole. Such oligos are able to bind and prime any sequence they come into contact with, especially in combination with a polymerase or polymerase variant that is capable of tolerating some non-Watson-Crick base pairs.

In some embodiments a tail is created at each nick using terminal transferase and amplification is done by binding primers to the tail. Amplification can be done by a multiple displacement amplification method or by the PCR. In some embodiments the primer is attached to a surface or matrix. In some embodiments a nicked polynucleotide is immobilized and stretched on a surface also comprising a lawn of oligo dT primers. Terminal transferase and dATP is added to create tails via extension of the 3′ side of the nicks. The poly A tail then binds to the oligo dT primers. A polymerase with a 3′5′ exonuclease and/or strand displacing activity is added and an immobilized copy of a segment of the polynucleotide is created. This can then be tailed with Poly A and oligo dT primers on the surface can make a copy. This then allows bridge amplification to be conducted. Alternatively a sequence bearing a PBS can be added to the free end of the extensions by an RNA ligase. Another alternative is to use random primers or primers containing promiscuous and/or universal bases to synthesize a complementary strand to the surface extended strand, and continuing an amplification reaction with one surface attached primer and one solution primer.

Alternatively synthesis can be initiated by a polymerase that does not need an intrinsic primer. The native form of Phi29 is able to do this, as well as a polymerases that requires no primer whatsoever, such as TthPrimPol polymerase.

In some embodiments a PrimPol polymerase is combined with Phi29 to conduct, efficient clonal amplification. Here the DNA primase capability of the PrimPol polymerase is utilized to start the reaction and the processive strand displacement activity of Phi29 is used to extend the reaction. In some embodiments the PrimPol polymerase is combined with Phi29 to conduct, efficient clonal amplification. Here the DNA primase capability of PrimPol polymerase is utilized to start the reaction and the processive strand displacement activity of Phi29 is used to extend the reaction.

DNA primase do manifest a preferred sequence context from which to initiate, but the context is just a short tract such as GTCC, which would be expected to occur every few hundred base pairs in non-repetitive parts of the genome and regions that have a relatively even pyrimidine/purine content. rtAPrimPol has only a requirement of NTC (where N is A, C, G or T), which would be expected to occur every 16 bases, frequent enough in most parts of the genome to allow priming from any location.

All methods of next generation sequencing require some processing of sample polynucleotides before they can be sequenced. For example, sequencing by the Oxford Nanopore Technology strand sequencing method requires the attachment of a leader sequence onto the polynucleotide. Most other next generation sequencing methods, such as Illumina sequencing require extensive library preparation steps before clonal amplification can be conducted. These steps include, fragmentation, end polishing and tailing, bead selection, gel selection, adaptor ligation and PCR amplification in solution. An important theme of the methods of the present invention is to eliminate sample preparation. The direct single polynucleotide sequencing methods of this invention, in their simplest form require no processing of the polynucleotide after extraction. Following extraction the polynucleotides are elongated on a surface, a matrix or in fluid, origins of sequencing seeded and sequencing started. Indeed in some embodiments the polynucleotides are not extracted at all, and origin seeding and sequencing occurs in situ inside the cell, which may or may not be fixed. In embodiments where the polynucleotides are amplified, the methods of the invention particularly teach means for streamlining the process, and avoiding library preparation, for example the seeding of in situ amplification directly.

The following publications related to primase/polymerase activity are incorporated herein:

Holmes, A. M.; E. Cheriathundam Et Al.: ‘Initiation Of DNA Synthesis By The Calf Thymus Dna Polymerase-Primase Complex’ J Biol Chem Vol. 260, No. 19, 1985, Pages 10840-6

Lipps, G.; A. O. Weinzierl Et Al.: ‘Structure Of A Bifunctional DNA Primase-Polymerase’ Nat Struct Mol Biol Vol. 11, No. 2, 2004, Pages 157-62; Lipps Georg Et Al: “A Novel Type Of Replicative Enzyme Harbouring Atpase, Primase And DNA Polymerase Activity.”, Embo (European Molecular Biology Organization) Journal, Vol. 22, No. 10, 15 May 2003 (2003-05-15), Pages 2516-25259, Xp002711343, Issn: 0261-4189.

In Situ Targeted Amplification or Targeted Sequencing

In some embodiments one or more specific loci are amplified by using primers for amplification that are specific for the loci of interest.

Therefore, the invention comprises a method for targeted amplification comprising:

Optionally extracting the polynucleotide

Creating sites for template directed polynucleotide synthesis

Elongating a polynucleotide on a surface or in a matrix before or after creating sites for template directed polynucleotide synthesis

Denaturing the polynucleotide

Annealing oligo primers to regions flanking one or more loci, such that each loci can be amplified by PCR

Carrying cycles of PCR

Optionally, if the polynucleotide is disposed on a surface a gel matrix is applied on top

Optionally the amplified loci whose locations are preserved are sequenced, optionally the sequencing is conducted via the methods of the present invention

In some embodiments one or more specific loci are sequenced by using primers for amplification that are specific for the loci of interest.

Therefore, the invention comprises a method for targeted sequencing comprising:

Optionally extracting the polynucleotide

Creating sites for template directed polynucleotide synthesis

Elongating a polynucleotide on a surface or in a matrix

Denaturing the polynucleotide

Annealing an oligo primer to the region upstream of one or more loci, such that each loci can be sequenced

Optionally, if the polynucleotide is disposed on a surface a gel matrix is applied on top

The loci are sequenced

In some embodiments, multiple sequencing origins are created around the locus that is targeted. For example, when the locus comprises a gene or the loci comprise a panel of genes. Sequencing from the origins can be targeted and initiated by a programmable CRISPR mediated reaction. Alternatively, when the target polynucleotide is denatured, targeted sequencing can be initiated by sequence specific oligo primers. The primers can be designed to bind at specific expected distances apart. Then the sequencing can commence until synthesis that has commenced from an upstream origin coalesces with a downstream origin and preferably until it has sequenced through the PBS of the downstream origin (in case variants are present at the primer binding sequence. If there is a structural variant with respect to what is expected from the reference used, then the coalescence will occur earlier or later than expected. If the reaction is run for only a certain number of cycles, a gap may be found between the sequencing fronts from one origin to the next. If a structural translocation has occurred, the insertion sequence will be obtained in the sequence read from an origin that is upstream of the sequence that has been inserted due to the translocation.

Because only a subset of polynucleotides from the complex sample (e.g. whole genome or transcriptome) need to be analyzed when targeted sequencing is done in this way, the polynucleotides can disposed on the surface or matrix at a higher density than usual. So even when there are several polynucleotides elongated within a diffraction limited space, when a signal is detected, there is high probability that it is from only one of the targeted loci. This then allows the imaging required for targeted sequencing to be concomitant with the fraction of the sample that is targeted. For example if the <5% of the genome which comprises exons is targeted, then the density of polynucleotides can be 20× greater and thus the imaging time can be 10× shorter than if the whole genome was to be analyzed.

In some embodiments the parts of the genome that are targeted are specific genetic loci. In other embodiments the parts of the genome that are targeted are a panel of loci, for example genes linked to cancer, or genes within a chromosomal interval identified by a Genome-wide Association study. The targeted loci can also be the dark matter of the genome, heterocrhromatic regions of the genome which are typically repetitive, as well the complex genetic loci that are in the vicinity of the repetitive regions. Such regions included the telomeres, the centromeres, the short arms of the acrocentric chromosomes as well as other low complexity regions of the genome. Traditional sequencing methods cannot address the repetitive parts of the genome, but when the threshold of coalescence is high the methods of this invention can comprehensively address these regions. Even when the threshold of coalescence is low, as the gaps between reads can be determined, and the structure of the repetitive regions can be characterized.

Replica Plating Stretched DNA Segmental Amplicons

Once the elongated polynucleotides have been amplified in situ, they can be replicated by the principle of colony transfer, for example by blotting (as in the Southern Blot) onto filter paper or a nitrocellulose membrane etc. Alternatively, replicates can be made as described in Mitra & Church, Nucl. Acids Res. (1999) 27 (24): e34-e39. The replicates then allow orthogonal processing to be conducted on the polynucleotides. For example, methylation analysis can be conducted on the original but sequencing can be conducted on a replicate. Also, if the replicate is of polynucleotides amplified inside a cell, one replicate may look at DNA whilst another looked at RNA. Also, where the aim is to analyze RNA, but the density of the RNA is very high, one replicate may be used to look at one sub-fraction of the RNA population, and other replicates used to look at other sub-fractions of the RNA population. Such sub-fractions may be generated by using primers anchored from a mRNA poly A tail, e.g. oligo dT-AT etc.

Spatially Ordered Origins

The methods for creating origins along the length of a polynucleotide described in this invention create the origins in a stochastic manner and the result is a Poisson distribution of origins along the length of the polynucleotide. The problem associated with this is, for example that if the imaging resolution is 250 nm, with random creation of origins, even when optimized there will be a spread of distances obtained, some below 300 nm and others above 300 nm. Therefore, in some cases the coalescence will occur with fewer sequencing cycles and in other cases it will require a higher number of cycles. Also, when the separation distance between origins is less than 250 nm apart, the sequencing from the two origins will not be resolved and therefore a mixed read will be obtained (which may require other aspects of this invention to resolve. However an alternative solution is to makes the origins in a manner that is not Poisson limited. This can be done by using a physical mechanism with which it is only possible to create origins at specific locations that are a set distance apart. In one embodiment of the invention the origins are made in a spatially ordered manner as follows:

Transposase complexes are arrayed and immobilized on a surface in a series of parallel lines (e.g. by dip pin nanolithography), which each line having a width of 25 nm and separated by the desired distance (e.g. 300 nm).

The polynucleotide is stretched in an orientation that is perpendicular to the parallel lines

The Transposase complexes intersect with the polynucleotides and make a transposition event with the 30 nm window

The next line intersection makes a transposition within the next 30 nm window

The transposon-mediated sequence insertion then acts as a PBS for direct sequencing or for segmental amplification followed by sequencing.

In a related embodiment, an array of gold nanowires are fabricated and thiol modified universal/promiscuous oligos are self-assembled thereon. The advantage of the universal/promiscuous oligos is that they are able to seed sequencing or amplification at any location along an elongated polynucleotide. The ordered separations along the polynucleotide have substantially no correlation with the organization of sequence along the length of the polynucleotide.

A plurality of polynucleotides can be elongated parallel to an array of lines comprising origin-seeding reagents. The laying of the polynucleotides on the perpendicular lines is essentially random with respect to the sequences along the length of the polynucleotide but what is important is the origins are regularly spaced, give or take a certain number of nanometers, depending on the thickness of the line and the precise location of the oligo that seeds the origin within the width of the line.

Preserving Polynucleotide In Situ Territorial Information

In some embodiments the sequencing methods of this invention are applied in situ inside the cell. This can be done after transposon-mediated insertion of PBSs or promoters. In the case of genomic DNA, the DNA can be nicked. In the case of RNA and genomic DNA after it has been denatured, sequencing can be initiated from random primers. In the case of mRNA, sequencing can be initiated from oligo dT derived primers. In some embodiments the sequencing is done on slices of the cell, obtained for example by a Microtome.

As well as conducting segmental amplification on genomic DNA stretched after extraction from a cell, the amplification process can also be adapted to the genomic DNA that remains inside the cell. In this way Fluorescence in situ sequencing (FISSEQ) can be carried out on the whole of the DNA inside the cell (here the Tn mediated insertion is also carried out inside the cell). Then after amplification, FISSEQ cycles are conducted.

Carrying out the sequencing methods of this invention inside a cell allows one to not only sequence the genomic DNA but also to establish the location of the genomic DNA in the cell. Moreover, when applied to tissues it enables the distribution of somatic variant in the cells of a tissue to be analyzed as well as differences in chromosome organization. This is very important, because different parts of the genome interact with each other inside the cell. For example, enhancers contact genic regions through loops and in situ genome analysis enables such interactions to be seen. Also, the organization of the genome or individual chromosome inside the cell can be visualized or determined. In addition, the process can be conducted on a population of cells grown in a dish (e.g. Fibroblasts or neurons) or on tissue sections. In the case of cells or tissues that are substantially three-dimensional, amplification is done on slices of the cells or tissues.

Sequencing and Incorporation of Nucleotides

The target polynucleotide can have an origin of synthesis, which may be a primer bearing an extendable 3′ end or it may be a nick, gap or recess bearing an extendable 3′ end.

The step of contacting the target polynucleotide molecule with a polymerase and nucleotides can comprise allowing the target polynucleotide to interact with a polymerase and nucleotide in an appropriately buffered solution. The interaction is such that it allows the polymerase to catalyze the incorporation of the correctly matched nucleotide at the 3′ end of the origin. Upon incorporation the sugar ring, base and one phosphate of the nucleotide is added to the growing chain, whilst other phosphates (pyrophosphate from dNTP) of the nucleotide are released.

The polymerase is a polymerase that can carry out template directed synthesis. DNA polymerase enzymes are known for their role in DNA replication, the process of copying a DNA strand, in which a polymerase reads an intact DNA strand as a template and uses it to synthesize a new complementary DNA strand. Reverse Transcriptase enzymes are known for their role in transcribing an RNA polynucleotide into a DNA copy, in which the reverse transcriptase reads an intact RNA strand as a template and uses it to synthesize a new complementary DNA strand. RNA polymerase enzymes are known for their role in RNA transcription, the process of transcribing a DNA strand, in which a polymerase reads an intact DNA strand as a template and uses it to synthesize a new RNA strand. The polymerase conducts the synthesis in a 5′ to 3′ direction. When the nucleotide is modified or labeled the polymerase is of such type that can incorporate the modified nucleotide. The polymerase can be a DNA Polymerase, RNA Polymerase or Reverse Transcriptase. The polymerase can be a polymerase DNA Polymerase 1, Taq DNA Polymerase, Sequenase 2.0, Thermosequenase, 9° North or a mutant thereof (e.g. Therminator) as well as many other polymerases natural or mutant. In some embodiments the polymerase can bear a 5′ to 3′ activity or an exonuclease is provided to produce single stranded template sequence downstream. The polymerase can be BST or Phi 29 polymerase or a variant thereof and the strand displacement of such polymerases can be utilized. Ii some embodiments, the polymerase can extend on the short single strand produced when the 5′ end of a nick is fraying, due to natural base-pair breathing. The polymerase can be any polymerase capable of incorporating the labeled and/or modified nucleotides. In some embodiments the target polynucleotide is rendered sterically free for extension.

The nucleotides can bear a label on the sugar, said label may be attached via a cleavable linker, such cleavable linker may be chemically cleavable or photocleavable. The nucleotide can bear a label on the 2′ or 3′ of the sugar ring, said label may be attached via a cleavable linker, such cleavable linker may be chemically cleavable or photocleavable. The nucleotide may bear a modification or label on both the sugar and the base. The nucleotide may in addition bear a modification on a phosphate. The nucleotides can bear a label on a phosphate, said label may be naturally a leaving group upon incorporation of the nucleotide. The labels on the nucleotide can be fluorescent labels. The labels on the nucleotides can be non-fluorescent partners in a binding pair. The binding pairs may comprise an oligo attached to the nucleotide and a complementary oligo bearing a label. The complementary binding pair bearing a label may be contacted to the nucleotide after the nucleotide has incorporated.

Simultaneous Nucleotide Addition Strategy

In various embodiments, step (b) comprises simultaneously contacting the target polynucleotide molecule with a polymerase and four types of differently labeled nucleotides. Each of the four nucleotides A, C, G, T/U may be deoxyribonucleotides if a DNA strand is being synthesized or ribonucleotides if an RNA strand is being synthesized. Each of the four nucleotides are labeled with a label that can be spectrally resolved or deconvolved from the others or bears a label or modification that can be distinguished from one another by the detection method of choice.

Terminator Reversal Strategy

In the case where controlled stepwise sequencing synthesis is conducted, the nucleotide is modified so that only one nucleotide is incorporated at a time, by using a reversible terminator. The reversible terminator comprises a moiety which inhibits or blocks incorporation of a second nucleotide in the growing chain, until it is removed. In order to chemically block incorporation the terminator is positioned on the 3′ position of the sugar ring. However, a terminator located at the 2′ position of the sugar ring or a terminator on the base can inhibit incorporation of more than one nucleotide. The chemical structure of the linker through which the fluorescent label is attached can be sufficient to inhibit the incorporation of more than one base, and terminators of this type have been developed by Genovoxx, Helicos and Lasergen. Once all the nucleotides added to multiple locations on a polynucleotide and on multiple polynucleotides have been detected, the termination can be reversed. If the termination is due to the linker-fluorescent label structure than only one site needs to be cleaved. But if the label and terminator are on different sites, e.g. the terminator is on the 3′ end and the fluorescent label is on the base, cleavage must act at two sites; Illumina have developed a chemistry in which a single chemical reagent is able to cleave the linkage on both sites and these kinds of nucleotides can be used in the methods of the invention. In some embodiments, the terminator at the 3′end can be removed by a DNA repair enzyme.

Termination Repair Strategy

Typically, the reversible terminator chemistries that are used are not native to DNA structures found in nature and contain modifications that must be removed by chemical or physical cleavage mechanisms, which may cause DNA degradation or DNA lesions. By contrast, in the interests of obtaining long and faithful sequence read-length it is important to retain the DNA molecule in a mild environment throughout SbS and each cycle is highly efficient.

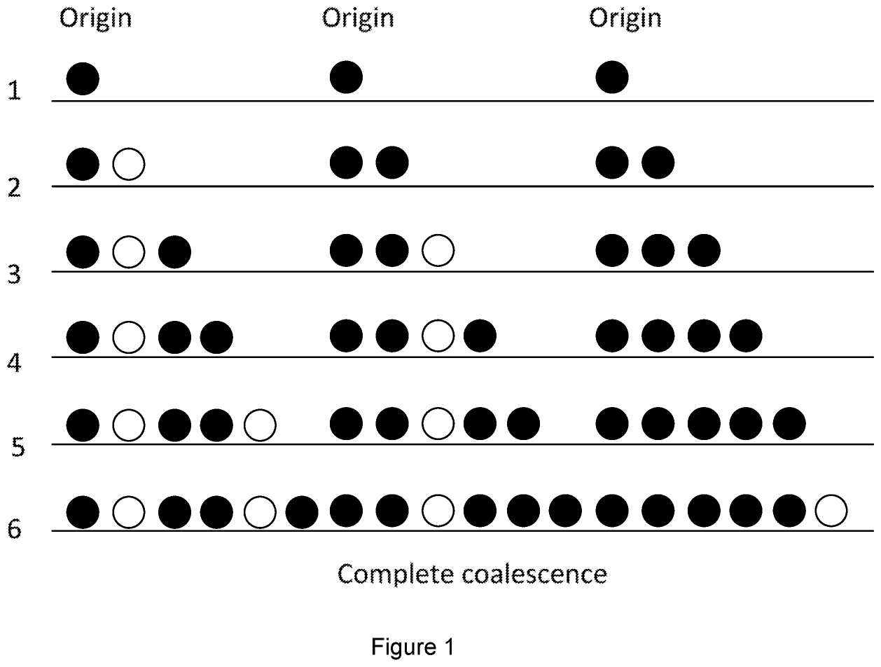

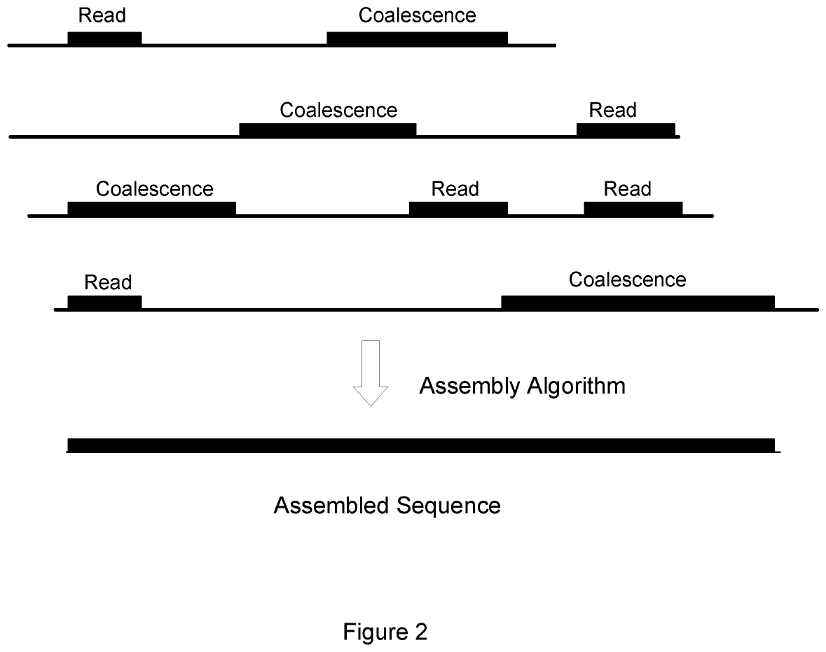

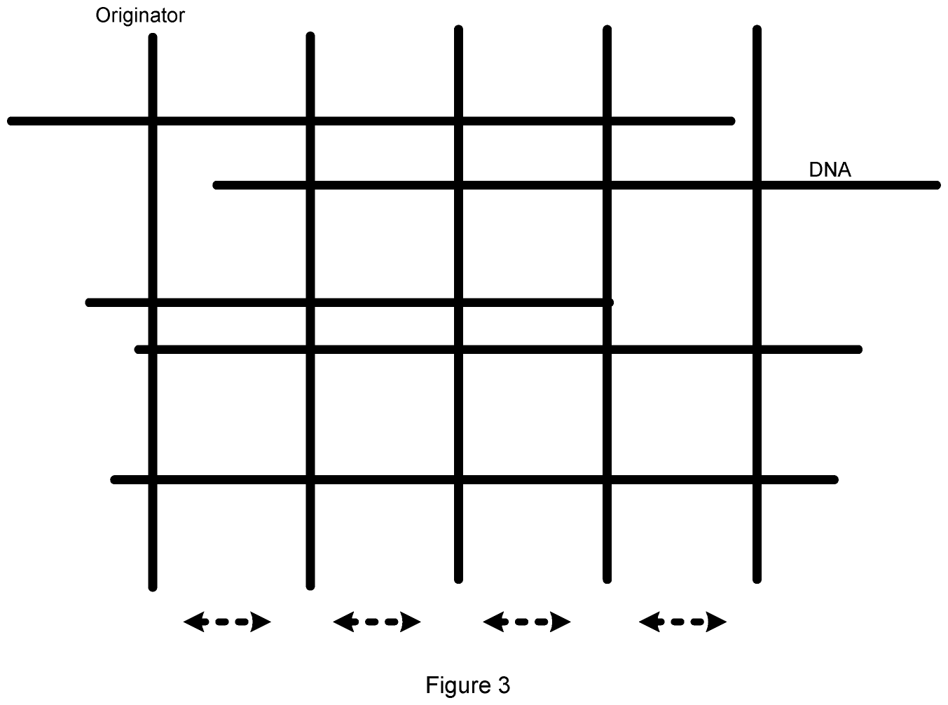

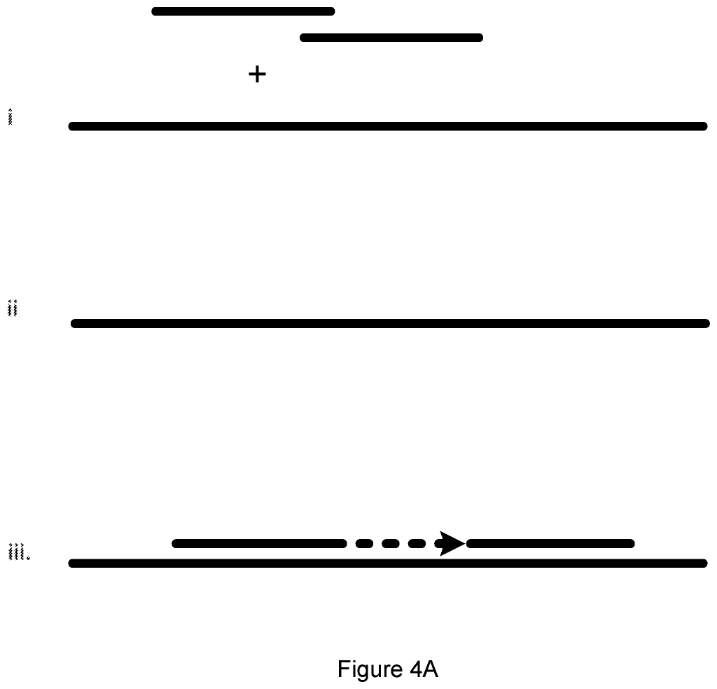

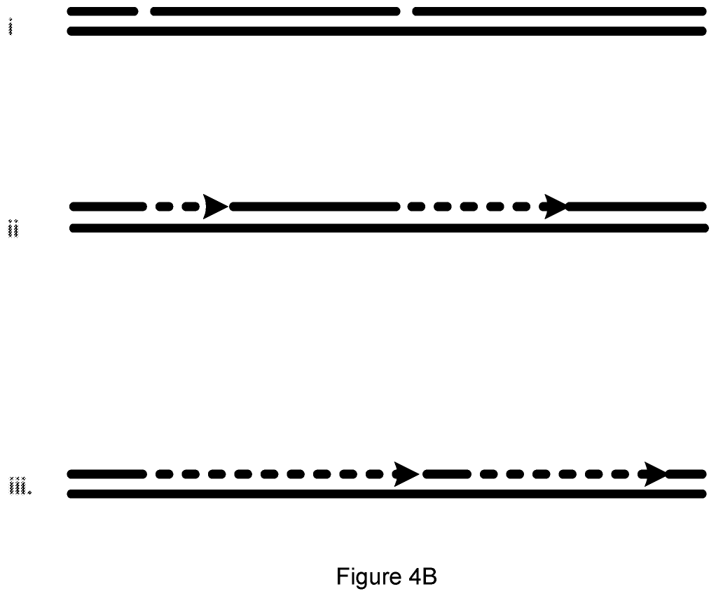

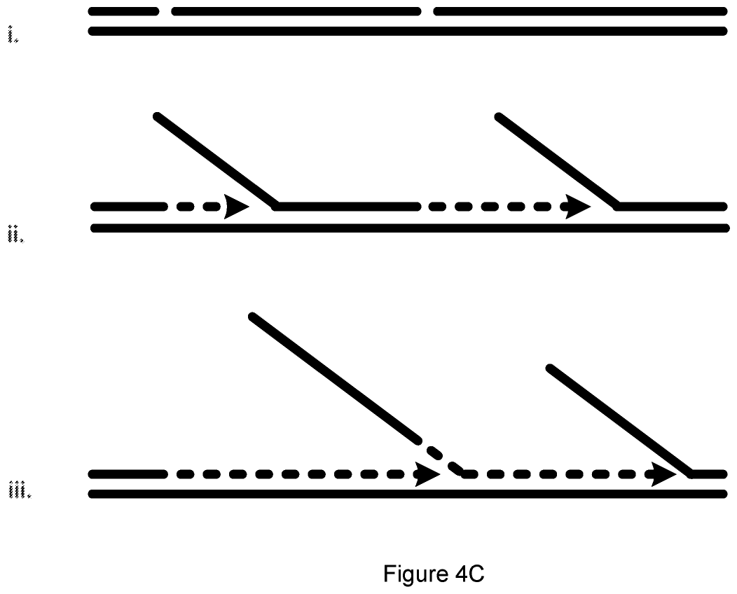

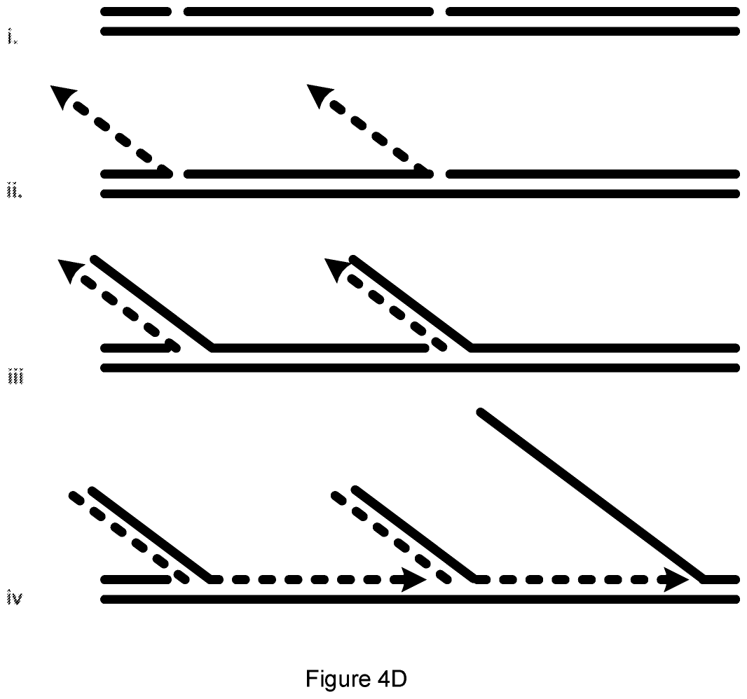

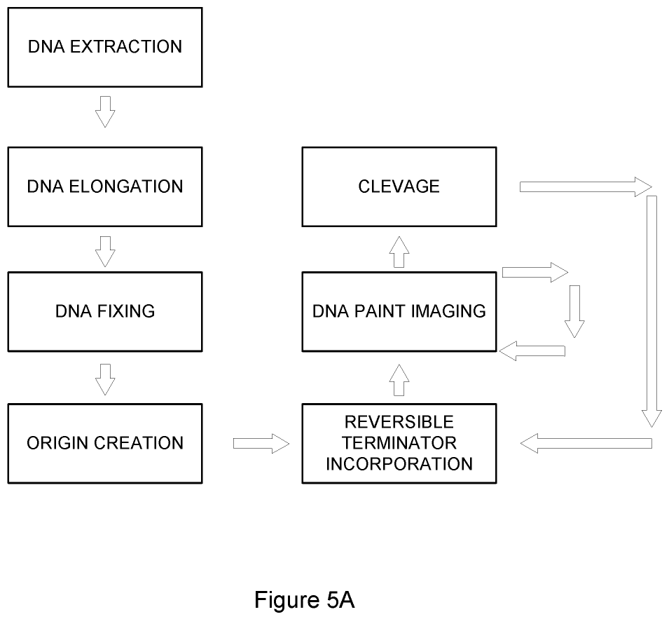

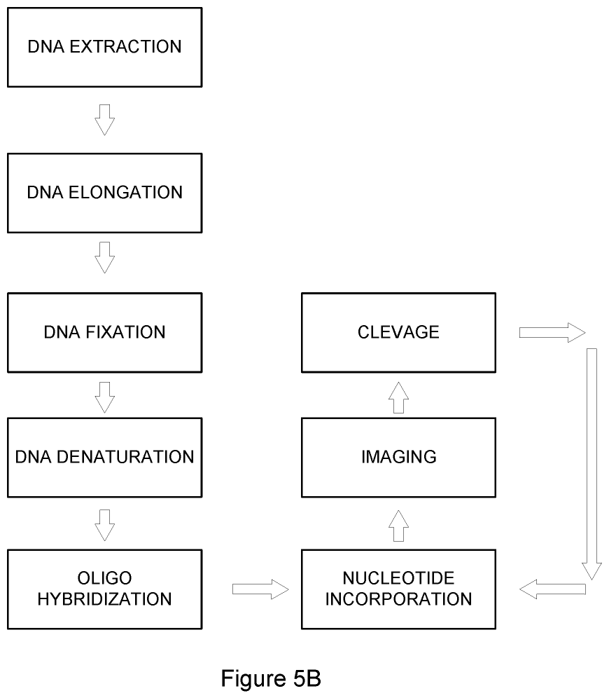

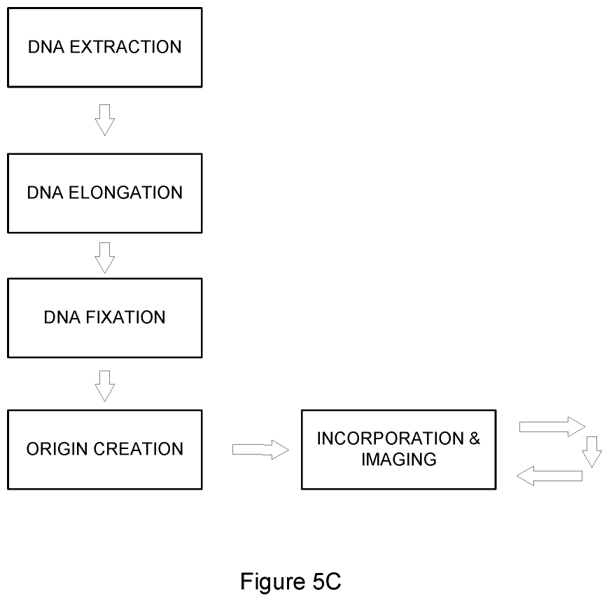

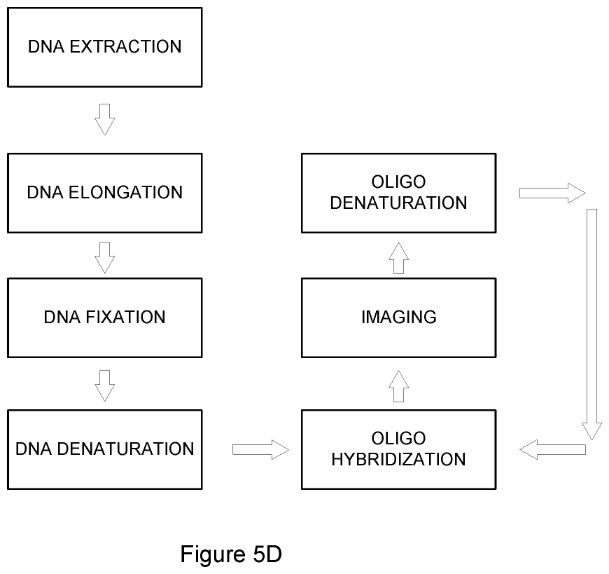

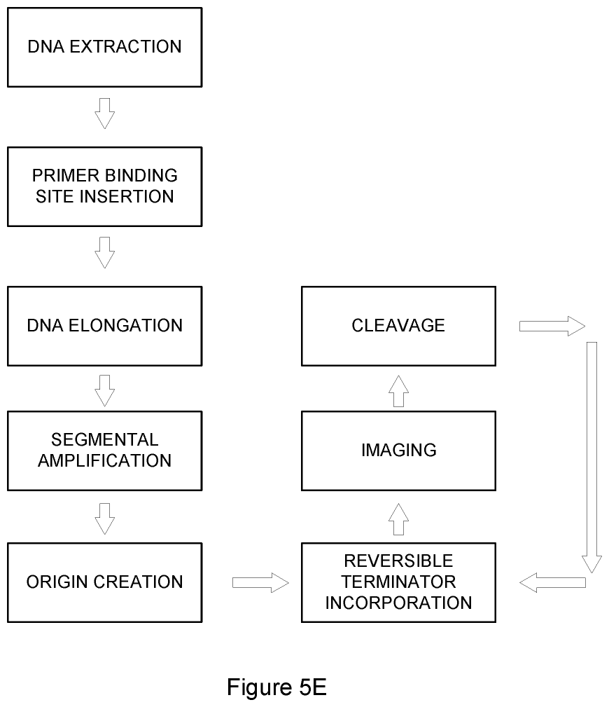

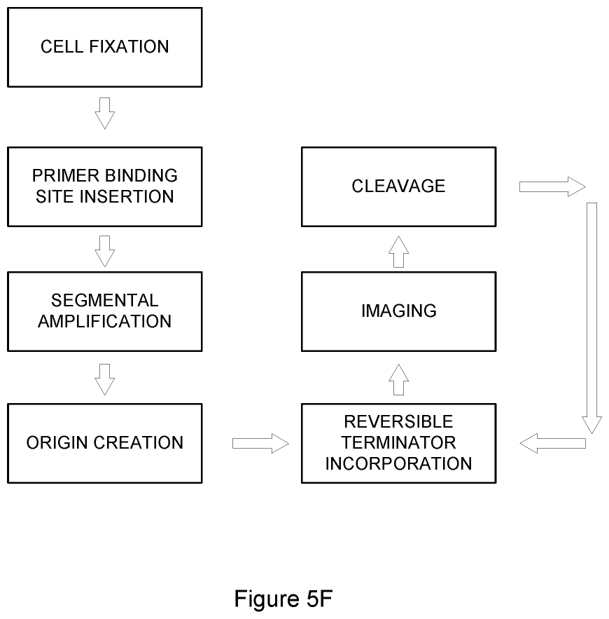

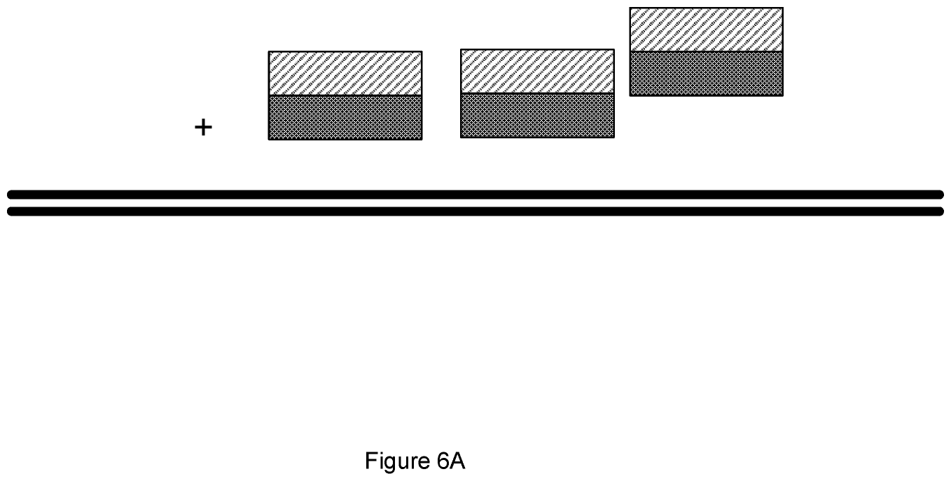

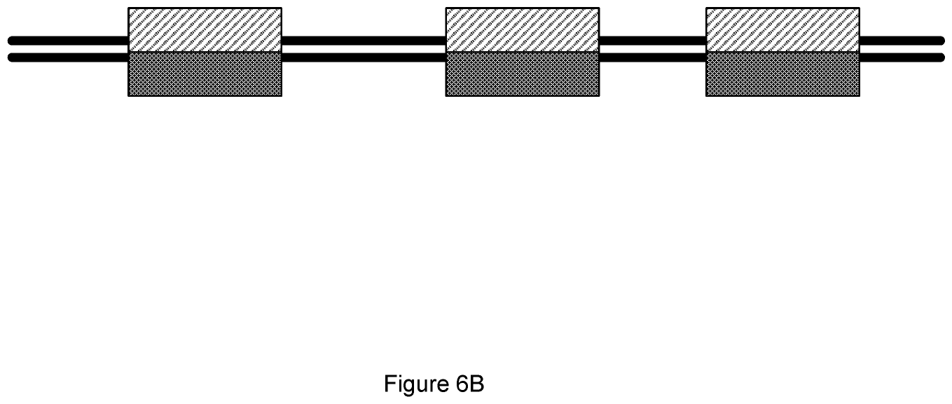

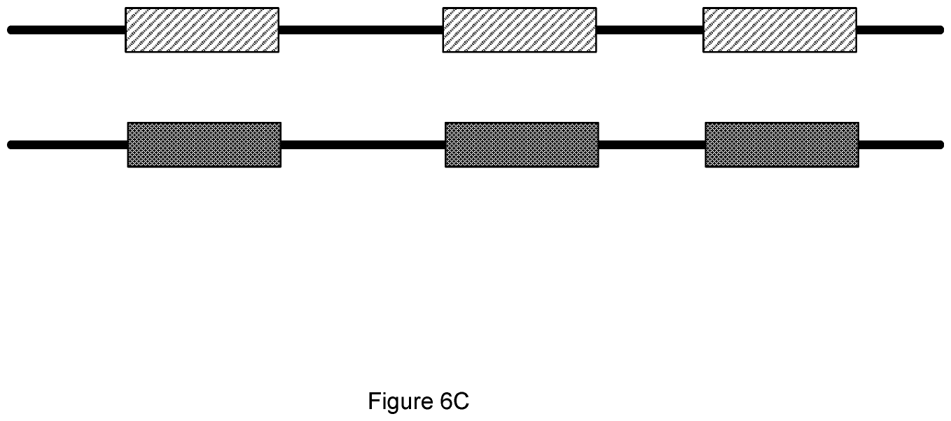

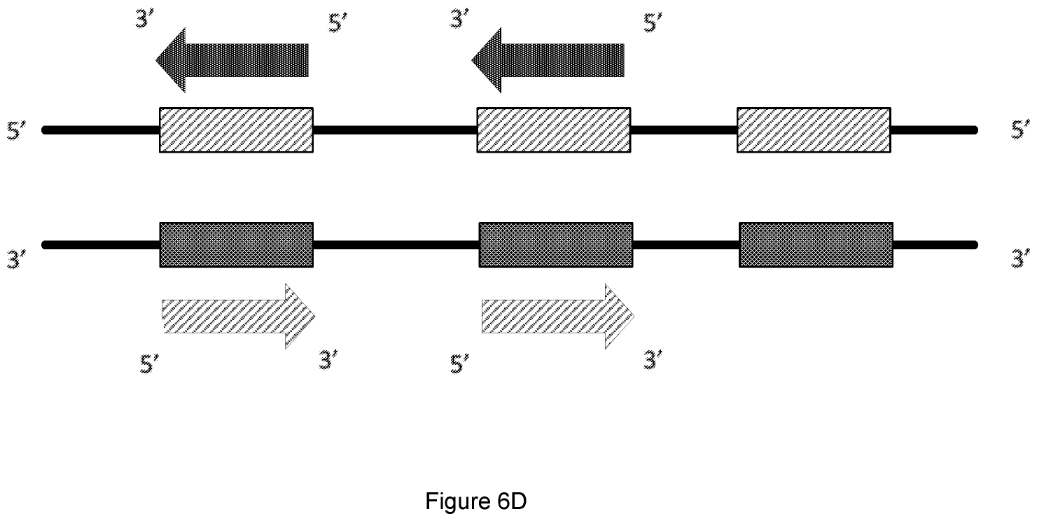

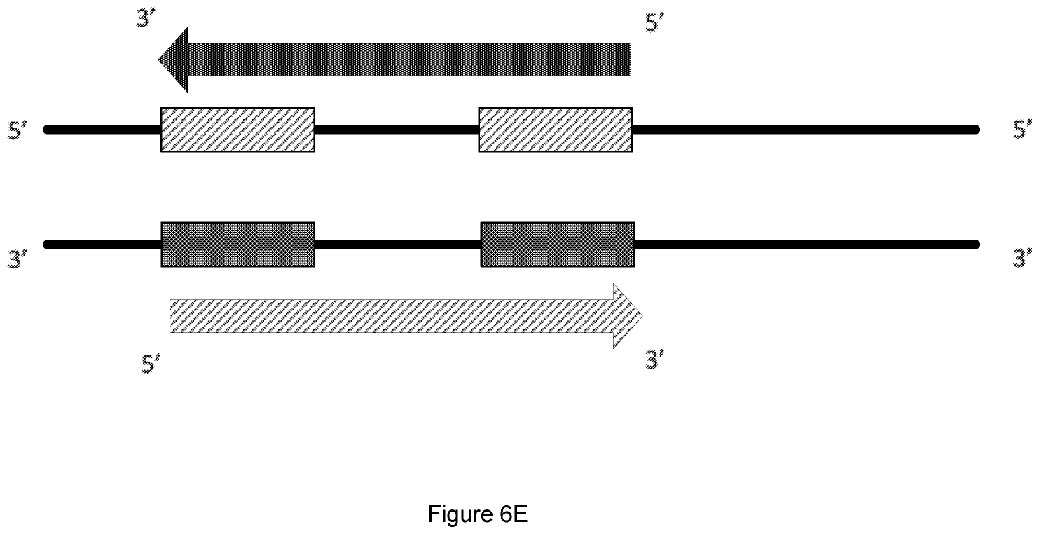

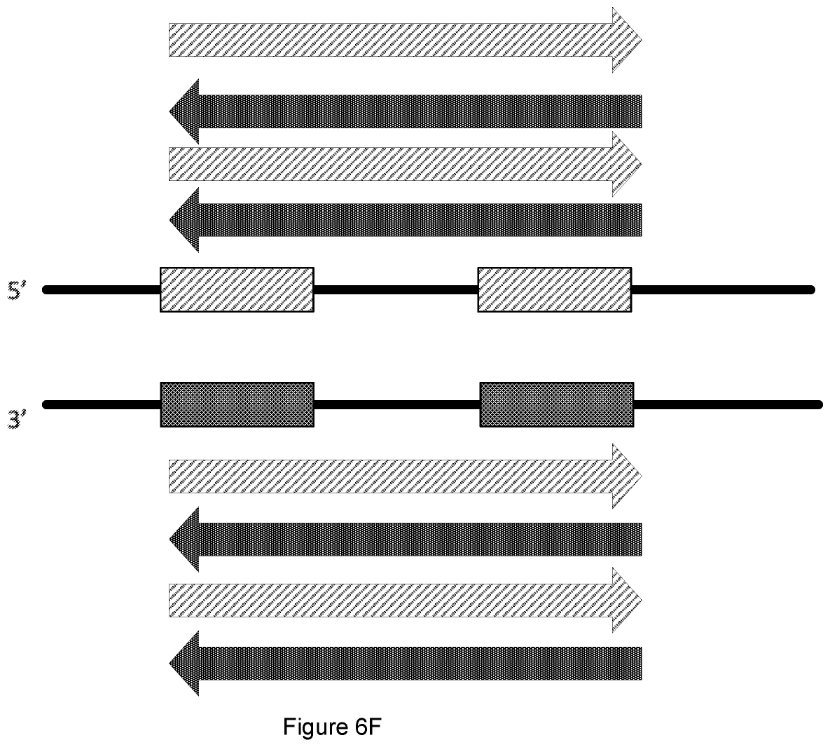

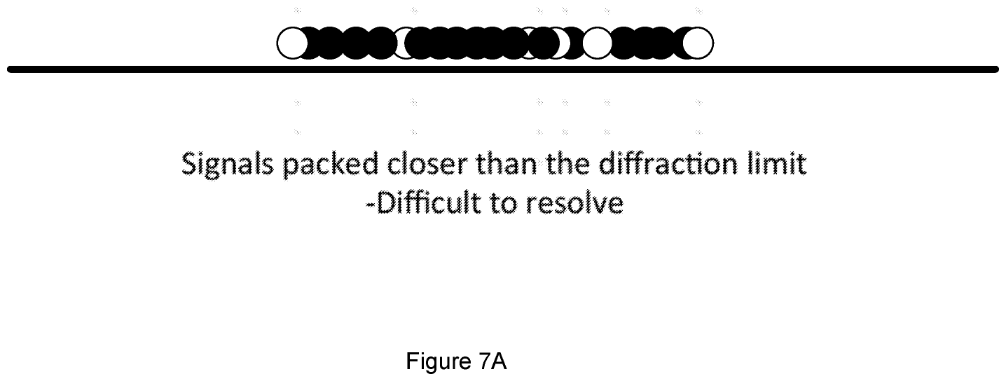

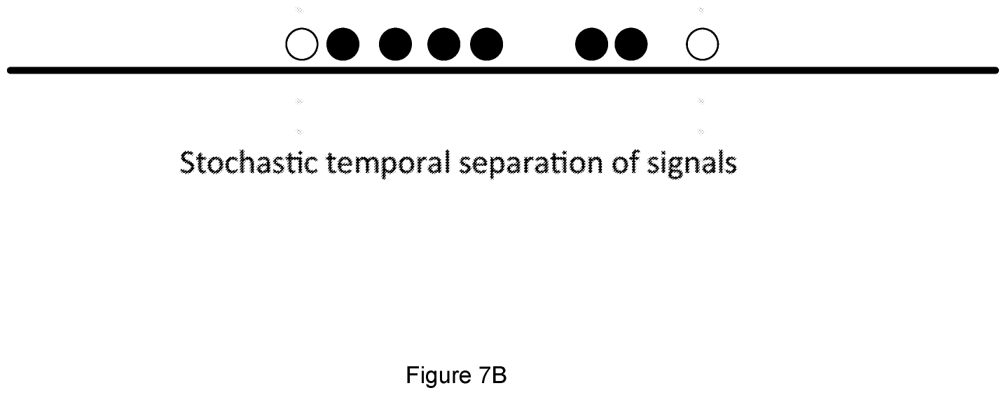

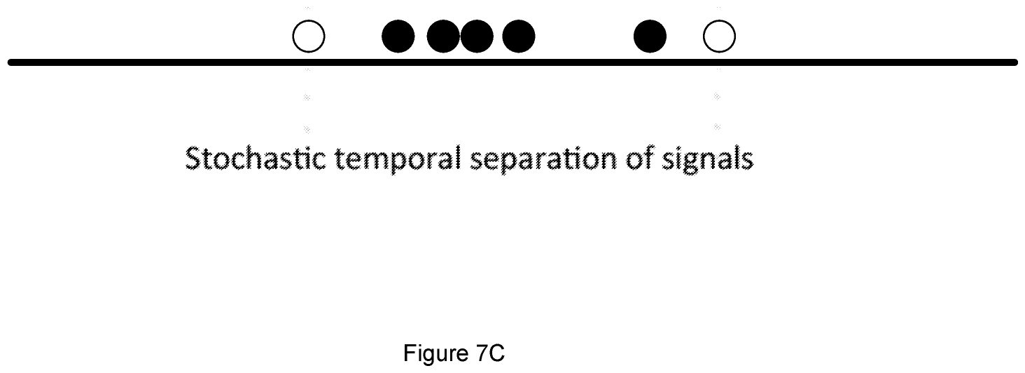

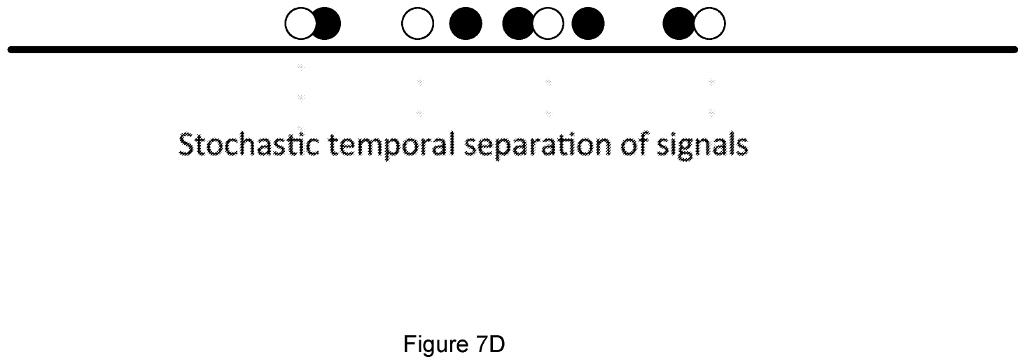

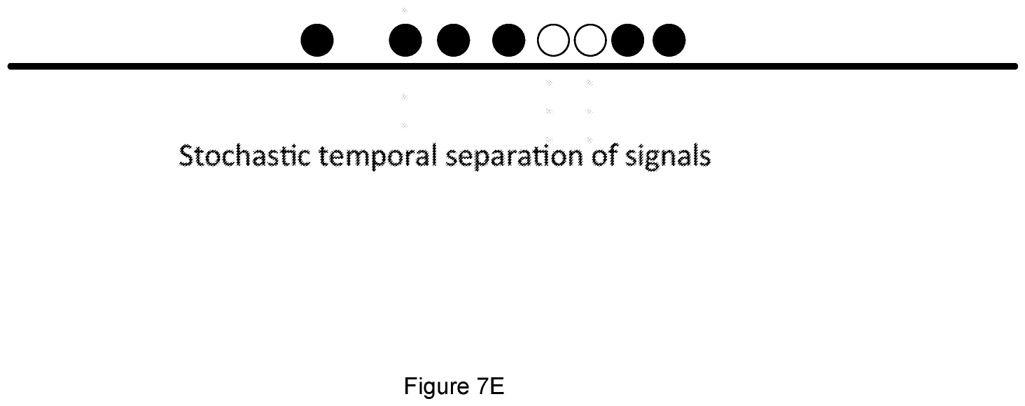

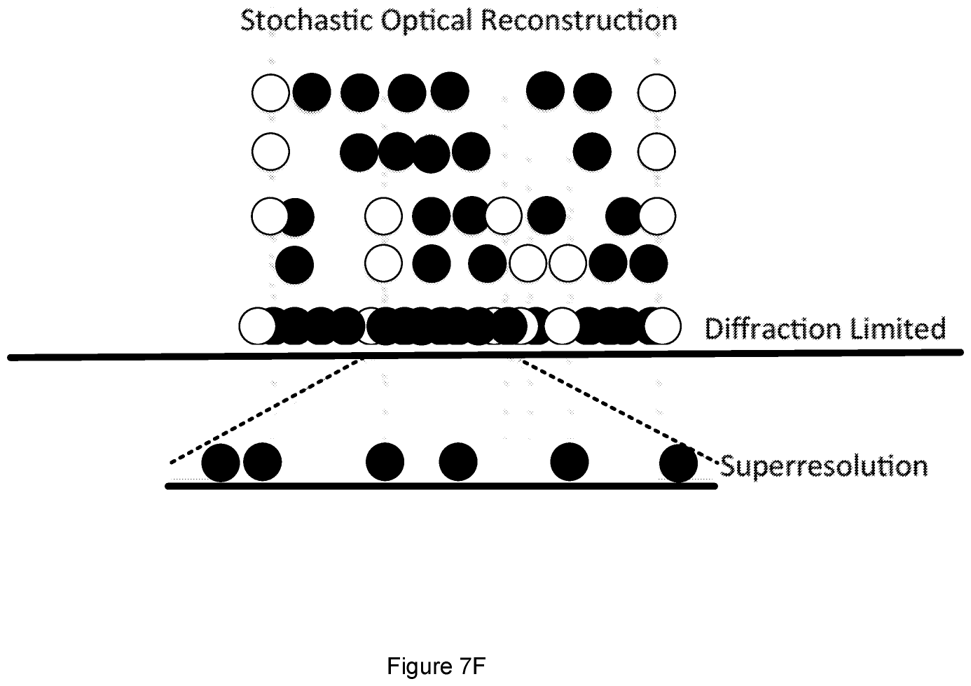

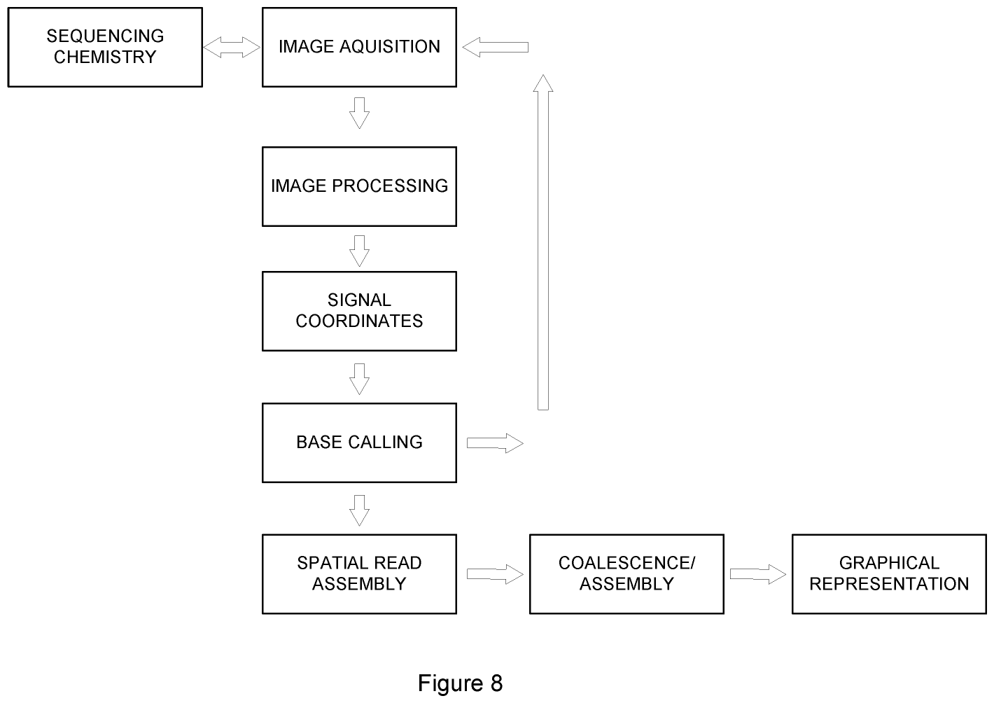

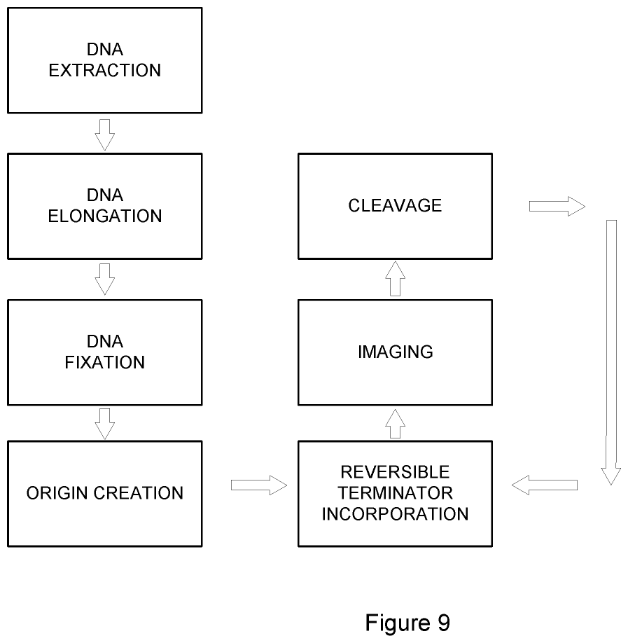

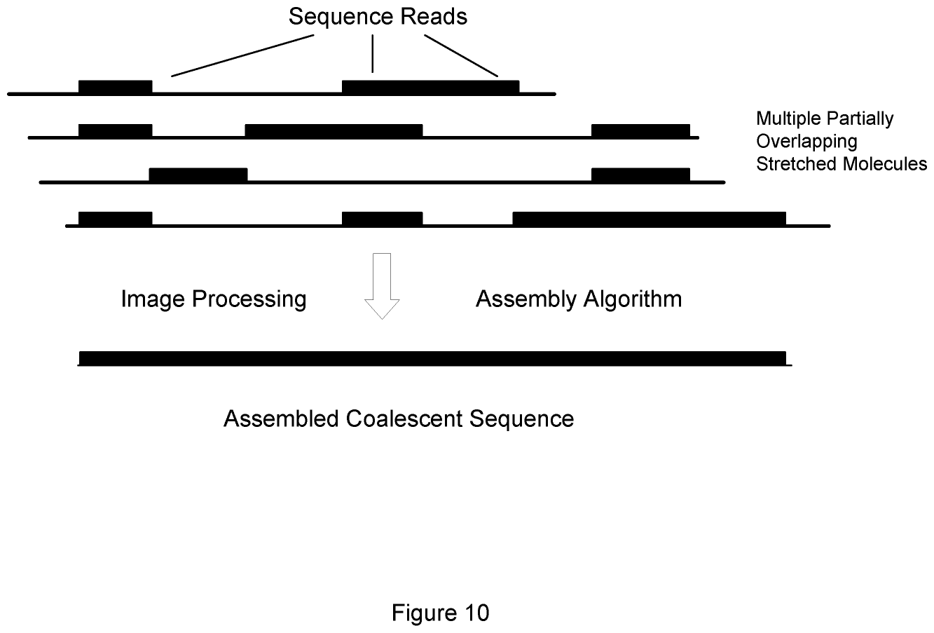

As a critical step towards this important goal, in some embodiments in lieu of a reversible termination strategy, a termination repair strategy is implemented based on the action of enzymes that would normally be involved in maintenance of DNA integrity. In one embodiment this is achieved by using a phosphate at the 3′ position of the sugar ring as a terminator. This mimics a DNA 3′ end after DNA strand breakage, for which nature provides a repair mechanism. The presence of the phosphate group stops the polymerase from adding more than a single nt. Introduction of an enzyme with 3′ phosphatase activity, of which there are many, would result in the repair of the phosphate to a hydroxyl-group allowing synthesis to resume (FIG. 1). For example, Endonuclease IV has a 3′ -diesterease activity and can release phosphoglycoaldehyde, intact deoxyribose 5-phosphate and phosphate from the 3′ end of DNA. Sequence 2.0 and HIV reverse transcriptase can hydrolyze the ester, and amido bonds at the nascent 3′ end of DNA to leave behind the hydroxyl and amine group, respectively. Exonuclease III is known for its ability to remove 3′ blocks from DNA synthesis primers in damaged E. coli and restore normal 3′ hydroxyl termini for subsequent DNA synthesis (Demple B et al, PNAS, 83, 7731-7735, 1986).

Sequencing can be conducted using a two enzyme system. The first enzyme incorporates the 3′ modified nucleotide and the second repairs the nucleotide, making it ready to receive the next nucleotide. The repair enzyme can be added after the polymerase has incorporated the 3′ terminated nucleotide. Alternatively a real time sequencing system can be implemented in which both enzymes are provided simultaneously and after the nucleotide is incorporated, the repair enzyme generates a free OH ready for incorporation of the next nucleotide. However, compared to the real-time sequencing approaches based on terminal phosphate labeled nucleotides, there is a pause between incorporation and repair, which is sufficient to determine which nucleotide has been incorporated. The average time of the pause can be optimized by the reaction conditions and the concentration of the repair enzyme and can be long enough time to carry out detection at one or more locations. Alternatively, the 3′ modification can be cleaved by light, and then if a 3″ OH is not generated it is repaired by the repair enzyme. In some embodiments the 3′ end is not directly labeled with reporter (e.g. fluorophore) but is a binding partner to an imager strand which brings in the label, and in some embodiments DNA PAINT based super-resolution single molecule sequencing is conducted. In some embodiments a homogeneous paused real-time super-resolution sequencing approach is implemented comprising nucleotides with 3′ end binding partner modification, DNA PAINT imager strands, and enzymatic or light cleavable/repairable terminator.

Continuous Incorporation Strategy

In some embodiments, where the incorporation of the nucleotides is not controlled by a terminator, the label may be on the phosphate and no label is present on the sugar or base. The addition of extra phosphates to make a penta- or hexa-phosphate nucleotide and attaching the label to one of the extra phosphates is advantageous and such nucleotides are significantly better incorporated than those to which the label has been attached to a phosphate of a triphosphate nucleotide.

Serial Nucleotide Addition Strategy

In some embodiments the four nucleotides are added serially. In various embodiments, step (b) comprises contacting the target polynucleotide molecule with a polymerase and a single type of labeled nucleotide selected from the group consisting of A, C, G, and T/U. When the target polynucleotide is contacted with a single type of nucleotide, after determination of whether the nucleotide is incorporated or not, it is removed and the next nucleotide can then be added, and so on until all four of the nucleotides have been added. In some embodiments all four of the nucleotides can be labeled with the same fluor. In some embodiments the nucleotide does not contain a terminator. In this case where a homopolymers is present in the target multiple nucleotides are added. Unincorporated nucleotides removed and then the cycle repeated next nucleotide set to be added. Apyrase can be used to degrade of unincorporated nucleotides so that they cannot undergo further incorporation before the next nucleotide is added.

In some embodiments the nucleotide is not labeled and in this case the incorporation of the nucleotide may be via direct detection of the release of pyrophosphate as done in pyrosequencing, it may be via detection of a proton release as done in Ion Torrent sequencing or it may be via detection of a conformation switch in the polymerase. Detection of conformation switch, the fingers opening and closing of the polymerase is the easiest to implement, as the polymerase remains fixed to the elongated target molecule. FRET pairs can be affixed to the polymerase so that a characteristic change in FRET efficacy is seen indicating that a nucleotide has been incorporated (done according to Santoso, Y. et al. Conformational transitions in DNA polymerase I revealed by single-molecule FRET. Proc. Natl Acad. Sci. USA 107, 715-720 (2010). It is also possible to detect differences in the FRET signal depending on which nucleotide is incorporated as described in X. Huang (WO/2010/068884).

Identity and Positions of Incorporated Nucleotides

One aspect of the invention is to store the identity and position of nucleotides incorporated into each of the plurality of sequence fragments. The position of incorporation of a labeled nucleotide along a polynucleotide is determined by a location sensitive aspect of the detector. If a 2-D detector such as CCD is used, the location is determined by the x-y coordinates of the pixels the image is projected on to. If a scanning point detector is used (e.g. in super-resolution STED imaging) then the position of incorporation is determined by the stage coordinates or angle of a galvanometer mirror. A number of computational filters are used to remove spurious binding of labels from what is a true detection event. A label must be correlated with a line that traces through several origins to show the path followed by the polynucleotide; when the path is straight the position that passes the filter falls on the straight line. The detection of a label is only classed as real for the purposes of obtaining sequence reads, when a signal from the location is obtained over multiple sequencing cycles, albeit with tiny shifts in the direction of synthesis. When a 2D image is obtained or is reconstructed, the contour of the polynucleotide is determined in the image and the location of each labeled nucleotide incorporation is determined relative to each of the other labeled nucleotides along the polynucleotide.

The identity of the labeled nucleotide (base calling) is determined in one of two ways depending on how the sequencing is done. If the four nucleotides are differently labeled and used together in one reaction volume, then the identity of the nucleotide is determined by detecting which of the four different labels is detected at the particular location along the polynucleotide. This can be done either by firing four different laser, one for each label, using four different emission filters, one for each label or using a combination of different lasers and emission filters. In this case an image is taken for one wavelength, can be mapped to polynucleotide, then the next and so on. An alternative to serially detecting the four labels is to simultaneously detecting the four labels. This can be done by using a prism to split the emission light to distinct location of a 2-D detector. This can also be done by using dichroic mirrors and emission filters to split the emission wavelengths into four channels, one for each of the four labels. Finally, the emission wavelengths can be split between two and any number of channel, and the intensity of each signal is detected in each channel (signature). In some embodiments a signature spanning the channels for each fluorophore is first obtained and then the signature is used to identify the label and hence the nucleotide from the recorded data.

If the four nucleotides are added one at a time, then the nucleotides can all be labeled with the same fluorophore or not labeled at all and an detection event is used to determine if the nucleotide is incorporated or not, such an event can be of the fingers opening and closing of a polymerase when it incorporates a nucleotide Proc. Natl Acad. Sci. USA 107, 715-720 (2010) or the attachment of a polymerase to the DNA for a period of time indicative of incorporation of a nucleotide (Previte et al Nature Communications 6, Article number: 5936 doi:10.1038/ncomms6936.)

Detection of single fluorescent dyes is susceptible to the idiosyncrasies of each specific dye type. Certain dyes have photophysical characteristics that rule them out as candidate dyes, such as dark states, fast photobleaching, and low quantum yield. Also, the chemical characteristics of the dyes, their structure and whether they carry a charge also affects how well they can be incorporated and the extent to which they non-specifically bind. The choice of dye depends on avoidance of poor photophysical and chemical issues as well as how well they can be excited and detected in a chosen instrument set-up and how well they can be discriminated from the other three dyes. In some embodiments of the invention, other characteristics such as FRET or quenching efficiencies are also important. Fortunately, there are several dye manufacturers and a large list of dyes to choose from. Four dyes that can work well are Atto 488, Cy3b, Atto 655 and Cy7 or Alexa 594. Another four good single molecule dyes that can be used in the invention are shown in Sobhy et al [Rev. Sci. Instrum. 82, 113702 (2011), where a 405 nm, 488 nm, 532 nm and 640 nm laser can be used to excite, Atto 425, Atto 488, Cy3, and Atto647N respectively. Each of the labels indicates a different base identity. Certain dyes need a pulse of light of a different wavelength from their peak excitation wavelength to release them from trapped photophysical states. A number of redox systems are known that minimize the photophysical including: Trolox, Beta-mercaptanol; glucose, glucose oxidase and catalase; protocatechuic acid and protocatechuate-3,4-dioxygenase; methylviologen and ascorbic acid. (see Ha and Tinnefeld, Annu Rev Phys Chem. 2012; 63:595-617). An effective system Fluomaxx is available form vendor, Hypermol (Germany).

In various embodiments, adjacent sequence reads merging comprises an overlap of 1-5 bases between the adjacent sequence fragments. In various embodiments, adjacent sequence reads merging comprises an overlap of at least 5 bases between the adjacent sequence reads.

In various embodiments, adjacent sequence reads merging is determined by the relative positions of the adjacent sequence fragments abutting and/or overlapping. In various embodiments, adjacent sequence fragments merging is determined by the sequences of the adjacent sequence fragments overlapping.

When one of the strands has not been removed to leave one strand of a target duplex, then the situation is complex because sequencing can occur in both directions. This is not a problem when the synthesis reads obtained are not expected to coalescence or the threshold of coalescence is low.

SbS at Multiple Locations Along Elongated Polynucleotide

The invention relates to SbS, which comprises a template-directed chain extension, where a sequencing cycle comprises determination of a single nucleotide in the growing chain. Each sequencing cycle comprises multiple steps and multiple sequencing cycles are conducted to sequence the template (target polynucleotide). In general, sequencing assumes that the target polynucleotide contains nucleotides that are complementary to the ones incorporated (a sequencing error is an example of a case where this assumption would not hold).

The method requires the target polynucleotide to act as a template for the template-directed chain extension, modified nucleotides, which are or can become labeled (e.g. fluorescently) and a polymerization complex. In some embodiments the polymerization complex comprises a polymerizing agent such as a DNA Polymerase, and a 3′hydroxyl terminus. In some a polymerase binds to a nick in one strand of a double stranded polynucleotide and one fluorescently labeled nucleotide analog is added at the nick 3′ end . In some embodiments ternary complexes comprising DNA polymerase, DNA template, and sequencing primer bind at a plurality of sites along the polynucleotide and one fluorescently labeled nucleotide analog is added to the 3′ end of the sequencing primer.

In this case the nucleotides are deoxyribonucleotides. In some embodiments the polymerization complex comprises a polymerizing agent such as a RNA Polymerase and a promoter sequence. In this case the nucleotides are ribonucleotides. In the case of sequencing with an RNA polymerase, the orientation of the promoter determines which strand of the DNA duplex is being sequenced during the course of RNA transcription. Transcription on stretched DNA has previously been demonstrated (Gueroui Z, Place C, Freyssingeas E, Berge B. Proc Natl Acad Sci USA. 2002 Apr 30;99(9):6005-10). In some embodiments the polymerization complex comprises a polymerizing agent such as a DNA ligase and a 3′ hydroxy terminus or a 5′phosphate terminus. In this case the nucleotide is an oligo, optionally with a 5′phosphate depending on the 5′ or 3′ direction of chain extension.

In most embodiments where the polymerization agent is a DNA polymerase, the DNA polymerase lacks 3′ to 5′ exonuclease activity to prevent their being ambiguity about which position along a template is being read at any given incorporation event, because it is not known if the polymerase has chewed back some nucleosides. The exception is embodiments that involve removing incorporated labeled nucleotides and replacing them with an unlabeled nucleotide.

In some embodiments SbS chemistry such as that described in Bentley et al (doi: 10.1038/nature07517) and launched as part of Illumina's initial sequencing chemistry, can be used. Here nucleotides are labeled with a distinct fluorophore on the base with a chemically cleavable linker and there is a terminator on the 3′ of the sugar with a linker cleavable with the same chemistry as the linker attaching the label on the base. An Illumina nucleotide is incorporated at each of the locations along the polynucleotide, their identity and location are detected and then the label and terminator is cleaved allowing the cycle to be repeated. Similarly, the chemistry described by Harris et al (and launched as part of Helicos; initial sequencing chemistry, Harris et al, (Science 320, 106 (2008)) can be used. Typically the incorporation of base labeled nucleotides leaves a chemical scar, a part of the linker , for example that remains on the polynucleotide, and the size and type of the scar can affect the polymerase acting on the polynucleotide and can lead to reduction in the read length that can be obtained. The Lightening Terminator nucleotides developed by Lasergen leave particularly small scars and are therefore effective SbS reagents.

In some embodiments the sequencing reads are obtained thus:

(a) incorporating a plurality of intercalating dye molecules into the target polynucleotide;

(b) contacting the target polynucleotide with a solution comprising a polymerase and four types of differently labeled nucleotides,

wherein each differently labeled nucleotide is fluorescent and can be reversibly terminated and both the fluorescence and termination can be removed by a wavelength of light

(c) incorporating one of the differently labeled nucleotides, using the polymerase, into each location on a chain complementary to the target polynucleotide;

(d) illuminating the target polynucleotide with a first wavelength of electromagnetic radiation, inducing FRET on the intercalating dye and incorporated differently labeled nucleotide partners, and identifying the type of the differently labeled nucleotide incorporated along the polynucleotide via a detection step;

(e) illuminating the target polynucleotide with a second wavelength of electromagnetic radiation, thereby removing the photocleavable label and terminator group; and

(f) repeating steps (a)-(e) as a homogeneous or one pot reaction, thereby sequencing the target polynucleotide.

In some embodiments the sequencing reads are obtained thus:

(a) positioning the target polynucleotide along a focal plane;

(b) contacting the target polynucleotide with a solution comprising (i) polymerase and four types of differently labeled nucleotides,

wherein each differently labeled nucleotide comprises the structure:

N—X-LBP (T)

wherein N is nucleotide, X represents a cleavable linker group chemically bound to LBP and LBP is a Label binding partner and acts as the terminator (T)

or

a separate terminator moiety is provided on the nucleotide also connected to the nucleotide via a cleavable linker

T-X—N—X-LBP

wherein the label comprises the first partner of a binding pair comprising an oligo sequence as a docking site for a DNA PAINT imager and (iii) four distinct DNA PAINT imager strands

(c) using the polymerases to incorporate into multiple chains complementary to the target polynucleotide, one of the differently labeled nucleotides comprising binding partner 1 onto which one of the four binding partner 2 imager strands is able to repetitively bind on and off;

(d) adding the four binding partner 2s

(e) taking a movie under continuous illumination with a first wavelength of electromagnetic radiation, and detecting a persistent signal at specific locations on the polynucleotide, thereby identifying the identity of the differently labeled nucleotide incorporated at those locations;

(e) cleaving the cleavable label/terminator group described in (b); and

(f) repeating steps (b)-(e) thereby obtaining sequence reads along the target polynucleotide.

In some embodiments the DNA PAINT technique is combined with the other aspects described above ore elsewhere in this document. In some embodiments the pronounced or persistent DNA PAINT signal at locations along the target polynucleotide is sufficient to distinguish the signal over background. The DNA PAINT technique provides the background rejection without utilization of BRET, FRET or other proximity based signal enhancement methods, it only requires the persistent signals at locations on the focal plane or surface to be detected. In some embodiments proximity based signal enhancement such as FRET can be combined with DNA PAINT, so that illumination with four separate lasers is not required and so that interference from imager background is reduced.

In some embodiments the sequencing reads are obtained thus:

a) Attaching a FRET/BRET donor (directly or indirectly) to a polymerase;

(b) contacting the target polynucleotide with a solution comprising a polymerase and four types of differently labeled nucleotides,

wherein each differently labeled nucleotide comprises the structure:

L-B—S-T,

wherein S is a sugar, T is a photocleavable terminator group chemically bound to S, and L is a label attached to the base, such label is photocleavable (via a linker so that it can be removed) or is photoinactivatable (e.g., its fluorescence is diminished via photoinactivation or photobleaching) comprising a fluorescence resonance energy transfer (FRET) partner to the FRET donor attached directly or indirectly to the polymerase;

(c) using the polymerases to incorporate the labeled nucleotides into multiple chains complementary to the target polynucleotide;

(d) illuminating (or providing co-factor for BRET) the target polynucleotide with a first wavelength of electromagnetic radiation, inducing FRET/BRET form the label on the polymerase and incorporated differently labeled nucleotide partners, and thereby identifying the type of the differently labeled nucleotide incorporated into each of the locations on the polynucleotide;

(e) illuminating the target polynucleotide with a second wavelength of electromagnetic radiation, thereby removing the photocleavable terminator group and removing the photocleavable label or inactivating the photoinactivatable label; and

(f) repeating steps (a)-(e) as a homogeneous or one pot reaction, thereby obtaining sequencing reads on the target polynucleotide.

In some embodiments the locations of the FRET donor and acceptor are reversed. For example, the donor may be on the nucleotide and acceptor may be on the polymerase or in the duplex.

In some embodiments the sequencing reads are obtained thus:

(a) attaching a Resonance Energy Transfer (RET) donor (directly or indirectly) to a polymerase;

(b) contacting the target polynucleotide with a solution comprising a polymerase and four types of differently labeled nucleotides,

wherein each differently labeled nucleotide comprises the structure:

N-T-Q,

wherein N is a nucleotide, T is a photocleavable terminator group chemically bound to N, and Q is a label comprising a quencher partner to the donor attached directly or indirectly to the polymerase;

(c) incorporating one of the differently labeled nucleotides, using the polymerase, into a chain complementary to the target polynucleotide at multiple locations;

(d) illuminating the target polynucleotide with a first wavelength of electromagnetic radiation, inducing energy/electron transfer between the donor and the incorporated differently labeled nucleotide partners, and thereby identifying the type of the differently labeled nucleotide incorporated;

(e) illuminating the target polynucleotide with a second wavelength of electromagnetic radiation, thereby removing the photocleavable terminator group; and

(f) repeating steps (a)-(e) as a homogeneous or one pot reaction, thereby obtaining sequencing reads at multiple locations on the target polynucleotide.

The quenching mechanism can be a special case for RET, where the energy is not dissipated as light by the acceptor.

The quencher and terminator can both be on the base or both be on the sugar. Alternatively, the quencher can be on the base and the terminator on the sugar.

In some embodiments the sequencing reads are obtained thus:

(a) inserting target polynucleotide into waveguide/plasmonic structure within which the majority of the excitation energy is confined (and/or within which the potential for enhanced excitation exists). See Malicka J, Gryczynski I, Fang J, Kusba J, Lakowicz JR. Increased resonance energy transfer between fluorophores bound to DNA in proximity to metallic silver particles. Anal Biochem. 2003 Apr 15;315(2):160-9.

(b) contacting multiple locations on the target polynucleotide with a solution comprising a polymerase and four types of differently labeled nucleotides,

wherein each differently labeled nucleotide comprises the structure:

N-c-L(T)

or

T-c-N-c-L

wherein N is a nucleotide, c is a cleavable linker, T is a terminator group chemically linked to N, and L is a label chemically linked to N, L(T) is a structure that acts as a label and a terminator wherein L is specific for A, C, G, T/U and c is a cleavable linker

(c) incorporating one of the differently labeled nucleotides, using the polymerase, into chains complementary to the target polynucleotide at multiple locations;

(d) illuminating the target polynucleotide with a first wavelength of electromagnetic radiation, and thereby identifying the type of the differently labeled nucleotide incorporated;

(e) illuminating the target polynucleotide with a second wavelength of electromagnetic radiation, thereby removing the photocleavable terminator group; and

(f) repeating steps (a)-(e) as a homogeneous or one pot reaction, thereby sequencing the target polynucleotide.

United States Patent Application 20180327829, is incorporarted herein by reference.

The above methods of sequencing reads have been described as reversibly terminated stepwise SbS reactions. However, the same mechanisms for background rejection and the ability to carry out a one-pot reaction can also be conducted in a real-time sequencing mode. Here the nucleotides do not bear a terminator and in certain embodiments the label is placed on a terminal phosphate, and the nucleotides may contain additional phosphates beyond the three in natural nucleotides. The polymerase may be Phi29 or a variant thereof and a divalent cation such as Manganese can be used. In such a real-time mode the illumination is continuous and preferably the polynucleotide is rendered in a meandering path (Freitag et al, Biomicrofluidics 9:044114 (2015)) so that multiple locations along a long length can be sequenced within one field of view of a CCD.

In some embodiments reads of 5 bases, each dispersed at multiple locations in the genome is sufficient and unless, the imaging resolution is <2 nm, a low threshold of coalescence will be obtained. Nevertheless, even a 1 or 2 base extension, is sufficient to characterize the structure of a genome. In some embodiments, for example for small, non-repetitive genomes, a read length of 10 bases is more than sufficient to assemble the genome, using de novo assembly algorithms, and 18-25 bases will be sufficient to do the same for a more complex genome, containing repetitive regions, such as the human genome. A read length of 30 bases will require a resolution between origins of ˜10 nm, which is achievable using the Super-resolution methods such as those based on stochastic optical reconstruction, described herein. An origin to origin distance of about 75-90 nt (requiring 75-9 nt read for coalescence) will be amenable to Stimulated Emission Depletion (STED) using for example, Leica TCS SP8 STED 3×, which can have a sub 30nm resolution. This can be implemented using 4 colors or less than four colors. Colors can be resolved in STED by using different laser line combinations, or the same laser lines but fluorophores that can be differentiated based on their lifetime. An origin to origin distance of 250 to 300 bases can be resolved by Structured Illumination Microscopy (SIM). An origin to origin distance of 750-900 nt can be resolved by standard diffraction limited imaging. These resolution requirements are dependent on the emission wavelengths of the fluorophores used, the degree of polynucleotide stretching, the numerical aperture of the objective and the pixel size of the detector/sensor (e.g. CCD).

Imaging and Image Processing

When a fluorescent label has been added to the elongated polynucleotide or to multiple elongated polynucleotides, it can be detected by taking an image with a 2D array detector or using point source detector that is translated with respect to the field of view. The first task is to extract the sequencing data from the images taken at each cycle. Efforts are made to align the stretched molecules along one axis of the 2-D array detector (referred to in this disclosure as a CCD camera, but it can also be a modern scientific CMOS camera) either along the pixel rows or columns of the 2D array detector.

In the case where Time-delayed Integration (TDI) imaging or a line scanner is used, where a continuous image strip is obtained (Hesse J, Sonnleitner M, Sonnleitner A, Freudenthaler G, Jacak J, Hoglinger O, Schindler H, Schutz G J. Single-molecule reader for high-throughput bioanalysis. Anal Chem. 2004 Oct 1;76(19):5960-4.), one embodiment of the invention comprises, matching the direction of the image translation (or stage translation) with the linear direction of elongation of the polynucleotides. This is so that a contiguous image of very long polynucleotides, 100s of microns, several mms or several tens of mms in length can be obtained, and extra computational resources do not need to be devoted to stitching images which can also lead to errors at the image interface.

In some embodiments the system of the invention includes a method for obtaining rapid and accurate long-range images of polymers comprising:

i) Stretching the polymers in one direction

ii) Using a 2-D detector equipped with time-delay integration (TDI)

iii)translating the sample in relation to the detector in the direction of DNA stretching

iv) reading the lines in the direction of translation

wherein the long polymer molecules are analyzed from single long image swathes/strips (without the need for stitching separate frames)

In other cases the ultra-long polynucleotide may be folded into a meandering pattern, through its confinement in a meandering nanochannel (see Frietag et al) and then imaged within the frame of a single CCD or CMOS.

Where the direction of elongation does not correspond to an axis of the 2-D array detector, a first image processing step is done to transform the image so that the lines are aligned along an axis in the image. In some embodiments of the invention, where the polynucleotides are aligned straight in a single orientation, the location of the polynucleotides can be traced by looking at pixels that are activated along a linear axis. Not every pixel needs to be activated, just a sufficient number to be able to trace the polynucleotide over background/non-specific binding to the surface. Signals that do not fall along the axis are ignored. In some embodiments the backbone of the polynucleotide is labeled. For example binding of fluorescent dye such as YOYO-1, Sytox Green, sytox orange, into double stranded DNA, or Sybr Gold into double and single stranded DNA, can be used to trace the polynucleotide.

DNA is typically labeled by a DNA stain/intercalator dye such as YOYO-1.

Instead of a traditional DNA stain, conjugated cationic polymers can be used. Alternatively, no such dye is used but the preponderance or persistence of signal along a linear axis is sufficient to trace out the polynucleotide. The DNA can also be imaged by differential interference contrast (DIC) without DNA stain (Seong et al Electrophoresis, 27:4149 2006).

Super-Resolution and “Super” Single Molecule Localization

There are a number of approaches for resolving optical signals that are closer than the diffraction limit. Firstly, where the characteristic of an emitting object such as quantum dot or a dye are known, it is possible to use the point spread function of the dye to resolve two closely spaced signals along the polynucleotide. This is easier to do when two closely spaced signals are emissions at different wavelength. A number of algorithmic approaches have been described. Secondly, it is possible to resolve the signals by allowing them to photobleach, a stochastic process (J Biomed Opt. 2012 Dec;17(12):126008). Thirdly, there are a number of hardware approaches that have been described and are commercially available, these include scanning optical microscopy, 4Pi, STED, and SIM. In the case of STED, specific compatible sets of fluorophores must be used. A number of molecular approaches have also been described, based on closely spaced signals being temporally separated and this includes STORM (Sub-diffraction-limit imaging by stochastic optical reconstruction microscopy (STORM) M. J. Rust, M. Bates, X. Zhuang Nature Methods 3:793-795 (2006) and specific sets of compatible fluorophores must be used.

Another super-resolution method, DNA PAINT (Jungmann et al Nano Lett. 2010, 10:4756) can also be used in various embodiments of this invention. These approaches can be applied to resolve signals that are normally not resolvable by optical microscopy. In the case of DNA PAINT, each of the four bases is labeled with a different oligo (binding partner 1) to which a complementary oligo (binding partner 2) transiently binds. Each of the four-nucleotide bases are associated with binding partner pairs of different sequence complements. In order to be differentiated the binding partner 2 associated with each of the four bases is distinguishable from the other. The element that makes them distinguishable can be a different wavelength emitting label (e.g. Atto 488, Cy3B, Alexa 594 and Atto 655/647N), labels with different lifetime or it can be that the different pairs are designed to have different on/off binding kinetics.

As well as resolution such methods can be used to precisely assign coordinates of localization of the signals. Localization is easier to determine when the fluorophore emitting the signal remains close to the site of incorporation, therefore the length and degree of flexibility of the linker or bridge joining the wavelength emitting moiety (e.g. fluorophore) to the base must be constrained, e.g. it is better to have a short length and a stiff linker.

The DNA PAINT also has the advantage that the fact that fluorophores photobleach is not of concern because they are always replaced by fresh imager strands. Therefore the choice of fluorophore, the provision of antifade, redox system is not that important and a simpler optical system can be constructed, e.g. without an f-stop to prevent illumination of molecules that are not in the field of view of the camera, because illumination only bleaches labels that transiently come into the evanescent wave.

Another alternative means to obtain a super-resolution image is by expansion (Chen, Tillberg, and Boyden Science 30 January 2015: Vol. 347 no. 6221 pp. 543-548).Here the elongated polynucleotide is rendered in a gel which is then expanded thereby stretching out the biological material. Specific labels associated with the polynucleotide are covalently anchored to the swellable polymer network. Upon swelling even if the polynucleotide is broken (and in other cases where the polynucleotide is broken or no longer has a contiguous polyphosphate backbone), the order of fragments is retained and the invention can still be practiced.

A number of approaches to obtain super-resolution, hardware based, chemistry based and algorithm based exist. In some embodiments the stretched polynucelotides are imaged via Scanning probe microscopy, transmission electron microscopy (Payne et al, PLoS ONE 8(7): e69058 (2013), scanning electron microscopy or Secondary Ion Mass Spectrometry (Cabin-Flaman et al Anal Chem. 83:6940-6947 (2011).

Virtual Super-Resolution and Super-Localization

When two or more origins are too close together for their signals to be optically resolvable (e.g. 50 nm from each other), the signals will appear to emanate from the same point source. When different bases are added at each of the origins a mixed signal representing the wavelengths of emission corresponding to each of the bases is obtained at the point source. It is difficult to determine which origin within the diffraction limited spot each of the signals emanates from. When the second and subsequent nucleotides are added to each of the origins, the sequence at each individual origin is hard to determine, as it is hard to deconvolve which sequence (extending from an origin) each of the signals corresponds to.

Extra cost, effort time is needed to implement one of the super-resolution methods described above. However, when a reference sequence is available, a solution to this problem is possible, as follows.

The methods described in this disclosure obtain multiple reads and provide the relative locations of the multiple reads on a single polynucleotide. Once a partial read (in some cases even when one base) has been obtained from a plurality of locations, then the reads and the distance separating them can be used to identify the location in a reference genome to which the polynucleotide aligns. This is similar to the matching between single DNA molecules and a reference that has been described in Marie et al (PNAS 2013). This allows one to see which part of the genome is being sequenced and therefore based on the reference it is possible to predict the sequence reads that would be expected at each of the locations. From this, the signals from multiple wavelength emissions that emanate from each non-resolved point source can be ascribed to a one or other sequence read expected within the non-resolved point source. As well as resolving closely spaced signals such a methods can precisely localize the signal too.

It is possible to use one or more reference sequences to predict what sequences should be present within the diffraction-limited spot carrying multiple mixed sequences. The task is easier when some of the reads are at resolvable locations (and so are not convoluted). Where none of the locations are resolved and continuum of signal along the polynucleotide is obtained, the sequence can nevertheless be resolved by tracking the signals that occur on each individual pixel and comparing against the reference.

If the sequence obtained from one of the origins, has a mutation, then it will show up as a different emission wavelength signal than expected from the reference but in a background of signals obtained through the cycles that mostly match the reference. In one in four occasions this mutation could correspond to sequence emerging from one or other of the origins, but if the other wavelength signals within the unresolved spot are as expected then the sequences can be probabilistically assigned. It is very unlikely that mutations would have occurred simultaneously at two or more locations at the same distance away from each origin. It is possible to know whether SNPs are present at both locations, and if so then the possible alleles. The alleles can also be resolved based on haplotype that are determined over the regions and by taking ethnic origins of the sample into account.

When the ethnic origin is not known or in the case, where the parents of an individual being sequenced are from different ethnic groups, then it is not straightforward to assume the probability of SNP alleles present in the location under analysis.

However, in some embodiments of the invention, the ethnic origins of each part of the genome, is determined orthogonally. For example, the genome can be analyzed using SNP arrays such as those available from Illumina and Affymetrix and the ethnic identity assigned to different parts of the genome based on the SNP data, can be used to determine which ethnic reference to use for a particular part of the genome.

With the benefit of a reference genome, or other copies of the genome with which to corroborate sequence, it is possible to assign signals from multiple origins that are unresolved, to specific origins. While this makes use of the reference genome to assist in making probabilistic determinations of base sequence in difficult to resolve instances, it does not rely on a reference genome for the structure of the genome. Therefore the principle reason the embodiments of this invention are of utility is retained.

The methods described in this section, are termed “Virtual” super-resolution, because the resolution is not through physics, but through bioinformatics. The virtual super-resolution methods can be combined with actual super-resolution methods to further increase the confidence in an assignment.

Merging Sequence Fronts

The aim of sequencing by coalescence is to obtain continuous sequence reads spanning two or more adjacent sequence fragments. To compile such a continuous read the sequence fragment from an origin must reach a downstream origin from where a sequence fragment has also been generated.

The individual reads must be of such a length that reads from a certain portion or fraction of individual origins are long enough to reach a downstream origin. The threshold fraction is defined herein as the portion of the overall number of reads that should go as far as an adjacent downstream origin; this may differ depending on the application. In single cell sequencing, where according to the aspects of this invention no amplification is performed, there is usually only one distinct copy of a genome (comprising 23 pairs of homologous chromosomes; each chromosome of the pair is distinct). In this case the threshold fraction needs to be very high, ideally all the upstream origins should go as far as to reach a downstream origin. But in cases where there are many copies of the genome, depending on the number of copies and the complexity of the genome, a substantially lower fraction will suffice. For example, a threshold fraction of one fifth of the genome can be sufficient, when the complexity is high (e.g. there is little repetitive DNA). This can be the case even in human genomes, when the aim is to derive information from genic regions such as exons or just from a panel of cancer genes. Typically, such regions, are low in repeats compared to non-genic regions. The one fifth threshold fraction does not allow the complete genome to be sequenced from a single copy of the genome, but as multiple copies of the genome can be used (1 ug has ˜20,000 copies of the genome), a region not covered by coalescent or non-coalescent read can be found to be covered in another molecule by a coalescent or non-coalescent read. The genome or the genomic region can then be reconstructed based on reads from the multiple copies.

For a sequence read to reach a downstream origin, it may abut against the origin, go past the origin and even if it falls short by a few bases, it can sufficient if the length of the gap can be determined or estimated. Such gaps can either be filled in by reads obtained from other copies of the molecules or simply just assigned as ambiguous or “N” position.

Reaching the threshold fraction can require different read lengths, depending on the proximity of the origins. Where the imaging is diffraction limited, the origins must be spaced at a distance equal to or the diffraction limit (e.g. >half the wavelength of light). This kind of read length is more suited to stepwise SbS using unlabeled nucleotides (e.g. 454 sequencing can generate reads several hundreds of bases in length) or by conducting real-time sequencing (PacBio sequencing can generate on average 10, 000 bases in length). In the case of SbS using reversible terminators, read lengths of 250 to 300 are currently achieved, using Illumina chemistry. The spacing of origins so that 300 base read length could span them needs to be ˜100 nm and a resolution of 100 nm or below, beyond the diffraction limit of light is needed, but which is matched to a super-resolution method such as SIM.

The sequence reads from multiple locations can be on one or other strand of the native genomic polynucleotide.

In some embodiments the two strands remain as a double helix. In some embodiments, the two strands are separated before sequencing. In some embodiments the two strands are substantially separated but remain next to each other; this is the case when chemical denaturation is applied (e.g. using alkali) on molecules that are already stretched out and immobilized. In some embodiments one of the two strands is removed. In some embodiments the two strands are separated in solution and do not re-anneal to a significant extent before they are stretched out. In some embodiments after capture by one end, the other strand is degraded. This can be done for example when the molecule is tethered at one end, not allowing access to exonuclease enzymes, whilst the other end is available for the action of a 3′ 5′ or 5′ 3′ exonuclease, degrading one or the other strand.

When only one strand is present then all the sequence fronts run in the same direction. However, when both strands are present, then sequence fronts run on both strands both travelling from 5′ to 3′, hence going in opposite directions, due to the anti-parallel nature of the double helix. This occurs either when the polynucleotide is double-stranded or a double strand is denatured but the strands remain too close to each other to resolve them. In these cases the coalescence between reads occurs in two ways. The first is when the sequencing front from one origin reaches a downstream origin, from which a sequencing front has also been initiated. The second is when the sequencing front travelling on the sense strand reaches a sequencing front travelling on the anti-sense strand. In this case the sequences can coalesce by joining the sequence from one strand with the complement of the other strand, in other words, the sense sequence from one strand with the anti-sense sequence from the other strand. This can occur across the polynucleotide at multiple locations.

If the duplex is still in place there is the possibility of one sequencing front knocking of the other or both coming off. However, the polymerases can be replaced by others provided in solution and the sequencing fronts can re-start.

Algorithms for coalescence and genome assembly take this bi-directionality into account. In other cases which strand each read belongs to can be determined.

The direction of migration of the sequencing front tells us which strand is being sequenced, this can be determined by looking at multiple cycles and detecting shifts in intensities on the pixels covering the point source (preferably between 3 and 8 pixels cover each point source) and the center of the signal can be determined by looking at the point spread function. In one aspect, the read length for coalescence to occur, on average, is halved, but the resolution constraint remains, e.g. it is just as hard to resolve two sequencing fronts on opposite strands as it is to resolve sequence fronts on the same strands. In another aspect, if the opposite reads are allowed to run through each other then, sequence from both strands is obtained, hence reducing any ambiguity in base calls, reducing sequencing error, and increasing confidence in sequence reads.

The prior art does not show how long contiguous stretches can be created by coalescence of reads obtained from a single polynucleotide.

Computational Processing of Coalescent Reads

In various embodiments, the method further comprises (f) ascertaining and storing the positions of the first and second locations in a computer memory; (g) storing the position and identity of the differently labeled nucleotides incorporated into the first sequence fragment and the second sequence fragment in step (e); and (h) ascertaining when the first and second sequence fragments coalesce and assembling the stored identity of the differently labeled nucleotides, thereby sequencing the single target polynucleotide.

After the reads are obtained, there are two approaches to coalesce the reads.

The first is where the position of the end of the read from a upstream origin reaches or goes past the origin of a downstream read. The second is where there is an overlap of sequence (e.g. an upstream read, reads past a downstream origin) of sufficient length (e.g. 10 bases) then it is possible to coalesce the reads by finding the overlap between reads.

In various embodiments, the method further comprises computationally trimming an overlapping segment of adjacent sequence fragments.

In various embodiments, the method further comprises computationally trimming an overlapping segment of adjacent sequence fragments. In various embodiments, the method further comprises (f) repeating steps (c) and (d) until a threshold fraction of adjacent sequence fragments overlap and result in redundant sequence reads spanning two or more adjacent sequence fragments. In various embodiments, the method further comprises (g) identifying any inconsistencies in the redundant sequence reads as potential sequencing errors.

Sequence Quality: Minimizing Sequencing Error and Coverage Bias

All sequencing technologies are subject to some level of error, and different sequencing platforms are susceptible to different kinds of error. According to Melanie Schirmer et al. (Nucl. Acids Res. 2015;nar.gku1341)1, Illumina Miseq raw error rates are 1 in 50. This includes errors introduced by library prep, cluster amplification, prephasing (errors in early incorporations), and phasing (error in the later incorporations). This can be reduced by trimming and overlapping reads to build a consensus, to ˜1 in 1000 or 99.9%.

In embodiments of this invention where no PCR is conducted, there is no coverage bias introduced due to PCR and there are no errors due to polymerase misincorporation during PCR. In Illumina, ABI SOLID, Ion Torrent, Intelligent Biosystems and Complete Genomics sequencing, PCR errors can be introduced during library preparation and during clonal amplification (e.g. DNA nanoball, polony or cluster generation).

However, once the clonal amplicons have been created, sequencing the amplicons in a bulk SbS reaction using reversible terminators plus polymerase or oligos plus ligase creates an aggregate read from many molecules, which swamps out signal due to incorporation error. In some embodiments of the present invention, clonal amplification, segment by segment, is performed on the elongated polynucleotide. This allows the single stochastic occurrence of a polymerase error to be outnumbered by a plurality of other polymerases acting on the amplicons (see below). As the presence of the reads can be detected directly on the original elongated single molecule, any drop in coverage (e.g. due to inefficiency of PCR in certain sequence context) can be directly observed visually or in post-processing of the data.

Another means for overcoming error in next generation sequencing is to carry out the sequencing on multiple copies of the unamplified genome in order to obtain reads of the same segment of the genome from multiple separate (non-amplicon) copies of the genome. The sequence is then assigned from a consensus of the many molecules. If two sequences are predominant, it may indicate heterozygosity. This is not an option when sequencing is done on a single cell. It is also problematic when the tissue or cell from which the multiple copies are obtained is not homogeneous. For example within a tumor there can be multiple clonal populations intermixed and somatic mutations may be present. The genomes are also altered in immune cells and direct single cell sequencing is needed. The methods of the invention are applied to such cases on a single polynucleotide basis, where high-levels of read coalescences preferred.

In some applications it is important to detect the somatic mutations that may have occurred in a population of cells. In this case it is better not to rely on being able to prune out error by obtaining consensus reads from many molecules, as it might be difficult to differentiate error from true rare mutations. Another problem with this is that the different copies may be paralogous, in that they are from different duplicons of a segment of the genome (segmental duplications), but which may contain small differences.

Error during sequencing also depends on the polymerase that is used. Polymerases with low error rate include Pfu, Pwo, and Fusion polymerases which have between 10⁻⁵-10⁻⁶ error rate and on average ˜2.5×10⁻⁶ error rate.

The sequencing errors due to polymerase incorporation error can however be pruned out by obtaining multiple reads over the region, without amplifying the region. This can be done by an upstream front reading past a downstream origin (‘Read-through’) and thereby creating a read redundancy. This can also be done by seeding multiple rounds of origin creation followed by SbS, which “reads-over” territory already covered, as well as new territories.

The sequencing errors due to polymerase incorporation error can also be pruned out carrying out sequencing over the same region on the polynucleotide multiple times. For example when the extension is done by an RNA polymerase (Sequencing by transcription), polymerases can load onto promoters multiple times and thereby a sequencing read can be occurring simultaneously with polymerases that are acting upstream and/or downstream of a given RNA polymerase. An erroneous incorporation can thus be pruned out according majority rules. Multiple reads can also be generated by removing the nucleotides that have been added by the polymerase and repeating the template-directed synthesis (using methods described below).

When sequencing is being done on single molecules, without amplification, error due to polymerase mis-incorporation can be overcome by methods that include testing the nucleotide to be incorporated multiple times before incorporation occurs This can be done by using a polymerase containing 3′ to 5′ exonuclease activity and tuning the concentration of nucleotides to be incorporated; so that before the extension proceeds to the next bases a base signal has been tested multiple times. The polymerase can be prevented from chewing back more than one nucleotide by providing a mixture of two types of nucleotides; the regular labeled sequencing nucleotides are supplemented with a phosphorothioate (e.g., a triphosphate analog with a phosphorothioate in place of the alpha-phosphate of the triphosphate chain, thereby preventing processive 3′ to 5′ exonuclease activity of polymerase) so that after several single base exonuclease excisions, a phosphorothiate nucleotide is incorporated, which cannot be removed by the exonuclease activity of the DNA polymerase. The several incorporations and removals, can include incorrect incorporations, but these will typically be outnumbered by the correct incorporations. The phosporothioate nucleotide does not need to bear a fluorophore and a cleavage cycle to remove a fluorophore is not needed. If the nucleotide does not bear a 3′ terminator, no cleavage is needed. The modification on the base can act as the terminator. Where termination is not complete and multiple nucleotides get incorporated, they can also be chewed back several times. This method can also be conducted in real time, as no cleavage mechanism is used. The ratio of labeled nucleotides to unlabeled phosphorothioate nucleotides determines the duration of each incorporation step. This multiplied testing for the correct base can also be done via methods described in Hoser (WO/2004/074503). These methods, share with the DNA PAINT mechanisms described herein, the ability to be superesolved, because labels in a closely packed field do not fluoresce at exactly the same times.

Thus in some embodiments the method comprises:

Using a polymerase with 3′ to 5′ exonuclease activity to incorporating a nucleotide bearing a terminator and label on the base, said label reporting on the identity of the base incorporated.

Using a polymerase with exonuclease activity so that a base is removed and another base is added, multiple times (because the switch from polymerase to exonuclease activity is triggered).

Providing a low concentration (or same or higher concentration but with lower incorporability) of unlabeled phosphorothiate nucleotide so that when it is incorporated it cannot be removed, thereby shifting the register to read the next nucleotide in the target polynucleotide

Repeating (i) to (iii) and thereby sequencing the polynucleotide.

In some embodiments, the above is carried out as a homogenous, single pot, real-time reaction. The shift from one base to the next can be a long-time (long enough to image multiple locations on the image plane) if the ratio of phosphorothiate nucleotide to fluorescent reversible terminator is low.

If a terminator, to prevent more than one nucleotide being incorporated is provided on one 2′ or 3′ of the sugar ring, a DNA repair enzyme such as Endouncuclease IV can be used or an exonuclease can be used to remove the whole of the nucleotide.

When sequencing on single molecules via detecting the incorporation of individual nucleotides, if the nucleotide is labeled with a single dye molecule as is done in Helicos and PacBio sequencing, errors can be introduced due to the dye not being detected. This can be because the dye has photobleached, the cumulative signal detected is weak due to dye blinking, the dye emits too weakly or the dye enters into a long dark photophysical state. This can be overcome in the present invention by two ways. The first is to label the dye with robust individual dyes that have favorable photophysical properties (e.g. Cy3B). Another is to provide buffer conditions and additives that reduce photobleaching and dark photophysical states (e.g. beta mercaptoethanol, Trolox, Vitamin C and its derivatives, redox systems). Another is to minimize exposure to light (e.g. having more sensitive detectors requiring shorter exposures or providing stroboscopic illumination). The second is to label with nanoparticles such as Quantum dots (e.g. Qdot 655), Fluorospheres, Plasmon Resonant Particles, light scattering particles etc. instead of single dyes. The third is to have many dyes per nucleotide rather than a single dye. In this case the multiple dyes may be organized in a way that minimizes their self-quenching (e.g. using rigid nanostructures, DNA origami that spaces them far enough apart) or a linear spacing via rigid linker. Genovoxx were able to incorporate nucleotides containing many fluorophores, Mir (WO2005040425) have been able incorporate nucleotides to which nanoparticles are attached. A fourth is to use DNA PAINT as described in this invention. Here the readout during the imaging step is obtained as an aggregate of many on/off interactions of different fluor bearing binding partners so even if one fluor is photobleached or is in a dark state, the fluors on other imager binding partners that land on the binding partner linked to the nucleotide may not be photobleached or in a dark state. A fifth is the exo digestion/phosphorothioate nucleotide approach described above. A sixth is the use of a nucleotide bearing multiple binding sites for imager strands which bind on and off simultaneously, giving a very bright signal, but without super-resolution. In contrast to the imager strands used in DNA PAINT, when multiple binding sites per nucleotide are used the binding of the imager strands can have a stability that provides long-lasting binding and hence signal, without the imagers rapidly coming off. The imager binding sites can be contiguous or can be separated by a nucleotide sequence or linker. The intervening nucleotide sequences can be made double stranded prior to the imaging reaction. In some embodiments when the aim is not to do super-resolution imaging, the long-lived imager strands can be bound to the nucleotides before the nucleotides are incorporated.

The detection error rate is further reduced (and signal longevity increased) in the presence of one or more compound(s) selected from urea, ascorbic acid or salt thereof, and isoascorbic acid or salt thereof, beta-mercaptoethanol (BME), DTT, a redox system, Trolox in the solution.

In real time sequencing where the dye is on the leaving group, the incorporation may be too fast for the frame rate of the camera and might not be detected. The incorporation rate can be slowed down by manipulating reaction conditions. Scientific CMOS cameras (e.g. the Orga Flash4.0 from Hamamatsu) are also available where the frame rate is high and are more likely to detect fast incorporating nucleotides.

Errors can also be introduced due to incomplete termination that can occur when the terminators are poorly performing “virtual” terminators. The solution to this is to use extremely robustly terminating terminators but where the termination can however be reversed after incorporation of the single nucleotide has been detected.

Re-Originate, Re-Read

Repeating Origination and Reading Multiple Time

In various embodiments, the method further comprises (f) seeding a second plurality of separately resolvable origins of polynucleotide synthesis along the single, elongated target polynucleotide molecule; (g) contacting the target polynucleotide molecule with the polymerase labeled nucleotides; (h) incorporating the labeled nucleotides, using the polymerase, into a second plurality of sequence fragments complementary to the target polynucleotide molecule and originating from the second plurality of separately resolvable origins of polynucleotide synthesis; (i) identifying and storing the identity and positions of the labeled nucleotides incorporated into each of the second plurality of sequence fragments, thereby determining the sequences and relative positions of the second plurality of sequence fragments; (j) repeating steps (h) and (i)

until a second threshold fraction of adjacent sequence fragments merge and result in continuous sequence reads spanning two or more adjacent sequence fragments; and (k) combining the sequence reads from steps (e) and (j), thereby sequencing the target polynucleotide molecule.

Seeding a plurality of separately resolvable origins of polynucleotide synthesis along the single, elongated target polynucleotide molecule and carrying out SbS can be repeated as many times as necessary to obtain the coverage and redundancy of sequencing required.

The practitioner of the invention has two options for obtaining reads for coalescence to take place. Either the read length is long enough to span from resolvable origin location to the next or the read lengths are shorter but are originated multiple times (each pass of sequence relates to each origination). Each time the reads are originated, they start from new random sites, and therefore one pass of sequencing the sites will be different from another pass of sequencing. So where in the first pass the read only reaches halfway to the next origins, the second pass may seed a read the starts at the halfway point and travels all the way to what was the second origin in the first pass. The advantage of this approach is that when it is repeated several times, the sequence of the polynucleotide may be covered several times over and if a genome is being sequenced multi-fold coverage can be obtained from the same DNA molecule.

Erase and Re-Read

In various embodiments, the method further comprises (f) degrading at least a fraction of the plurality of sequence fragments; and (g) repeating steps (c) and (d), thereby sequencing the plurality of sequence fragments.

In various embodiments, a 3′ to 5′ exonuclease is used to degrade the fraction of the plurality of sequence fragments.

In various embodiments, the differently labeled nucleotides are degradable nucleotides

In various embodiments, the degradable nucleotides are 5′ amide modified nucleotides which incorporate to form internucleoside P3′-N5′ Phosphoramidate (P-N) linkage which are cleaved by mild acid (Wolfe, J L, et al Nucleic Acids Res., September 1, 2002; 30(17):3739-3747; Shchepinov, M. Se t al. Nucleic Acids Res., 29, 3864-3872). Such nucleotides can be efficiently incorporated into DNA by the Klenow fragment of Escherichia coli DNA polymerase. An example of such a nucleotide is a phosphoramidate nucleotide, e.g. NH2-dNTP or NH2-NTP. The resulting modified internucleoside bond can be specifically cleaved by chemical treatment such as mild acid treatment. This embodiment can be carried out during either RNA (Gueroui 2002) or DNA synthesis. Following detection, the labeled degradation labile nucleotide is replaced by a degradation resistant nucleotide in order to shift the register to the next position in the sequence. This approach can be carried out by primer mediated DNA synthesis or promoter mediated RNA synthesis. The nucleotides can be labeled by standard methods (e.g. see Hermanson, G T or Mitra 2003). When a labeled phosphoramidate nucleotide is a reversibly terminated blocked at the 3′ end, the chain can be extended by one such nucleotide. The chemical treatment is preferably mild. For example, the phosphoramidate bonds formed within the resulting polynucleotides can be specifically cleaved with dilute acetic acid, for example 0.1M.

In various embodiments, the degradable nucleotides are RNA and are cleaved by an RNAse and/or alkali. In various embodiments, the degradable nucleotides are RNA and further comprising the steps of: (f) degrading at least one of the degradable nucleotides to leave an abasic site or nick; and (g) repeating step (c) using the abasic site or nick as an origin of polynucleotide synthesis.

In the case of SbS by using transcription as the synthesis method, the RNA transcript does not need to be degraded. This is because the transcript does not remain attached to the target polynucleotide during the entire course of its generation. To carry out SbS over the same region again, the promoter simply needs to be reloaded with an RNA polymerase again. In the case of transcription the RNA polymerase can be E. coli, T7, T3 or SP6 RNA polymerase. Abortive transcripts can be ignored or can be removed by de-stabillizing the complex.

In some embodiments where synthetic oligos are used for priming synthesis, the synthetic oligos can be RNA primers or DNA/RNA chimeric primers. In these embodiments the degradable RNA nucleotides are part of the primer. The RNA can then be degraded allowing the extended chain to be destabilized and easily removed and polymerization to be re-set.

In some embodiments where synthetic oligos are used for priming synthesis, the synthetic oligos and the extended nucleotides therefrom can be denatured from the polynucleotide and be flushed away. This is easily done when the target polynucleotide is stuck to the surface or in a gel or is disposed in a fluid flow.

Read Aggregation by Array Capture

In another embodiment capture reagents targeting specific polynucleotides or specific segments of polynucleotides are disposed on a surface or in a matrix are used to capture the target polynucleotides. In some embodiments the capture probes are designed to target certain generic sequences present on all polynuclotides in a sample. For example, an oligo (dT) capture reagent would target all RNA. In some embodiments, a common oligo sequence is grafted on to the target polynucleotides, so that they can be captured. Different capture reagents can be used to capture different polynucleotides, and the different capture reagents can be disposed in a spatially addressable ordered array such as a microarray. Once the polynucleotides are captured they can be elongated by fluid flow or electrophoretic flow.

Sequencing in a Flow Channel by Repeated Sample Refresh

An ultra-long polynucleotide (e.g. whole DNA from a chromosome) can enter a nanochannel (Frietag et al Biomicrofluidics 9:044114) using electrophoretic, fluidic and/or entropic forces. Many origins can be created before or after the polynucleotide is disposed in the channel and SbS including real-time sequencing can be conducted, while the polynucleotide is held suspended within the channel, until a threshold fraction of reads coalesce. Once the molecule is sequenced, it is optionally flushed out of the channel and the next polynucleotide is added. An advantage of not immobilizing the polynucleotide on a surface is that the reaction kinetics are more rapid and the interactions are not constrained by steric hindrance. In another embodiment a sample comprising RNA molecules are immobilized on a surface or matrix, sequenced by the methods of this invention, and then removed, before the next RNA sample is immobilized and sequenced. The RNA molecules can be removed by change of buffer or an extrinsic trigger, such as UV light for the cleavage of a photo-cleavable linkage via which the RNA is anchored to the surface or in the matrix.

Sequencing by Hybridization and Coalescence

In some embodiments of the invention, sequencing reads are not obtained per se. In the case of sequencing by hybridization, the read is the complement of the oligo which hybridized to a specific location on the polynucleotide. At the first level an assembly is done from sequence information gathered by hybridization of oligos. Thus some embodiments of the invention comprise:

(i) Stretching the polynucleotide (s)

(ii) Denaturing the polynucleotide (s) (removing secondary structure if the target is RNA, separating the double helix when the target is double stranded DNA, e.g. Genomic DNA)

Hybridizing short oligos so that they remain stably attached to the target

Determining location of binding of the short oligos

In some embodiments, each oligo sequence is added one at a time.

In some embodiments the oligo bears a tag from which its identity can be decoded, e.g. a sequence tag, for example to which an orthogonal set of oligos can be bound or on which SbS is done to determine its identity. In some embodiments more than one oligo is added at a time. In some embodiments as many oligos as can be decoded are added. For example if 16 distinct codes are available, 16 oligo sequences each bearing one of the codes are added simultaneously. In some embodiments substantially more oligos are added and distinguished by using optical barcodes such as DNA origami (Nat Chem. 2012 Oct;4(10):832-9). In some embodiments a complete set of oligos, e.g. every 5 mer or 6 mer is used.

In some embodiments Toehold probes (Nature Methods 10: 865 (2013)) are used comprising partial double strand that is competitively destabilized when bound to a mismatching target. This method can ensure the accuracy of sequencing by hybridization. The method comprises:

(i) Stretching a polynucleotide

(ii) If the polynucleotide is not single stranded, making it substantially single-stranded

Hybridizing a repertoire of toehold probes to the target polynucleotide

Determining location of binding of hybridized oligo from the toehold for each toehold in the repertoire

Reconstructing the sequence based on the hybridization localization data for all the sequences in the repertoire

The short-range sequence within the diffraction-limited spot is assembled based on oligos or toehold probes that fall within the spot. The long-range sequence is assembled by coalescing the sequence assembled from adjacent or overlapping spots.

In some embodiments rather than using a flow cell, when the polynucleotide is attached to a surface, the surface (e.g. coverglass) is dipped into different troughs carrying different reagents (e.g. oligos, alkali) of the reaction.

In some embodiments, following hybridization the oligo acts as a primer to initiate SbS. In some embodiments the oligo repertoire acts as random primers. In some embodiments oligos are designed to be complementary to specific parts of the genome and are used to initiate selective sequencing by coalescence from those specific parts of the genome.

Sequencing by Opto-Mechanical Read-Out

In some embodiments of the invention, a modified version of the system described by Ding et al (Nature Methods 9, 367-372 (2012)) is used. In this embodiment a hairpin is ligated to one end of a target double stranded template, and a biotin is added to one strand of the other end and Digoxygenin (DIG) to the other strand of the other end. The polynucleotide is immobilized via the DIG and a paramagnetic nanoparticle is attached to the Biotin end. A magnetic tweezing system is then used to pries the duplex apart by translating the magnetic field in the Z direction with respect to the stage holding the anti-DIG coated surface while the sense and antisense strands remain connected through the sequence of the hairpin. This leads to elongation of the single strand vertically from the planar surface. Then ligands (e.g. oligos) are allowed to bind events along the length of the sense/antisense polynucleotide. The precise location of binding of each of the ligands is then determined by making optical measurements of the paramagnetic bead, as the polynucleotide is allowed to re-nature. The vertical position of the magnetic bead is detected by imaging (on a CCD or CMOS camera) the size of the bead image, which becomes smaller or enlarges depending on its distance from the focal point.

The present invention implements this concept on long polynucleotide fragments, including complete RNA transcript lengths and long (>40 Kb tracts of genomic DNA) and provides a mechanism for sequencing the polynucleotide.

The SbS or sequencing by hybridization reactions of this invention are initiated along the length of the sense/antisense polynucleotide. In some embodiments, very short oligos, such as 3, 4 or 5 mers are hybridized so that the number of oligos in the repertoire (hence the number of hybridization cycles) is small.

After hybridization has occurred to the locations along the polynucleotide to which the oligos have bound (that contain a complement to the particular oligo added) are detected. This is done by turning off the magnetic tweezing and allowing the separated strands (that are linked by the hairpin) to reform and as the duplex is reformed all the bound oligo are displaced and ejected. Every time an oligo is displaced by the reformation of the native duplex, there is a pause detected. Then another set of primers or oligos can be added. Alternatively, a set of anti-methyl or anti-hydroxymethyl antibodies (as well as antibodies to other modification) antibodies can be added and their location detected. Unless the precise length or identity of the polynucleotide is already known, the antibody binding information needs to be coupled on the same polynucleotide with sequence information.

The formation of a duplex with a 3 mer has the advantage that there are only 64 varieties so at most only 64 cycles are needed. The binding of such short oligos however, requires very low temperatures as studied by Olke Uhlenbeck (J Mol. Biol. 1972, 65:25-41) as well as high salt and optionally, divalent cations. The precise location of the 3 mers can then allow the sequence to be assembled by coalescence of the 3 base reads. The 3 mer repertoire can also be supplemented with a few longer oligos. Also the stability of the 3 mer can be increased by using modified nucleotides such as LNA or PNA nucleotides, by attaching thereunto stabilization moieties such as spermine and/or by the addition of additional degenerate or universal base positions, for example the oligo may comprise a 3 base specific sequence with 5 base universal sequence.

In one embodiment an RNA polynucleotide is sequenced. This is done via cDNA synthesis followed by second strand synthesis using AMV reverse transcriptase which creates a hairpin between the first and second strand. The primer can be biotinylated and can be attached to a surface via the streptavidin. The non-attached end can then be attached to DIG then a magnetic bead in order to conduct opto-mechanical sequencing. One advantage of this approach is that if a mismatch hybridization has occurred it can be distinguished from the perfect match by a difference in the pause that is detected.

In order to make measurements on long polynucleoitdes, e.g.>50 Kb and going towards megabases, the polynucleotide is not stretched perpendicular to a surface but is instead stretched at an oblique angle from the surface and in some cases virtually parallel to the surface. In this case, the change in image of the bead is different to the perpendicular case but can be calculated. In some embodiments where the polynucleotide is stretched parallel to the surface, the lateral displacement of the bead is detected.

In some embodiments the hairpin structure is used in a different sequencing mechanism, which for example sensitively determines subtle differences in the re-folding state of the hairpin. For example, the more compact and dense structure of the hairpin can be used to as a capacitor, in a system where the surface is electronically connected.

In some embodiments, information from multiple rounds of hybridization with different oligos or groups of oligos is integrated to re-construct the sequence of the polynucleotide.

One advantage of the approach is that the stability of the duplex will be affected by mismatching, so it will be possible to distinguish a mismatch from a perfect patch. A second advantage is that it will be possible to test multiple oligos at the same time—as long as the stability of the duplexes formed by oligos are different then it will be possible to distinguish them.

So in one embodiment of the invention, opto-mechanical coalescent sequencing on the hairpin system comprises:

Separating short oligos into minimally overlapping groups, where each oligo in the group binds to the polynucleotide single strand with different stabilities.

Opening the duplex to make a contiguous sense/antisense single stranded target

Adding one group to the sense/antisense single stranded target

Allowing the duplex to reform whilst recording the location of the oligo and the force required to remove each oligo (where the force does not correspond to an expected value, it can be surmised that a mismatch may have occurred, and the data point is ignored

Optionally repeating the opening of the duplex and oligo binding multiple times to increase confidence, as desired

Exchanging reagents and adding the next of group and repeating 1-5

Deconvolving the oligo identity from the force data and using the oligo identity and its location information to assemble the polynucleotide.

Making Sense-Antisense Single Strands for Sequencing

Similar to the opto-mechanical sequencing described above, in some embodiments, a hairpin is ligated onto an end of a double stranded template and one of the other ends is immobilized on a surface via only one of the strands. The polynucleotide is then denatured and elongated/stretched out parallel to the surface of attachment. The polynucleotide is then fixed in the elongated state.

This provides a way to ensure that the target is single stranded and it is known that reads obtained from one of the two ends. Further the reads obtained from the end-on-end sense and antisense strands provide complementary reads, which is an internal validation of the verity of the sequencing obtained. Origins for sequencing can be created by annealing oligos to the sense and antisense single strands. Such sense-antisense strands can also be made by doing cDNA synthesis on RNA using AMV reverse transcriptase which naturally makes a hairpin to synthesize a second strand. In this case the primer for reverse transcription can me modified with a moiety that allows attachment to the surface.

Similarly, segmental amplification of the sense/antisense strand can be conducted. In some embodiments the hairpin sequence can contain the primers for PCR. In some embodiments the hairpin templates for sequence methods of the present invention and of Ding et al, can be created by Tagmentation mediated insertion and fragmentation. One oligo in the transposase complex can be modified for immobilization and the other can be a hairpin.

Integrating Reads From Multiple Polynucleotides

Preferably the contiguous sequence is obtained via de novo assembly. However, the reference sequence can also be used to facilitate assembly. This allows a de novo assembly to be constructed but it is harder resolve individual haplotypes of very long distances, enough locations need to be encountered along the molecule that are informative about the haplotype. When complete genomes sequencing requires a synthesis of information from multiple molecules spanning the same segment of the genome (ideally molecules that are derived from the same parental chromosome, algorithms are needed to process the information obtained from multiple molecules. One algorithm is of the kind that aligns molecules based on sequences that are common between multiple molecules, and fills in the gap in each molecule by imputing from co-aligned molecules where the region is covered. So a gap in one molecule is covered by read in another (co-aligned molecule). Further, shotgun assembly methods such as that developed by Eugene Myers can be adapted to carry out the assembly, with the additional advantage that a multitude of reads are pre-assembled (e.g. it is already known the location of reads with respect to each other, the length of gaps between reads is known). Other algorithmic approaches such as the SUTTA by Mishra et al (Bioinformatics, Oxford Journals, (2011) 27 (2): 153-160) can also be adapted for assembly of the data. In various embodiments, a reference genome can be used to facilitate assembly, either of the long-range genome structure or the short-range polynucleotide sequence or both. The reads can be partially de-novo assembled and then aligned to the reference and then the reference-assisted assemblies can be de-novo assembled further. Various reference assemblies (e.g. from different ethnic groups) can be used to provide some guidance for a genome assembly, however, information obtained from actual molecules (especially if it is corroborated by two or more molecules) is weighted greater than any information from references. The prior art does not show that a contiguous sequence can be reconstructed by aligning locational sequence obtained from a plurality of individually examined polynucleotides.

Sequencing Without a Reference

In various embodiments, the sequence is determined without using another copy of the target polynucleotide molecule or reference sequence for the target polynucleotide molecule. In this case the most of the reads (e.g. 90%) will have coalesced and the gap between reads of those reads that have not coalesced will be known. The gap distance will be known because the linear length of the polynucleotide will be traceable and the gap distance can be determined by counting the number of pixels between reads, and using knowledge of the length of DNA each pixel spans.

Haplotype Resolved Sequencing

Genomic sequence would have much greater utility if haplotype information (the association of alleles along a single DNA molecule derived from a single parental chromosome) could be obtained over a long range.

In various aspects and embodiments, the methods can be used for sequencing haplotypes. Sequencing haplotypes can include the steps of sequencing a first target polynucleotide spanning a haplotypic branch of a diploid genome using a method according to the invention; sequencing a second target polynucleotide spanning the haplotypic branch of the diploid genome using a method according to the invention, wherein the first and second target polynucleotides are from different copies of a homologous chromosome; and comparing the sequence of the first and second target polynucleotides, thereby determining the haplotypes on the first and second target polynucleotides.

Determining Haplotype Diversity and Frequency In A Cell Population

In many existing methods where the aim is to look at the heterogeneity of genomes in a population of cells, single cell analysis is used which is technically demanding. However, a remarkable feature of the present invention is that the heterogeneity of genomes in a population can be analyzed without the need to keep the content of single cells together because if molecules are long enough one can determine the different chromosomes, long chromosomes segments or haplotypes that are present in the population of cells. Although this does not indicate which two haplotypes are present in a cell together, it does report on the diversity of genomic structural types (or haplotypes) and their frequency and which aberrant structural variants are present. This embodiment comprises the steps:

Extracting genomic DNA from two or more cells

Elongating the DNA and carrying out a sequencing method of this invention

Analyzing the data to determine which DNA strands are homologs

Determining the different haplotypes among the homologs

Determining the frequency of the different haplotypes.

Synergizing With Other Sort Read Sequencing Technologies

In some embodiments, the methods of this invention stop short of being a complete genome sequencing and are used to provide a scaffold for short read sequencing such as that from Illumina. In this case it is advantageous to conduct Illumina library prep by excluding the PCR amplification step to obtain a more even coverage of the genome. One advantage of some of these embodiments that fold coverage of sequencing required can be halved from about 40× to 20× for example. In some embodiments this is due to the addition of sequencing done by the methods of the invention and the locational information that methods provide.

Coalescence by Integration

In some embodiments in addition to abutting and overlapping reads, some reads are separated by gaps. These gaps are of varying lengths. The gap lengths can be measured accurately when single molecule localization methods are used to detect the distance between the incorporated bases emanating from nearest neighbor origins. In some embodiments some or all of the gaps can be filled-in by transmuting sequence from the reference. In some embodiments some or all of the gaps are closed by sequencing from new start sites. In some embodiments some or all of sequence in the gaps is reconstructed from other molecules, which do not contain the same gaps, i.e. a second molecule has sequence over the region that a first molecule has a gap (see FIG. 10).

Here, the genome is extracted from multiple cells and therefore many copies of the molecule is present on the surface; the results from the same homologs are collected and a consensus read is obtained; homologous molecules are separated, to provide a haplotype or parental chromosome specific read.

Starting with around lug of genomic DNA, if there are a thousand start sites over each of the megabase length molecules, and they are on average 1 Kbp apart. Then out of the thousand 25-60 base reads, a few reads from one molecule will overlap with a few from another molecule, and this will allow us to align the two megabase fragments and depending on where they stitch together, the overall length will be extended and in the overlapping regions the reads that were only found on one of the strands will fill some parts of the gaps in the other molecule. The same will happen with other molecules of the ˜20,000 copies of the genome, until all or most of the gaps are filled.

Sequencing Panels

In some embodiments, it is desirous to sequence a subset of the genome corresponding to specific genes or loci. In this case, the genomic DNA is made single stranded and a sequence-specific primers are annealed over the regions of interest and SbS is conducted to obtain sequence reads and preferably coalescent reads. One advantage of targeting the sequencing in this way, is that even if the whole of the genome is stretched onto the surface, only the targeted regions light up. So imaging time can be shortened by going directly to the light detectable target regions. Furthermore, the genome can be arrayed on the surface at a much higher density than normal, because only a small sub-fraction of the molecules need to be detected. As an example, the BRCA1 region of the human genome can be sequenced by annealing a plurality of primers complementary to BRCA1 sequences and carrying out SbS and obtaining coalescence.

Cell-Free Nucleic Acids

Some of the most accessible DNA or RNA for diagnostics is found extraneous of cells in body fluids or stool. DNA circulating in blood is used for pre-natal testing for trisomy 21 and other chromosomal and genomic disorders. It is also a means to detect tumor derived DNA. However the molecules are typically in the ˜200 bp length range in blood and shorter in Urine. The copy number of a genomic region is determined by comparison to the number of reads that align to the reference compared to other parts of the genome. The present invention can be applied to the enumeration of cell free DNA sequences by:

isolating cell free DNA from blood

concatenating DNA

performing sequencing by coalescence on the concatenated DNA

Catenation can be done by polishing the ends of the DNA and performing blunt end-ligation. Alternatively, the blood or the cell free DNA can be split into two aliquots and one aliquot is tailed with poly A (using Terminal Transferase) and the other aliquot is tailed by polyT. The two aliquots are then combined, annealed and any recess filled in by DNA polymerase and ligated. Methods developed for contatenation in Serial Analysis of Gene Expression (SAGE) can be used. In some embodiments where the polynucleotide is single standed, e.g. RNA, the molecules can be concatenating by using T4 RNA ligase. T4 RNA Ligase 1 catalyzes the ligation of a 5′ phosphoryl-terminated nucleic acid donor to a 3′ hydroxyl-terminated nucleic acid acceptor through the formation of a 3′→5′ phosphodiester bond.

The resulting concatamers are then subjected to sequencing by coalescence. The resulting “super” sequence read is then compared to reference to extract individual reads. The individual reads are computationally extracted and then processed in the same manner as other short reads.

DNA is also found in stool a medium that contains a high number of exonucleases which can degrade the DNA; high amounts of chelators (e.g. EDTA) of divalent cations, which are needed by exonucleases to function, can be employed to keep the DNA sufficiently intact and sequenced according to the methods of the invention. Another way that DNA is shed from cells is via encapsulation in exosomes. Exosomes can be isolated by ultracentrifugation or by using spin columns (Qiagen), the DNA or RNA can be collected and sequenced according to the methods of the invention.

RNA Sequencing

The lengths of RNA are typically shorter than genomic DNA but it is challenging to sequence RNA from one end to the other using current technologies. Nevertheless, because of alternative splicing splicing it is vitally important to obtain determine the full sequence composition of the mRNA. In some embodiments of the invention mRNA can be captured by binding of its polyA tail by immobilized oligo d(T), its secondary structure removed by stretching force and denaturation conditions so that it can be elongated on the surface. This then allows random primers, or sequence-specific (e.g. exon-specific) primer to bind and initiate SbS. Typically the same nucleotides as used for DNA templates can be used for cDNA synthesis by reverse transcriptases and certain DNA polymerases (e.g. Klenow) (Ozsolak et al Nature 461:844 (2009)). Because of the short length of RNA it is beneficial to employ the super-resolution methods described in this invention to resolved multiple origins of synthesis on RNA. In some embodiments just enough read length from origins scattered across the RNA is sufficient to determine the order and identity of exons in the mRNA for a particular mRNA isoform.

Sequencing Applications and Uses

In some embodiments the invention comprises uses of sequence information that is obtained from a single elongated polynucleotide directly or after the single elongated polynucleotide has undergone segmental clonal amplification, where the context of short (e.g. Illumina, Ion Torrent) or mid-sized (e.g. Pacific Biosciences) sequence reads within a long template polynucleotide (from ˜100 Kb to a whole chromosome) are preserved. The context information can just comprise the information that the short read originates from a particular polynucleotide. The context can also extend to knowing the precise or approximate location of the sequencing read within the polynucleotide.

Moreover, even longer range information than the length of an individual polynucleotide (if it is of sub-chromosomal length) can be obtained when the polynucleotide is part of a plurality of polynucleotides, of similar or different lengths that stem from the same chromosome (or other type of complete polynucleotide, e.g. an RNA transcript). In some embodiments, sequence reads from each of the polynucleotides in the plurality are obtained independently of reads from other polynucleotides that comprise the polarity of polynucleotides. In this case, the sequencing data obtained from the plurality of polynucleotides is used to reconstruct or assemble the polynucleotide into the native polynucleotide sequence from which the polynucleotides originally emanated. This can be the case when sequencing is done on genomic DNA extracted from many cells of a given type, and it is expected that DNA from many of the same chromosome homologs are present. For example, in cell extraction from one million cells, (e.g. a lymphoblastoid cell line from a CEPH panel, e.g. NA12878) one million chromosome 1 homologs derived from the mother and one million chromosome homologs derived from the father would be expected in the extracted DNA.

In other embodiments the context of the short reads is preserved by sequencing an isolated long (˜50-200 Kb) single polynucleotide. In some embodiments the context of the short reads are preserved by sequencing along an elongated polynucleotide. In other embodiments the context of the short reads is preserved by preparing a library from an isolated single polynucleotide, such libraries are then sequenced. In some embodiments many copies of single polynucleotide that cover the same segment (with or without haplotype resolution), are used as templates to obtain a plurality of sequence reads per template, and the sequence reads are used to reconstruct a longer range sequence of the polynucleotide segment than can be represented by one of the single polynucleotides. Hence a de novo assembly of a genome, or large parts of the genome can be reconstructed. In order to make a haplotype resolved de novo assembly, when a sufficient fraction of a polynucleotide is covered with sequencing reads, it is possible to differentiate overlapping segments as belonging to a segment from one homologous chromosome or another (e.g. based on SNPs or structural variants found therein). The methods of the invention can be used to determine or resolve the following features that can be found in a genome that are difficult to obtain by current sequencing technologies.

Inversions

The orientation of a series of sequence reads along the polynucleotide will report on whether an inversion event has occurred. One or more reads in the opposite orientation to other reads compared to the reference, indicates an inversion.

Translocations

The presence of one or more reads that is not expected in the context of other reads in its vicinity indicates a rearrangement or translocation compared to reference. The location of the read in the reference indicates which part of the genome may have shifted to another. In some cases the read in its new location may be a duplication rather than a translocation.

Copy Number Variations

The absence or repetition of specific reads indicates that a deletion or amplification, respectively has occurred. The methods of this invention can particularly be applied in cases where there are multiple and/or complex rearrangements in a polynucleotide. Because the methods of the invention are based on analysing single polynucleotides, the structural variants described above can be resolved down to a rare occurrence in small numbers of cells for example, just 1% of cells from a population.

Duplicons

Segmental duplications or Duplicons are persistent in the genome and seed a lot of the structural variation in individuals' genome including somatic mutations. The Segmental Duplicons, may exist in distal parts of the genome. In current next generation sequencing, it is difficult to determine which segmental duplicon a read arises from. In some embodiments of the present invention, because reads are obtained over long molecules (e.g. 1-10 Megabase length range), it is usually possible to determine the genomic context of a duplicon (simply by using the reads to determine which segments of the genome are flanking a particular segment of the genome) because the crux of the invention is that the location of the reads are known or can be determined once the data is analysed. This comprises the steps:

Repetitive Regions

The repeated occurrence of a read or related read carrying paralogous variation can be observed by the methods of the invention (after data analysis), as multiple or very similar reads occurring at multiple locations in the genome. These multiple locations may be packed close together, as in satellite DNA or they may be dispersed across the genome such as pseudogenes. The methods of the inventions can be applied to the Short Tande Repeats (STRS), Variable number of Tandem Repeats (VNTR), trinucleotide repeats etc.

Finding Breakpoints

Breakpoints of structural variants can be pinpointed by the methods of the invention. Not only does the invention show at a gross level, which two parts of the genome have fused, but the precise individual read at which the breakpoint has occurred can be seen. Not only does the read comprise a chimera of the two fused regions, all the sequences on one side of the breakpoint will correspond to one of the fused segments and the other side is the other of the fused segments. This gives high confidence in determining a breakpoint. Even in cases where the structure is complex around breakpoint, the methods of the invention can resolve the structure. In some embodiments the precise chromosomal breakpoint information is used in understanding of a disease mechanism, used in detecting the occurrence of a specific translocation and diagnosing a disease.

Haplotypes

In some embodiments the resolution of haplotypes enables improved genetic studies to be conducted. In other embodiments the resolution of halpotypes enables better tissue typing to be conducted. In some embodiments the resolution of haplotypes or the detection of a particular haplotype enables a diagnosis to be made.

Compared to other inferential or partition and tagging haplotyping/phasing approaches, the present invention is not based on computer reconstruction of a probable haplotype. The visual nature of the information obtained by the invention, actually physically or visually shows a particular haplotype.

Hence reads, coalescent reads and assemblies that are obtained from the embodiments of this invention can be classed as being haplotype-specific. The only case where haplotype-specific information is not necessarily easily obtained over a long range is when the threshold of coalescence is low or when there is no coalescence but the location of the reads is provided nonetheless. Even here, if multiple polynucleotides cover the same segment of the genome the haplotype can be determined computationally.

Identification of Organisms

One embodiment of the invention is to identify the different individual organisms present in a mixed sample such as metagenomic sample, based on the sequence, methylation and structural information provided by the invention. As sequencing by coalescence can sequence a substantial fraction of a genome from just one copy of the genome, it can sequence a diverse metagenomic mixture of s. Furthermore just the map of a single molecule obtained from one or a few bases of information is sufficient to identify an microorganism.

Cell Line Identification and Validation

In some embodiments, the genomic DNA is extracted from cells in culture, stretched out and methylation and/or sequence information is extracted from the stretched molecules using the methods of the invention. This information can be used to validate the identity of the cell line and to determine its molecular phenotype and to monitor changes in its (epigenome through the course of passaging or as experiments are preformed (e.g. perturbation of growth conditions).

Disease Detection

In some embodiment the invention comprises use of the methods of the invention for the early detection of cancer, diagnosis of cancer, classification of cancer, analysing the cell heterogeneity within cancer, staging the cancer, monitoring development of cancer, deciding whether to apply drug treatment, which drug or combination of drugs to use, monitoring the effect of treatment monitoring of relapse, prognosticating outcomes. In each of these cases, either a specific “biomarker” or set of biomarkers is looked for, which comprise a particular structural variant or just the occurrence of structural variation in general above a certain threshold level is detected. This aspect comprises:

Obtaining sample biomaterial from a human patient or an individual that is being screened (e.g. for early cancer detection)

Performing sequencing and/or methylation analysis according to the methods of the invention

Looking for sequence, methylation and/or structural variation in the data, compared to a reference or compared to other body tissue from the individual/patient

Assessing the amount and/or type of variation and optionally providing a score

Optionally making a clinical decision based on 4.

The same five steps can be applied to other disease cases than cancer and can be applied to animals other than humans, such as livestock, dogs and cats. The sequence data can include RNA and DNA data. In some embodiments only sequence, only structural or only methylation information is used to make the clinical decision.

In some embodiments step 5 can comprise deciding which fertilized egg to choose in pre-implantation diagnosis or screening.

Genotype to Phenotype Correlations

In some embodiments the methods of this invention are used to make genotype to phenotype correlations in

Obtaining sample biomaterial from individuals in a population, cohort or family

Performing sequencing and/or methylation analysis according to the methods of the invention

Looking for sequence, methylation and/or structural variants in the data and comparing it between cases and controls for a specific disease or trait whilst optionally taking ethnicities, stratification of phenotypes and misclassification of phenotype into account

Determining which sequence, methylation and/or structural motifs or markers correlate with phenotype

Obtaining candidate sequence, methylation and/or structural variant biomarkers for the phenotype according to 4

Optionally using the candidate information from 4 to define a biomarker or perform further studies to fine tune or validate the biomarker

### DETAILED DESCRIPTION OF EXPERIMENTS

As many of the required procedures are standard molecular biology procedures that lab manual, Sambrook and Russell, Molecular Cloning A laboratory Manual, CSL Press (www.Molecular Cloning.com) can be consulted. Also Eckstein, editor, Oligos and Analogues: A Practical Approach (IRL Press, Oxford, 1991) and M. J. Gait (ed.), 1984, Oligo Synthesis; B. D. Hames & S. J. Higgins (eds.) can be consulted for DNA synthesis. The following three handbooks provide useful practical information: Handbook of Fluorescent Probes (Molecular Probes, www.probes.com); Handbook of Optical Filters for Fluorescence Microscopy (www.chroma.com); Single-Molecule Techniques: A Laboratory Manual, Edited by Paul R. Selvin, University of Illinois, Urbana Champaign; Taekjip Ha, University of Illinois, Urbana-Champaign; Focus on Single Molecule Analysis, Nature Methods, June 2008 Volume 5, No 6. The embodiments within the specification provide an illustration of embodiments of the invention and should not be construed to limit the scope of the invention. The skilled artisan will recognize that many other aspects and embodiments are encompassed by the methods of this invention. The embodiments of the invention and technical details provided below can be varied by the skilled artisan and can be tested and systematically optimized without undue experimentation or re-invention.

Whether explicitly stated or not, all the mechanisms described herein can be repeated, for example multiple cycles (e.g. 10-750) of sequencing, each comprising essentially the same steps can be conducted, to achieve coalescence of reads

The methods of this invention comprise various wash steps in between the main functional elements of the process, the need for wash steps at various points will be recognized by the skilled artisan. In general the wash puffer can comprise, Phosphate Buffered Saline, 2×SSC, TE, TEN, HEPES and may be supplemented with small amounts of Tween 20, Triton X. Sarkosyl, and/or SDS. Typically 2-3 washes can be inserted in between functional steps.

Various Illumina SBS kits (e.g., TrusSeq SBS Kit) can be used for sequencing with reagent addition and imaging in the following order: Universal Sequencing Buffer; Incorporation Mastermix; Universal Sequencing Buffer; Universal Scan Mix; Imaging Cleavage Reagent Mastermix; Cleavage Wash Mix. These regents are loaded into a flow cell carrying the templates to be sequenced. Details of the Illumina kit can be downloaded from the world wide website: https://supportillumina.com/content/dam/illuminasupport/documents/myillumina/6936f0c7-b8cb-4a62-bcc5-207a05850b1f/truseq_sbsv5_ga_reagentprepguide_15013595_d.pdf

Imaging is done by using 532 nm laser for two of the four dyes and 660 nm laser for the other two of the dyes on the nucleotides. Each of the two dyes excited by each laser is differentiated by using specific emission filters and an algorithm designed to determine the signatures of each dye.

One of a number of different Illumina sequencing instruments can be used including the Genome Analyzer IIx, which is particularly appropriate, as it comprises PRISM-TIRF and a fiber-optic scrambler. A flow cell footprint compatible with the Illumina flow cell holder and inlet and outlet ports can be used. Alternatively, a home-built system comprising an inverted microscope, with high numerical aperture objective lens, lasers, CCD camera, fluorophore selective filters and syringe pump based or pressure driven reagent exchange system and a heated stage. The home-built system can be adapted for other nucleotide/dye combinations than offered by Illumina.

As an alternative to using chemically cleavable reversible terminators (as per the Illumina method), photocleavable nucleotides can also be used. Here the cleavage step includes shining of UV light as described below. A photocleavable 2-nitrobenzyl linker at 3′ end can be used as a photoreversible linker for a blocker and/or label. The photolabile linker can generally be cleaved by irradiation for 5-15 minutes with 300-360 nm light with gentle mixing, in a buffer of choice. In some embodiments the buffer used is one suitable for nucleotide incorporation by the polymerase that is used and is compatible with a homogeneous sequencing reaction that does not require exchange of reagents. In some embodiments the buffer of choice contains a salt concentration similar to Phosphate Buffered Saline. The addition of DTT in the buffer has a beneficial effect (Stupi et al. Angew Chem 1724-1727) and can speed up the reaction. For better efficacy specific protocols can be used. In one protocol photocleavage is achieved by UV light at 355 nm at 1.5 W/cm2, 50 mJ/pulse. One pulse is for 7ns and this is repeated for a total of 10 sec. Lightening terminators developed by Metzker and co-workers at Lasergen Inc, are highly favorable photocleavable nucleotides. These nucleotides have a 2-nitrobenzyl group attached to bases that are hydroxymehtylated and are incorporated by Therminator with fast kinetics, allowing the incorporation reaction time to be short, e.g. down to a minute.

Imaging is done by using 532 nm laser for two of the four dyes and 660 nm laser for the other two of the dyes on the nucleotides. Each of the two dyes excited by each laser is differentiated by using specific emission filters and an algorithm designed to determine the signatures of each dye.

Extracting and Elongating Megabase Range Genomic DNA on a Surface

A number of methods exist for extracting and stretching High Molecular weight (HMW) or long length DNA. A Molecular Combing (Allemand et al Biophysical Journal 73:2064-2070 1997; Michalet et al Science 277:1518-1523 (1999)) protocol adapted from Kaykov et al (Scientific Reports 6:19636 2016) can be used to extract and elongate DNA with average lengths in the mega-base range. Genomic DNA is extracted from cells (1×10⁴ to 10⁵ per block) in agarose blocks (e.g. using Biorad or Genomic Vision protocol or as described by Kaykov et al) using Proteinase K for 1 hour, the washing step includes 100 mM NaCl, the agarose block is melted and digested in a trough using Beta-Agarase (NEB, USA) for an extended period (e.g. 16 hrs) at 42° C. without mixing and then brought to room temperature. DNA is combed in a buffer containing 50 mM MES 100 mM of NaCl at pH 6. A device that can pull a substrate (e.g. coverslip) out of a trough (e.g. as described by Kaykov) is used to generate smooth, low friction z movement with minimal vibration. A combing speed of 900 μm/second is used to uniformly stretched DNA molecules with minimum breaking. Around 50% of the molecules are longer than 1 Mb with an average of 2 Mb in length and 5% over 4MB.

Several other methods for stretching on a surface can be used (e.g. ACS Nano. 2015 Jan 27;9(1):809-16). Alternatively, elongation on a surface can be conducted in a flow cell including using the approach described by Petit and Carbeck (Nano. Lett. 3:1141-1146 (2003)), which show that for combing in a 20-100 uM channel a rate of fluid withdrawal of 4-5 μm/s yields a flat air-water interface which provides well aligned unidirectional polynucleotides. In addition to fluidic approaches, polynucleotides can be stretched by using an electric field (Giess et al. Nature Biotechnology 26, 317-325 (2008). Several approaches are available for elongating polynucleotides when they are not attached to a surface (e.g. Frietag et al Biomicrofluidics. 9(4):044114 (2015); Marie et al. Proc Natl Acad Sci USA. 110:4893-8 (2013)).

Extracting and Isolating DNA from a Single Cell

A number of methods have been described for isolating single cells which can be used for and extracting polynucleotides for the purpose of this invention. This includes using the device designs of WO/2012/056192, WO/2012/055415 where instead of extracting DNA and stretching in nanochannels, in the present invention the cover-glass or foil that is used to seal the micro/nanofluidic structures is coated with polyvinyl silane to enable molecular combing, by movement of fluids as described by Petit et al. Nano Letters 3:1141-1146 (2003). The gentle conditions inside the fluidic chip enables the extracted DNA to be preserved in long lengths.

Polynucleotide Repair

A polynucleotide can become damaged during extraction, storage or preparation. Nicks and adducts can form in a native double stranded genomic DNA molecule. A DNA repair solution may be introduced before or after DNA is immobilized. This can be done after DNA extraction in a gel plug. Such repair solution may contain DNA endonuclease, kinases and other DNA modifying enzymes. Such repair solution may comprise polymerases and ligases. Such repair solution may be the pre-PCR kit form New England Biolabs. The following papers are incorporated herein Karimi-Busheri F, Lee J, Tomkinson A E, Weinfeld M. Repair of DNA strand gaps and nicks containing 3′-phosphate and 5′-hydroxyl termini by purified mammalian enzymes. Nucleic Acids Res. 1998 Oct 1;26(19):4395-400. Kunkel, T A., Eckstein, F., Mildvan, A. S., Koplitz, R. M. and Loeb, L. A. (1981) Deoxynucleoside [1-thio]triphosphates prevent proofreading during in vitro DNA synthesis. Proc. Natl Acad Sci. USA, 78, 6734-6738.

Staining the Polynucleotide

Optionally, for some embodiments, to trace out the backbone of a polynucleotide DNA stains and other polynucleotide binding reagents can be used. Intercalating dyes, major groove binders, labeled non-specific DNA binding proteins cationic conjugated polymers can be bound to the DNA. Intercalating dyes can be used at various nucleobase to dye ratios. Use of multiple intercalating dye donors at a dye to base pair ratio of about 1:5-10 leads to the labeling of DNA with dye molecules (e.g., Sybr Green 1, Sytox Green, YOYO-1) sufficient to serve as donors for nucleotide additions along the growing DNA strand. Some DNA binding reagents are able to substantially cover the polynucleotide. These DNA stains can also act as FRET Partners in homogeneous or real-time sequencing. Once an intercalating dye such a YOYO-1 is added it is important to keep the DNA in the dark and to add reagents such as BME to prevent DNA nicking.

Creating Origins with Nickases and Oligos

See working examples below.

Miscellaneous Modified Nucleotides, Polymerases and Ancillary Reagents

The 3′ reversible terminating group is normally linked to the deoxyribose of the nucleotide through the oxygen atom of 3′-OH. A series of 3/-0-blocking groups have been developed including 3′-O-allyl (Ruparel et al., 2005; Wu et al., 2007), 3′-O-(2-nitrobenzyl) (Wu et al., 2007), and 3′-O-azidomethylene (Bentley et al., 2008). Reversible dye-terminators bearing either blockage group are incorporated well by a variant of archaeal 9° N DNA polymerase of hyperthermophilic Thermococcus sp. 9° N-7.Taq pol that can accept new types of reversible terminators possessing a 3′-ONH2 blocking group (dNTP-ONH2; Chen et al., 2010). The L616A Taq enzyme variants incorporated both dNTP-ONH2 and ddNTPs faithfully and efficiently.

Fluorescently labelable reversible terminators are available from Firebirdbio (http://www.firebirdbio.com/docs/FirebirdCatalog2016.pdf). Labels and oligos can be added to the TCEP cleavable disulfide nucleotide terminators. The Oxime 3′ terminator can be reverted by addition of a Nitrite. Other nucleotides can be manufactured by Jena Biosciences on a custom basis. The following polymerase reaction buffer can also be used when ss linkage is used: (20 mm Tris-HCl, pH 8.8, 10 mm mgcl2, 50 mm kcl, 0.5 mg/ml bsa, 0.01% Triton x-100).

Suitable reversible terminators that are cleavable by UV light, the Lightening

Terminators have been developed by Lasergen and are particularly suitable for increasing the speed of sequencing and for implementations of the invention in a homogeneous manner.

For the incorporation of nucleotides with bulky residues such as fluorescent labels and oligos at the 3′ end, polymerases need to have active site pockets that are compatible with such modifications. Canard and Sarfati (Gene 1994, 148, (1), have shown a of 3′-modified nucleotides, including 3′-fluothioureido-dTTP, can be incorporated by DNA polymerases including Taq DNA polymerase, Po1475 (FirebirdBio), Sequenase 2.0 Affymetrix, USA), and HIV-RT (Boehringer Mannheim). In addition Therminator™ II DNA Polymerase is a 9° N™ DNA Polymerase variant (D141A/E143A/A485L/Y409V) (NEB, USA) is able to incorporate 3′-modified nucleotides. Most current SbS methods utilize a mutagenized version of 9° N™ DNA Polymerase.

A real-time sequencing embodiment of the invention comprises a fluorescent, terminal phosphate-labeled nucleoside polyphosphates containing 3, or more, phosphates at the 5′-position of the nucleoside. Such nucleotides possessing greater than three phosphates were more effective substrates for A and B-family DNA polymerases (Kumar et al., 2005). For example labeled nucleoside penta/hexaphosphates (dN5Ps and dN6P) can be used by Phi29 DNA polymerase for incorporating thousands of bases in length, at close to native dNTP rates (Korlach et al., 2008, 2010).

The nucleotide can have dual labeled to provide dual functionality. Reversible terminators that are internally quenched have been described by Mir (WO2005040425). A first label can be a quencher modification at a terminal phosphate that can keep a base or 3′ fluorescently labeled nucleotide quenched until the nucleotide has been incorporated, it is then part of the leaving group, and once it has diffused away, fluorescence is restored. This is a way to reduce background and is particularly useful for single molecule sequencing and real-time sequencing.

Such nucleotides can comprise:

(i) Fluorophore-VT-SS-5-Aminopropargyl-ddCTP-gammahexylamino-quencher; (ii) Fluorophore-VT-SS-5-Aminoallyl-ddUTP-gammahexylamino-quencher; (ii) fluorophore-VT-SS-7-Aminopropargyl-7-Deaza-ddATP-gammahexylamino-quencher, (iv) fluorophore-VT-SS-7-Aminopropargyl-7-Deaza-ddGTP-gammahexylamino-quencher. Where SS represents a disulphide linkage which is cleavable by a reducing agent and where VT represents a linkage that enables the nucleotide to act as a virtual terminator.

The streptavidin coated nanoparticles (e.g. Quantum Dots) can be conjugated to ss-Biotin dNTPS (Perkin Elmer) in Quanatum Dot buffer for several days at 4° C., followed by 3×ultracentrifugation and removal of supernatant at 100,000 rpm on a Beckman Optima. A reducing reaction in 10 mM TCEP (or 1 or 5 or 25 mM) for 10′ minutes can break the disulphide bond to remove the nanoparticle.

Several other polymerase, nucleotide, accessory reagent combinations can be used to carry out the various embodiments of the invention as understood by an artisan skilled in the art. The extension mixture for incorporation of nucleotides comprises of 5 units of Therminator (New England Biolabs), 100 mM of each dNTP, 0.1 mg/ml glucose oxidase, 0.2 mg/mL catalase, 10% w/w glucose, 1 mM Trolox, in buffer 2 (NEB). As an alternative, the buffer can comprise or be supplemented with Ascorbate and Gallic Acid, and this is known to reduce errors in SbS reads. In addition to chemical and/or enzymatic oxygen scavenging in the flow cell/micro or nanofluidic channel, solutions can be de-gassed and oxygen can be removed from the chamber and displaced by Nitrogen; Nitrogen is used as the gas for pressure-driven flow.

Sequencing On Elongated DNA Using Fluorescent Reversible Terminators

See example below.

Super-resolution Sequencing On Elongated DNA Using Stochastic Optical Reconstruction

The above reactions and other reactions of this invention are carried out using either fluorescent labels which are switchable under certain buffer conditions or the fluorescent labels naturally blink at a rate that they can be distinguished from adjacent labels, because both are not fluorescing at the same time. One approach is to do super-resolution SbS along elongated DNA using switchable nucleotides and stochastic reconstruction or single molecule localization. Another approach is to conduct super-resolution SbS along elongated DNA using Qdot labeled nucleotides and Super-resolution optical fluctuation imaging (SOFI). The streptavidin Quantum Dots were conjugated to ss-Biotin dNTPS (Perkin Elmer) in Quantum Dot buffer for several days at 4° C., followed by 3×ultracentrifugation and removal of supernatant at 100,000 rpm on a Beckman Optima. The Qdots-dNTPs were quantitated with nanodrop spectrometer (ThermoFisher, USA). Alternatively the incubation can be carried out at 45° C. for 1 hour.Some reactions were performed in the presence of Quantum Dot streptavidin nucleotide conjugates (565 C and 655G, Quantum Dot Corporation, USA). This was incorporated into the primer and detected under TIRF microscopy in Qdot Buffer (Molecular Probes, Eugene, Oreg., USA) between the slide and a coverslip and a movie was taken to record the blinking behavior of the Qdots. The movie was then used to reconstruct a super-resolution image using methods known in the art. A reducing reaction in 10 mM TCEP (or 1 or 5 or 25 mM) for 10 minutes was followed by a further microscope examination to detect removal of the Quantum Dots.

The following polymerase reaction buffer can also be used when ss linkage is used: (20 mM Tris-HCl, pH 8.8, 10 mM MgCl2, 50 mM KCl, 0.5 mg/ml BSA, 0.01% Triton X-100).

Super-Resolution Sequencing Along Elongated DNA Using DNA PAINT

Nucleotides were tagged with oligo sequences as part 1 of a binding pair, with four distinct DNA sequences for each of the four nucleotides, each complementary to distinctly labeled DNA PAINT Imager sequence. As an alternatively to different DNA imager strands bearing different distinguishable fluorescent labels. The different imager strands, whilst bearing the same fluorescent labels can be distinguished by having different on/off binding rates. Hence their temporal signature of binding can be used to distinguish them. In addition to the imager strands bearing fluorophores, they can also be designed to carry brighter labels such as optically active nanoparticles such as semiconductor nanocrystals (201901363125).

The binding partner 1 sequence comprises a complement to the binding partner sequence 2. A list of binding pair sequences is provided in Table 1.

Biotinylated oligos (Integrated DNA Technologies) can be linked to the nucleotide or to the fluorescent label by a streptavidin-biotin interaction. Amine terminated oligos (Integrated DNA Technologies) can be linked to the nucleotide or to the fluorescent label by an Aminoallyl nucleotideN-Hydroxysuccinimide reaction

The DNA PAINT concept can be extended to other binding pairs, as long as they are able to transiently bind under reaction conditions. Again, different DNA bases can be labeled with different color imager strands or imager strands that have different on/off binding rates.

Fluorescently modified DNA oligos are purchased from Biosynthesis. Streptavidin is purchased from Invitrogen (Catalog number: S-888). Bovine serum albumin (BSA), and BSA-biotin is obtained from Sigma Aldrich (Catalog Number: A8549). Glass slides and coverslips are purchased from VWR.

Three buffers are used for sample preparation and imaging: Buffer A (10 mM Tris-HCl, 100 mM NaCl, 0.05% Tween-20, pH 7.5), buffer B (5 mM Tris-HCl, 10 mM MgCl2, 1 mM EDTA, 0.05% Tween-20, pH 8), and buffer C (1×Phosphate Buffered Saline, 500 mM NaCl, pH 8).

Fluorescence imaging is carried out on an inverted Nikon Eclipse Ti microscope (Nikon Instruments) with the Perfect Focus System, applying an objective-type TIRF configuration using a Nikon TIRF illuminator with an oil-immersion objective (CFI Apo TIRF 100×, NA 1.49, Oil). For 2D imaging an additional 1.5 magnification is used to obtain a final magnification of 2150-fold, corresponding to a pixel size of 107 nm. Three lasers are used for excitation: 488 nm (200 mW, Coherent Sapphire), 561 nm (200 mW, Coherent Sapphire) and 647 nm (300 mW, MBP Communications). The laser beam is passed through cleanup filters (ZT488/10, ZET561/10, and ZET640/20, Chroma Technology) and coupled into the microscope objective using a multi-band beam splitter (ZT488rdc/ZT561rdc/ZT640rdc, Chroma Technology). Fluorescence light is spectrally filtered with emission filters (ET525/50m, ET600/50m, and ET700/75m, Chroma Technology) and imaged on an EMCCD camera (iXon X3 DU-897, Andor Technologies).

For sample preparation, a coverslip (No. 1.5, 18×18 mm2, ≈20.17 mm thick) and a glass slide (3×1 inch2, 1 mm thick) are sandwiched together by two strips of double-sided tape to form a flow chamber with inner volume of ≈20 μL. First, 20 μL of biotin-labeled bovine albumin (1 mg/ml, dissolved in buffer A) is flown into the chamber and incubated for 2 min. The chamber is then washed using 40 μL of buffer A. 20 μL of streptavidin (0.5 mg/ml, dissolved in buffer A) is then flown through the chamber and allowed to bind for 2 min. After washing with 40 μL of buffer A and subsequently with 40 μL of buffer B, 20 μL of biotin-labeled DNA oligo template and primer (≈300 pM monomer concentration) and DNA origami drift markers (≈100 pM) in buffer B are finally flown into the chamber and incubated for 5 min. The chamber is washed using 40 pL of buffer B. 1×ThermoPol reaction buffer is flown into the chamber. This is followed by flowing in Therminator polymerase (NEB) and oligo tagged nucleotides in Therminator buffer which are allowed to react with the immobilized target polynucleotide. As the nucleotide becomes incorporated, its identity can be determined by the persistent binding of the imager strand and because of the on/off binding of the imager strand, the reactions on different target polynucleotides can be super-resolved. After imaging, the termination is reversed by photochemical cleavage of the cleavable linker and the next cycle is triggered. The buffer salt concentration can be raised to ensure effective DNA PAINT binding but this may be at the expense of nucleotide incorporation. However, salt tolerating polymerases are known including Phi29, TopoTaq and those disclosed in WO 2012173905. Hence, monovalent salt concentration of 0.65 M can be used to undertake DNA PAINT and polymerase mediated nucleotide incorporation in a homogenous reaction.

The imaging comprises 1.5 nM Cy3b-labelled imager strands for the docking strand for A nucleotide, Atto 488-labelled imager strands for the docking strand for C nucleotide, Atto 655-labelled imager strands for the docking strand for G nucleotide, and cy7-labeled imager strands for the docking strand for T nucleotide in a salt concentration in the range of buffer B at room temperature; the use of different temperatures and sequence of the oligos can require the use of different salt concentrations in the buffer. Ideally the temperature and oligo sequence is chosen so that a salt concentration suitable for the incorporation can be implemented. The CCD readout bandwidth is set to 1 MHz at 16 bit and 5.1 pre-amp gain. Imaging is performed using TIR illumination with an excitation intensity of 294 W/cm2 at 561 nm.

The DNA paint can be excited via FRET donor such as an intercalator dye, which intercalates when the duplex between the binding pairs form or a dye on binding partner 1. It is possible to obtain resolution of a few nanometers (Chemphyschem. 2014 Aug 25;15(12):2431-5).

Faster CMOS cameras are becoming available that will enable faster imaging, for example the Andor Zyla Plus allows up to 398 fps over 512×1024 with just a USB 3.0 connection, and faster over regions of interest (ROI) or a CameraLink connection. Therefore, operating with shorter docking/imager strands or at a higher temperature or lower salt concentration it is possible to gather enough information for the required resolution in short time periods; for this the laser power is preferably high, e.g. 500 mW; Camera Quantum Yield is preferably high, e.g., ˜80% and the dye brightness is preferably high. With this the acquisition time required can be reduced to a few seconds. But this can give a resolution gain of >10 fold over diffraction limit methods.

In one embodiment of the invention a novel method of imaging is implemented, using Time-delayed integration with a CCD or CMOS camera, where the sample stage is translated in synchrony with the camera read-out so that the temporal resolution is spread over many pixels. This speeds up the image acquisition as there is no delay in moving from one location on the surface to another. What results is an imaging strip, where say the first 1000 pixels in a column represent 10 seconds of imaging of one location and the next 1000 pixels represent imaging of 10 seconds of the next location. The method described in Appl Opt. 54:8632-6 (2015) can also be adapted.

When light scattering nanoparticles (e.g. gold nanoparticles) or semiconductor nanocrystals are used there is a substantial further step-up in speed, because of the brighter, near non-exhaustive optical response of these particles. Again, the camera frame rate and imager on/off rate need to be tailored to get maximum speed enhancement when using such nanoparticle labels.

An advantage of the DNA PAINT method for super-resolution imaging of the imager strand binding is that every location is always ready, there is little effect of photobleaching or dark states, and sophisticated field stops or Powell lenses are not needed to limit illumination. In addition, the effects of non-specific binding to the surface are mitigated by DNA PAINT—imager binding at non-specific sites is not persistent and once one imager has occupied a non-specific (i.e. not on the target docking) binding site it can can get bleached but remains in place blocking further binding to that location. Typically, the majority of the non-specific binding sites, which prevent resolution of the imager binding to the docking site, are occupied and bleached within the early phase of imaging, leaving the on/off binding to of the imager to the docking site to be easily observed thereafter. Hence in one embodiment, high laser power is used to bleach initial binding imagers, optionally images are not taken during this phase, and then the laser power is optionally reduced and imaging is started to capture the on-off binding to the docking sites. After the initial non-specific binding, further non-specific binding is less frequent and can be computationally filtered out by applying a threshold, for example to be considered as specific binding to the docking site, the binding to the same location must be persistent, i.e. should occur at the same site at least 5 times or more preferably at least 10 times. Typically, around 20 specific binding events to the docking site are detected.

Another means to filter out binding that is non-specific for our purpose, is that the signals must correlate with the linear strand stretched on the surface which can be done by staining the linear strand or by tracing a line through other persistent binding sites. Signals that do not fall along a line, whether they are persistant or not can be discarded.

Sequencing Along Elongated DNA Using Intercalating Dyes As FRET Donor And Photo-Chemically Cleavable Reversible Terminator Acceptors

YOYO-1 Intercalator dye is provided in the reaction mix together with ThermoPol 1 reaction buffer, Therminator polymerase and four photocleavable nucleotides (e.g. Lightning Terminators from Lasergen or equivalent nucleotides) at 65° C. for 5 to 30 minutes. Nucleotides based on Lightning Terminators can be custom synthesized and each of the nucleotides are labeled with differentiatable dyes (e.g. Cy3, Cy3.5, Cy5, Cy5.5 or Cy3B, Atto 595, tto 6555, Cy7). After the reaction, the nucleotides incorporated into the surface bound templates are detected using TIRF illumination through a high NA objective lens (1.45NA Nikon) on Nikon Ti-E microscope using Perfect Focus (PFS). Images are taken on a 512×512 ImageEM Camera (Hamamatsu). A Melles Griot 488 nM laser is fiber coupled into the TIRF attachment of the microscope. A 488 nm laser clean up filter is used along with a Longpass dichroic mirror and emission filter in the Nikon filter cube. QuadView from Photometrics is used to split the emission light by wavelength into four quadrants on the CCD camera. Following detection the fluorescent labels and terminator are cleaved using ultra-violet light exposure for 5-10 minutes. This allows the next cycle to commence.

Sequencing along elongated DNA using label on Polymerase as FRET donor and photo-chemically cleavable reversible terminator acceptors

The novel reaction is run in the presence or absence of intercalating dye using polymerase that is either directly labeled with fluorescent donors or is attached to protein (e.g., Streptavidin) which is labeled with fluorescent groups. In this embodiment, the polymerase needs to remain attached to the target polynucleotides after incorporating a base. The protein can be engineered to optimize this.

Sequencing Instrumentation

The sequencing methods of this invention have common instrumentation requirements. Basically, the instrument must be capable of imaging and exchanging reagents. The imaging requirement includes, an objective, other relay lenses, mirrors, filters and a camera or point detector. The camera includes a CCD or array CMOS detector. The point detector includes a Photomultiplier Tube (PMT) or Avalanche Photodiode (APD). Other optional aspects depending on the format of the method, an illumination source (e.g. lamp, LED or laser), translatable stage or objective, moving the sample in relation to the imager, sample mixing/agitation and temperature control.

For the single molecule implementations of the invention the illumination is preferably via the creation of an evanescent wave, via e.g. Prism-based Total Internal Reflection, Objective-based Total Internal Reflection, waveguide based TIRF, hydrogel based waveguide and bringing light into the edge of the substrate at a suitable angle. In some single molecule instruments, the effects of light scatter are mitigated by using synchronization of pulsed illumination and time-gated detection. In some embodiments dark field illumination is used.

In some embodiments the instrument also contains means for extraction of the polynucleotide from cells, nuclei, organelles, chromosome etc.

A suitable instrument for most embodiments of the invention is the Genome Analyzer IIx from Illumina; this instruments comprises Prism-based TIR, a 20× Dry Objective, a light scrambler, a 532 nm and 660 nm laser, an Infra-red laser based focusing system, an emission filter wheel, a Photometrix CoolSnap CCD camera, temperature control and a syringe pump-based system for reagent exchange. Modification of this instrument with a different lens and camera combination can enable better single molecule sequencing. The syringe-pump based reagent exchange system can also be replaced by one based on pressure-driven flow. The system can be used with a compatible Illumina flow cell or with a custom-flow cell adapted to fit the actual or modified plumbing of the instrument.

Alternatively, a motorized Nikon Ti-E microscope coupled with a laser bed (lasers dependent on choice of labels) and am EM CCD camera (e.g. Hamamatsu ImageEM) or a scientific CMOS (e.g. Hamamatsu Orca FLASH) and optionally temperature control. This is coupled with a pressure driven pump system and a specifically designed flow cell which can be manufactured for example via injection molding in Cyclic Olefin Copolymer (COC), e. g TOPAS, or PDMS or in silicon or glass using microfabrication methods. Alternatively, a manually operated flow cell can be used atop the microscope. This can be easily constructed by making a flow cell using a double sided sticky sheet, laser cut to have channels of the appropriate dimensions and sandwiched between a coverslip and a glass slide.

From cycle to cycle the flow cell can remain on the instrument/microscope, to ensure registration from frames taken at different cycles. A motorized stage with linear encoders can be used to ensure when the stage is translated during imaging of a large area, the same locations are correctly revisited cycle to cycle; Fiduciary markers, such as etchings in the flow cell can be used to validate that this is occurring correctly. Alternatively, the flow cell is removed from the instrument/microscope after each imaging round, and the incorporation reaction is done elsewhere, e.g. on a thermocycler with a flat block before it is returned to the microscope for the next round of imaging (the term imaging is used to include 2-D array or 2-D scanning detectors). In this case, it is vital to have fiduciary markings such as etchings in the flow cell or surface immobilized beads within the flow cell that can be optically detected. If the polynucleotide backbone is stained (for example by YOYO-1) their fixed position distributed locations can be used to align images from one cycle to the next.

Super-resolution microscopes such as Leica TCS SP8 STED 3× can be coupled to an optional heating mechanism and a pressure driven flow system for reagent exchange, to carry out the sequencing of this invention.

In one embodiment, the illumination mechanism described in U.S. Pat. No. 7,175,811 or Ramachandran et al (Scientific Reports 3:2133) using laser or LED illumination can be coupled with an optional heating mechanism and reagent exchange system to carry out the methods of this invention. In some embodiments a smartphone based imaging set up (ACS Nano 7:9147) can be coupled with an optional temperature control module and a reagent exchange system; principally the camera on the phone is used, but other aspects such as illumination and vibration can also be used.

Rather than using the various microscope-like components of an optical sequencing system like the GAIIx, a more integrated, monolithic device can be constructed for sequencing. Here the polynucleotide is elongated directly on the sensor array. Direct detection on a sensor array has been demonstrated for DNA hybridization to an array (Lamture et al Nucleic Acid Research 22:2121-2125 (1994)). The sensor can be time gated to reduce background fluorescence due to Rayleigh scattering which is short lived compared to the emissions from fluorescent dyes.

In one embodiment, the sensor is a CMOS detector. In some embodiments multiple colors are detected (US20090194799). In some embodiments the detector is a Foveon detector (e.g. U.S. Pat. No. 6,727,521). The sensor array can be an array of triple-junction diodes (U.S. Pat. No. 9,105,537). In some embodiments the four different labels are not coded by wavelength of emission. In some embodiments the four different labels coded by fluorescence lifetime.

It is advantageous to use a single wavelength as a light source and not have to use filters, both for the simplicity of the set-up and because there is inevitably some loss of light when filters are used. In some embodiments the four different labels are coded by repetitive on-off hybridization kinetics; four different binding pairs with different association-dissociation constants are used. In some embodiments the nucleotides are coded by fluorescence intensity. The nucleotides can be fluorescent intensity coded by having different number of non-self quenching fluors attached. The individual fluorophores typically need to be well separated in order not to quench and a rigid linker or a DNA nanostructure where they are held in place at a suitable distance is a good way to achieve this. One alternative embodiment for coding by fluorescence intensity is to use dye variants that have similar emission spectra but their quantum yield or other measureable optical character differs, for example Cy3B (558/572)is substantially brighter (Quantum yield 0.67) than Cy3 (550/570) (Quantum yield 0.15) but have similar absorption/emission spectra. A 532 nm laser can be used to excite both dyes. Other dyes that can be used include Cy3.5 (591/604) which while has an up shifted excitation and emission spectra, will nonetheless be excited by the 532 nm laser but will emit weaker than Cy3 even though both have similar quantum yields, Cy3.5 is being excited by a sub-optimal wavelength. Atto 532 (532/553) has a quantum yield of 0.9 and would be expected to be the brightest as the 532 nm laser hits it at its sweet spot.

Current optical sequencing methods require an image processing step in which the sequence signals are extracted from the images. This usually involves extracting the relevant signals from each frame of the image. In one embodiment, an alternative is to capture signals from all pixels, vertically through all cycles and use an algorithm to compute the sequence. One advantage of this approach is that when the trajectory of signals is viewed vertically through the cycles, it is easy to filter out non-specific or background signals, they do not usually occur at the same location through the cycles, whereas the real incorporations do. It is also easy to determine which signals belong to a particular elongated molecule as they can be traced by a straight line through a series of pixels. In some embodiments the size of a single pixels is matched (via magnification) to the size of point source.

Lipid Passivation

Surfaces can be passivated using Lipids as described in doi: 10.1021/n1204535h, incorporated herein in its entirety by reference. For the creation of lipid bilayers (LBLs) on the surface of nanofluidic channels we used zwitterionic POPC (1-palmitoyl-2-oleoyl-sn-glycero-3-phosphocholine) lipids with 1% Lissamine™ rhodamine B 1,2-dihexadecanoyl-sn-glycero-3-phosphoethanolamine, triethylammonium salt (rhodamine-DHPE) lipids added to enable observation of the LBL formation with fluorescence microscopy. Prior to each coating procedure, lipid vesicles of approximately 70 nm diameter were created by extrusion (see ESI). The extruded vesicle solution was flushed through one of the microchannels of the fluidic system. Subsequently, the lipid vesicles settle down on the surface, rupture and form patches of LBL that connect within a few minutes to a continuous LBL, coating the entire microchannel. The LBL is subsequently allowed to spread spontaneously into the nanochannels while the flow of lipid vesicles is sustained in the coated microchannel to ensure a steady supply of vesicles. During the coating process a counter flow (˜80 μm/s) through the nanochannels is imposed into the coated microchannel to avoid any debris or vesicles in the nanochannels. An alternative slightly quicker method was also tested involving flushing lipid vesicles from the LBL-coated microchannel through the nanochannels results in deposition and rupture of lipid vesicles inside the nanochannels. However, with this method care needs to be taken to prevent vesicles and other residues from getting deposited and potentially blocking the nanochannels.

Transposition on Long Genomic DNA

Each reaction mixture contains (in a final volume of 20 μL) 1 ng of high-molecular-weight genomic DNA, the sequence to be inserted (e.g. Ilumina Nextera FC-121-1031, FC-121-1030), 10 μl of 2×Nextera Tagment DNA (TD) buffer from the Nextera DNA Sample Preparation kit (Illumina, FC-121-1031) and 8 μl of water. 2.5 pmol of each transposome complex is added and allowed to mix. This transposition mix is incubated at 55° C. for 10 min in a thermocycler with a heated lid. The Tn5 transposase cuts the sample DNA and adds the insert sequence at either end of each fragment and holds the fragments together. Transposition is stopped by adding 20 μl of 40 mM EDTA (pH 8.0) to each reaction and incubating at 37° C. for 15 min. The DNA is stretched out on to the surface. To dissociate Tn5 from the transposed DNA, 2 μl of 1% SDS is added, gently mixed and incubated at 55° C. for 15 min. After a 5-min incubation, heated the flow cell is heated at 1° C./s to 55° C.

Illumina wash/amplification buffer is injected into the flow cell. PEG 8000 can increase reaction efficiency. After stretching, the DNA is denatured with alkali (0.5M NaOH). The denatured DNA is optionally covered with polyacrylamide gel. Then primers are added to bind to the inserted sequence. The flow cell is then placed on a flat-block PCR machine (G-Storm) and PCR was carried out for 10-20 Cycles. Optionally the primers contain crosslinking modifications. Tn5 protein is available from Epicenter or the plasmid from Addgene (ID: 60240).

Indexing and Multiplexing genomic DNA Samples

A different index sequence is included in the above reaction for different samples (e.g. Nextera Index Kit, FC-121-1012, FC-121-1011). The samples are then pooled 20 μl from each well into a plastic container and gently rocked for 5 min at 2 r.p.m. to mix well. The 25 pg/μl pool is then diluted to 1 pg/μl in 1× TE buffer (10 mM Tris-HCl, 1 mM EDTA, pH 8.0) in a PCR strip tube. 10-50 pg of the diluted pool is then added to the flow cell pre-washed with 200 ng of BSA and the strands are stretched by containing (New England BioLabs).

Epi-Marking Reagents and Labelling Methods

Epigenomic or epignenetic modifications (Epi-Marks) on polynucleotides can be detected using the methods of the invention. Focus here is on binding to methyl groups on genomic DNA, which in humans occurs in the form of 5-MethylCytosine and usually in the context of the CpG motif. However, the same principles can be applied other modifications such a hydroxyl methyl C as well as DNA damage of various kinds. Antibodies raised against different epigenomic modifications and sites of DNA damage can be labeled by standard antibody labeling kits such as lightning link and the labeled antibodies can be bound to the polynucleotides in PBS buffer. Other reagents such as methyl binding proteins can be labeled and applied to polynucleotides in the same way.

### EXAMPLES

The following examples are described as manual operations but can easily automated using tubing, a syringe pump and valves under computer control through LabView (National Instruments), for example.

### Example 1. Sequencing a Double Stranded Polynucleotide (e.g. Genomic DNA)

Step 1—Extracting Long Lengths of Genomic DNA

NA12878 cells are grown in culture and harvested. They are mixed with low-melting temperature agarose heated to 60° C. The mixture is poured into a gel mould (e.g. purchased from Bio-Rad) and is allowed to set into a gel plug, to give approximately 4×10⁷ cells (this number can be higher or lower depending on the desired density. The cells in the gel plug are lysed by bathing the plug in a solution containing Proteinase K. The gel plugs are gently washed in TE buffer (e.g. in a 15 ml falcon tube filled with wash buffer but leaving a small bubble to aid in the mixing, and placing on a tube rotator). The plug is placed in a trough with around 1.6 ml volume and DNA is extracted by using agarase enzyme to digest the DNA. The FiberPrep kit (Genomic Vision, France) and associated protocols can be used to carry out this step.

Step 2—Stretching Molecules on a Surface

The final part of step 1 renders the extracted polynucleotides in a trough in a 0.5M MES pH 5.5 solution. The substrate cover glass, coated with vinyl silane (e.g. CombiSlips from Genomic Vision) is dipped into the trough and allowed to incubate for 1-10 minutes (depending on the density of polynucleotides required). The cover glass is then slowly pulled out, using a mechanical puller, such as a syringe pump with a clip attached to grasp the cover glass (alternatively the FiberComb system from Genomic Vision can be used). The DNA on the cover glass is crosslinked to the surface using an energy of 10,000 microJoules using a crosslinker (Stratagene, USA). If the process is carried out carefully, it easily results in High Molecular Weight (HMW) polynucleotides with an average length of 200-300 Kb elongated on the surface, with molecules greater than 1 Mb, and even in the 10 Mb range amongst the population of polynucleotides. With greater care and optimization the average length can be shifted to the megabase range (see mega-base range combing section above).

As an alternative pre-extracted DNA (e.g. Human Male Genomic DNA from Novagen cat no 70572-3 or Promega) can be used and comprises a good proportion of genomic molecules of greater than 50 Kb. Here a concentration of approximately 0.2-0.5 ng/μL, with dipping for approximately 5 minutes is sufficient to provide a density of molecules where a high fraction can be individually resolved using diffraction limited imaging.

Step 3—Making Flow Cell

The cover glass is pressed onto a flow cell gasket fashioned from double sided sticky 3M sheet which has already been attached to a glass slide. The gasket (with both sides of the protective layer on the double-sided sticky sheet on) is fashioned, using a laser cutter, to produce one or more flow channels. The length of the flow channel is longer than the length of the cover-glass, so that when the cover-glass is placed at the center of the flow channel, the portions of the channel one at each end that are not covered by the cover glass can be used as inlets and outlet for dispensing fluids into and out of the flow channel, such fluids passing atop the elongated polynucleotides on the vinyl silane surface). The fluids can be flowed through the channel by using safety swab sticks (Johnsons, USA at one end to create suction as fluid is pipetted in at the other end. The channel is pre-wetted with Phosphate Buffered Saline-Tween and Phosphate Buffered Saline (PBS-washes).

Alternatively, the cover glass can be sealed onto the channels of the Sticky slide system from Ibidi (Germany). Another alternative is to stretch the DNA in a pre-made fluidic device in which an internal surface comprises vinyl silane. The DNA can also be extracted within the fluidic device, by depositing the gel plug into the inlet of the device or by directly capturing cells within the device and extracting using the methods described for cells and chromosomes in doi: 10.1073/pnas.1804194115, doi: 10.1039/c81c00169c; doi: 10.1038/s41598-017-10704-4, doi: 10.1073/pnas.1214570110, doi: 10.1039/c01c00603c which are incorporated herein in their entirety. Other surfaces on which DNA can be stretched include APTES, Zeonex and PMMA surfaces.

Step 4—Passivation

Optionally a blocking buffer such as Blockaid (Invitrogen, USA) is flowed in and incubated for ˜5 minutes. This is followed by Phosphate Buffered Saline-Tween (PBS-T) washes. This step can optionally be carried out after step 6.

Step 5—Creating Nicks on the DNA

After hydrating the stretched DNA with. It is pre-conditioned with DNAse1 buffer. The DNAse1 reaction is undertaken using 5 units DNAse 1 enzyme in DNAase1 buffer (Roche) in a 20 ul reaction the reaction is incubated at room temperature for 10 minutes and allowed to incubate for 10 minutes (or longer or shorter depending on the frequency of nicking required; the concentration of the DNAse1 is also adjusted accordingly) at room temperature. After nicking the DNAse1 is washed out by pipetting wash buffer (PBST-washes) into the inlet at one end of the channel and using the safety swab stick at the other end (using a pipette tip to dispense into the inlet and a 1 ml luer syringe at the outlet in the case of the Ibidi flow channel). Alternatively, nicks can be made using the nicking endonuclease, Nt.CViPII (NEB). In this case the flow cell is pre-conditioned with NEB CutSmart buffer supplemented with ˜0.1% Triton X. The reaction is carried out at room temperature (or at 37° C.), using 2.5 Unis of the enzyme in the CutSmart/TritonX buffer in a 30 ul reaction for 10 minutes or longer depending on the density of nicks required; the concentration of the Nt.CViPII is also adjusted accordingly. Following the reaction, the flow cell is washed with PBS-washes. It should be noted that an exonuclease activity is present with this enzyme The nicking time and temperature can be varied depending on the density of nick sites desired.

Step 6—Adding Nucleotide Mix

The flow cell is pre-conditioned with Illumina High-Salt Buffer and Incorporation buffer. A mixture of nucleotides with polymerase (Illumina incorporation mix, for the GAIIx, for example) are pipetted at the inlet and then flowed through into the channel. The reaction is allowed to proceed at the appropriate temperature (60° C.; or within the 55-65° C. range) for 10-15 minutes on a Thermomixer flat block (Eppendorf, USA), replenishing with reagent, if the channel starts to become dry. Alternatively, Lasergen nucleotides and Therminator polymerase or FireBirdBio nucleotides and Proprietary Taq-based polymerase variant can be used together with the attendant protocols. In the case where binding partners are used for DNA PAINT, the nucleotides are tagged with an oligonucleotide rather than a fluorescent label and detection is achieved by the transitory binding of fluorescently labeled oligonucleotides (Imagers) that are complementary to the oligonucleotide tags, as described in United States Patent Application 20180327829, which is incorporated herein in its entirety.

Step 7—Imaging-Determining the Location and Identity of Nucleotides Incorporated

The flow channel is placed on an inverted microscope (e.g. Nikon Ti-E) equipped with Perfect Focus, TIRF attachment, and TIRF Objective, lasers (red and green) and a Hamamatsu or Andor EMCCD camera. Illumina Imaging buffer is added (which can be supplemented or replaced a buffer containing Beta Mercaptoethanol, Enzymatic redox system, and/or Ascorbate and Gallic Acid) Fluorophores are detected along lines, indicating that incorporation has occurred on elongated polynucleotides (otherwise the signals would be random only). The location of each fluorescent point signal is detected, recording the pixel locations whereupon the fluorescence from the nucleotide labels is projected. The identity of the incorporate nucleotide is determined by using filters to determine which of the nucleotides have been incorporated. The fluorophores, may be detected across multiple filters and in this case the emission signature of each flurophore across the filter set is used to determine the identity of the fluorophore and hence the nucleotide. Optionally, if the flow cell is made with more than one channel, one of the channels can be stained with YOYO-1 intercalating dye, for checking the density of polynucleotides and quality of the polynucleotide elongation (using Intensilight and Nikon B-2A filter or 488 nm laser illumination and a 488 laser filter set from Chroma). Four images are taken, one tailored for each of the four fluorescent wavelengths.

When the single molecule localization technique is used to pinpoint the location of fluorescent signals, a number of measures need to be implemented to get the highest resolution. The images have to be processed using single molecule localization algorithms (e.g. Thunderstorm, Picasso software). Also, a sufficient number of photons need to be collected and drift has to be corrected. The drift correction can be done after the fact, using tools included in the localization software. This can be aided by the provision of fiducial markers. Suitable fiducial markers include, gold nanoparticles (Cytodiagnostics), Fluospheres (Thermofisher) and Nanodiamonds (Adamas), when their brightness matched to the brightness of the fluorescent labels. Drift can also be corrected without fiducials, using the locations of the template molecules themselves (e.g. the line patterns generated by signals along the length of the polynucleotide strands). Drift correction can also be done during the course of imaging (Coelho et al Biorxiv http://doi.org/10.1101/487728).

Step 8—Imaging-Moving to Other Locations

The cover glass (via a glass slide) which has been mounted onto a translation is translated with respect to the objective lens (hence the CCD) so that a separate location can be imaged. The imaging is done at a multiple of other locations so that genomic molecules or parts of molecules rendered at different locations (outside the field of view of the CCD at its first position) can be imaged and the incorporated nucleotides detected. The image data from each location is stored in computer memory or on the cloud e.g. Amazon Web Services (AWS).

Step 9—Reversing Termination

Termination is reversed by first washing with Illumina Cleavage buffer and then adding Illumina Cleavage solution (or in the case of using Lasergen chemistry, shining UV light onto the surface; or in the case of using FireBirdbio chemistry TCEP and Nitrite can be added). This is followed by PBS-washes. Optionally an image is taken to ensure cleavage has taken place.

Step 10—Repeating Until One Sequence Read Coalesces with Another

The incorporation and reversal is repeated (steps 6-9) until a sufficient number is done to allow coalescence of reads from one site to an adjacent site of initiation in the desired threshold number of cases. The number of cycles is determined by taking into account the degree of stretching of the polynucleotide and the distance between the start sites. The number of cycles to be conducted can be predetermined and may be between the 118 ange 5 and 900 cycles. Optionally steps 5-9 are repeated.

Step 11—Data Processing

The collected images are image processed by applying algorithms that take into account the location of the signals on the sensor, for the imaging channel for each of the fluorescent wavelengths. Each of the locations is tracked over multiple images and for each of the wavelength channels to discern if a nucleotide incorporation is occurring at the location and the identity of the incorporated nucleotide, all through the multiple cycles of the sequencing reaction. The algorithms use this information to find which signals are occurring over a line that traces out an elongated polynucleotide make base calls at each location, for each of the sequencing cycles. This results in spatially distinct reads along the length of a polynucleotide. An algorithm is then used to re-construct a longer range polynucleotide sequence either by coalescence of reads or integration of spatial read information from other copies of the polynucleotides.

### Example 2. SbS from Oligos Annealed on Single Stranded Polynucleotides

In one embodiment an RNA polynucleotide or denatured DNA polynucleotide is sequenced. Steps 1, 2, 3 and are 4 common with example 1 above, but instead of step 5 (nicking) denaturation is done instead and oligos are added:

Step 5—Denaturation of dsDNA

ds DNA was denatured by flushing alkali (0.5M NaOH) through the flow cell and incubating for approximately 20 minutes at room temperature. This is followed by PBS-washes. (Alternatively, incubation with 1M HCL for 1 hour followed by water washes and a 5 minute TE wash can be done).

Step 6—Adding Oligos

The flow cell is pre-conditioned with hybridization buffer (2×SSC, 50% Formamide, 33% Blockaid, 0.1% SDS or 3M TMACL, 50 mM Tris Cl ph8, 0.4% BME, 0.05% Tween 20).

800 nM oligos are bound to the elongated denatured polynucleotides. The length of the oligo primer can range from typically range from 10 to 30 nucleotides and the reaction temperature depends on the Tm of the primer. The sequence of the oligo determines where along the strand it will bind, lengths ranging from 14 nt and above can be used to selectively sequence chosen parts of the polynucleotide This is followed by steps 7-11 above.

This is followed by steps 7 and 8 before optional step 9-10 below and, step 11.

Step 9—Removing Oligos

Oligos are removed by flushing alkali (0.5M NaOH) through the flow cell and incubating for approximately 5-20 minutes at room temperature (alternatively, heating, formamide, 1M HCL, 7M Urea, can be used). This is followed by PBS-washes. Optionally an image is taken to ensure sufficient oligo removal has taken place.

Step 10—Adding the Next Set of Oligos

The next set of oligos are added and steps 6-9 are repeated until the whole of the polynucleotide has been sequenced.

Step 11—Data Processing

The collected images are image processed by applying algorithms that take into account the location of the signals on the sensor. Each locations is tracked over multiple images and for each of the wavelength channels to discern if an oligo hybridization has occurred at the location, all through the multiple cycles of hybridization. The algorithms use this information to find which signals are occurring over a line that traces out an elongated polynucleotide, determines the presence and absence of oligo binding at each location, for each of the hybridization cycles. This results in spatially distinct reads along the length of a polynucleotide. An algorithm is then used to re-construct a longer range polynucleotide sequence either by coalescence of reads or integration of spatial read information from other copies of the polynucleotides.

### Example 3. Methylation Labelling on Single Stranded Polynucleotide

Steps 1, 2, 3 and are 4 common with example 1 and step 5 is common with example 2. Step 11 is common with example 1 but epi-mark information is processed rather than sequencing information.

Step 6—Binding of Anti-Methyl C Antibody.

The flow cell is flushed with PBS-washes and the anti-methyl antibody 3D3 clone (Diagenode) in Phosphate Buffered Saline is added and incubated for one hour. Optionally the proteins or antibodies can be fixed to the DNA using 2% Formaldehyde (Thermofisher).

Step 7—Imaging-Determining the Location of Epi-Marks

The flow channel is placed on an inverted microscope (e.g. Nikon Ti-E) equipped with Perfect Focus, TIRF attachment, and TIRF Objective, lasers and a Hamamatsu or Andor EMCCD camera. Imaging buffer is added (which can be supplemented or replaced by a buffer containing Beta-Mercaptoethanol, Enzymatic redox system, and/or Ascorbate and Gallic Acid). Fluorophores are detected along lines, indicating that binding has occurred along stretched DNA strands. Optionally, if the flow cell is made with more than one channel, one of the channels can be stained with YOYO-1 intercalating dye, for checking the density of polynucleotides and quality of the polynucleotide elongation (using Intensilight or 488 nm laser illumination).

Step 8—Imaging-Moving to Other Locations

The cover glass which has been mounted onto a translation stage (via a glass slide) is translated with respect to the objective lens (hence the CCD) so that a separate location can be imaged. The imaging is done at a multiple of other locations so that genomic molecules or parts of molecules rendered at different locations (outside the field of view of the CCD at its first position) can be imaged and the methyl binding sites detected. The image data from each location is stored in computer memory or in an Amazon cloud cluster.

Step 9—Stripping of f Anti-Methyl C Antibody

Typically, the epi-analysis is done before sequencing, therefore optionally the bound antibodies are removed from the polynucleotide before sequencing commences. This can be done by flowing through a high salt buffer and SDS and checking by imaging that removal has occurred. If it is evident that more than a negligible amount of antibody remains, then harsher treatments such as the chaotrophic salt, GuCL can be flowed through to remove what remains.

Step 12—Data Correlation

After sequencing data has been obtained the result of locational methylation analysis is correlated with locational DNA analysis.

### Example 4. Methylation labelling on double stranded polynucleotide

Steps 1, 2, 3 and are 4 common with example 1 and step 5 is common with example 2. Step 7 and 8 is common with example 4. Step 11 is common with example 1 but epi-mark information is processed rather than sequencing information. Step 12 is the same as Example 4.

Step 6—Binding of Methyl Binding Domain (NBD) protein

The flow cell is flushed with Phosphate Buffered Saline and labeled MBD1 is bound. Optionally the proteins or antibodies can be fixed to the DNA using 2% Formaldehyde.

Step 9 Stripping off MBD

Typically, the epi-analysis is done before sequencing, therefore optionally the bound proteins are removed from the polynucletide before sequencing commences. This can be done by flowing through a high salt buffer and SDS and checking by imaging that removal has occurred. If it is evident that more than a negligible amount of antibody remains, then harsher treatments such as the chaotrophic salt, GuCL can be flowed through.

### Example 5: Amplifting and Sequencing Segments of the Genome in their Long-Range Context

Step 1:
Insert primer binding sites (PBS) along the length of the genomic DNA according to the Tagmentation protocol described above
Step 2:
Stretch the DNA on a glass surface within a flow cell e.g. an Illumina flow cell compatible with Illumina Genome Analyzer IIx or a similar obtained from vendor such as Dolomite (UK).
Step 3:
Coat the stretched DNA by polymerizing with Acrylamide/bis (30% 37.5:1; Bio-Rad), N,N,N′,N′-tetramethylethylene-diamine (TEMED) (Bio-Rad), Ammonium
Step 4:
Carry out the Polymerase chain reaction (PCR) by adding primers, nucleotides and polymerase to the flow cell on a flat block PCR machine (G-Storm), culminating in a denaturation step, with optional addition of 0.5M NaOH for further denaturation.
Step 5:
Add sequencing primer (complementary to the primer binding site added by tagmentation) to the amplified DNA spatially localized within the gel followed by Illumina polymerase and fluorescently labeled reversible terminator mixture.
Step 6:
Run Genome Analyzer IIX, comprising incorporation, imaging and cleavage steps.
Step 7:
Process images to obtain sequencing reads for each of the spatially localised segmental amplicons and stitch them together, subtract the sequence of the inserted primer binding site to obtain the long-range sequence of the genomic DNA
The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. Those skilled in the art will recognize, or be able to ascertain using no more than routine experimentation, many equivalents to the specific embodiments of the invention described herein. Such equivalents are intended to be encompassed by the following claims.

What is claimed:

1. A method of sequencing a single, elongated target polynucleotide molecule comprising:
(a) seeding a plurality of separately resolvable origins of polynucleotide synthesis along the single, elongated target polynucleotide molecule;
(b) contacting the target polynucleotide molecule with a polymerase and labeled nucleotides;
(c) incorporating a labeled nucleotide, using the polymerase, into a plurality of sequence fragments complementary to the target polynucleotide molecule in a template-directed reaction originating from the origins of polynucleotide synthesis;
(d) detecting and storing in computer memory respective identity and positions of the labeled nucleotide incorporated into each of the plurality of sequence fragments; and
(e) repeating steps (c) and (d) until a threshold fraction of adjacent sequence fragments merge and result in continuous sequence reads spanning two or more adjacent sequence fragments.

2. The method of claim 1, wherein (the threshold fraction is low and) gaps remain, such gaps are filled by other polynucleotides that have been sequenced, wherein the same gaps are not present.

3. The method of claim 1, wherein (the threshold fraction is high and) negligible number of gaps remain, a substantially complete genome sequence is obtained without sequencing of other polynucleotides.

4. The method of claim 1, wherein step (b) comprises simultaneously contacting the target polynucleotide molecule with a polymerase and four types of differently labeled nucleotides comprising A, C, G, and T/U.

5. The method of any one of claim 1 or 4, wherein the nucleotides are reversible terminators and identifying the identity and positions of the labelled nucleotide is via detecting a signal from the labelled nucleotide and repeating of step b or c is preceded by reversing the termination.

6. The method of claim 1, wherein step (b) comprises contacting the target polynucleotide molecule with a polymerase and a single type of labeled nucleotide selected from the group consisting of A, C, G, and T/U.

7. The method of claim 6, wherein the incorporation of the nucleotide is detected by detecting a spatially resolvable signal.

8. The method of claim 7, wherein the spatially resolvable signal is due to one or more labels on the polymerase or nucleotide.

9. The method of claim 1, wherein the single target polynucleotide is a chromosome.

10. The method of claim 1, wherein the single target polynucleotide is about 10², 10³, 10⁴, 10⁵, 10⁶, 10⁷, 10⁸ , 10⁹ bases in length.

11. The method of claim 1, wherein the single target polynucleotide is single stranded.

12. The method of claim 1, wherein the single target polynucleotide is double stranded.

13. The method of claim 1, further comprising extracting the single target polynucleotide molecule from a cell, organelle, chromosome, virus, exosome or body fluid or substance with minimal degradation.

14. The method of claim 1, wherein the target polynucleotide molecule is stretched.

15. The method of claim 1, wherein the target polynucleotide molecule is immobilized on a surface.

16. The method of claim 1, wherein the target polynucleotide molecule is disposed in a gel.

17. The method of claim 1, wherein the target polynucleotide molecule is disposed in a micro- or nano-fluidic channel.

18. The method of claim 1, wherein the target polynucleotide molecule is substantially intact.

19. The method of claim 1, wherein the merging of the adjacent sequence fragments comprises an overlap of at least 5 bases between the adjacent sequence fragments.

20. The method of claim 1, wherein the merging of the adjacent sequence fragments is determined by the relative positions of the adjacent sequence fragments abutting and/or overlapping.

21. The method of claim 1, wherein the merging of the adjacent sequence fragments is determined by the sequences of the adjacent sequence fragments overlapping.

22. The method of claim 1, wherein the adjacent separately resolvable origins of polynucleotide are separated by about 10, 50, 100, 250, 500, 750, 1,000, 5,000, or 10,000 bases.

23. The method of claim 1, wherein the adjacent separately resolvable origins of polynucleotide comprise natural sequences of the target polynucleotide.

24. The method of claim 1, wherein the adjacent separately resolvable origins of polynucleotide comprise (the 3′ of) synthetic (origin-related) sequences annealed to the target polynucleotide.

25. The method of claim 1, wherein the adjacent separately resolvable origins of polynucleotide synthesis comprise synthetic (origin-related) sequences incorporated/inserted into the target polynucleotide (e.g. via transposase).

26. The method of claim 25, wherein the inserted sequence includes an indexing sequence adjacent to the origin-related sequence.

27. The method of claim 1, further comprising:
(f) ascertaining and storing the positions of the first and second locations in a computer memory;
(g) storing the position and identity of the differently labeled nucleotides incorporated into the first sequence fragment and the second sequence fragment in step (e); and
(h) ascertaining when the first and second sequence fragments coalesce and assembling the stored identity of the differently labeled nucleotides, thereby sequencing the single target polynucleotide.

28. The method of claim 24, further comprising computationally trimming an overlapping segment of adjacent sequence fragments.

29. The method of claim 1, further comprising:
(f) seeding a second plurality of separately resolvable origins of polynucleotide synthesis along the single, elongated target polynucleotide molecule;
(g) contacting the target polynucleotide molecule with the polymerase and labelled nucleotides;
(h) incorporating the labelled nucleotides, using the polymerase, into a second plurality of sequence fragments complementary to the target polynucleotide molecule, in a template-directed reaction and originating from the second plurality of separately resolvable origins of polynucleotide synthesis;
(i) identifying and storing the identity and positions of the labelled nucleotides incorporated into each of the second plurality of sequence fragments, thereby determining the sequences and relative positions of the second plurality of sequence fragments;
(j) repeating steps (g), (h) and (i) until a second threshold fraction of adjacent sequence fragments merge and result in continuous sequence reads spanning two or more adjacent sequence fragments; and
(k) combining the sequence reads from steps (e) and (j), thereby sequencing the target polynucleotide molecule.

30. The method of claim 1, wherein the sequence is determined without using another copy of the target polynucleotide molecule or reference sequence for the target polynucleotide molecule.

31. The method of claim 1, further comprising computationally trimming an overlapping segment of adjacent sequence fragments.

32. The method of claim 1, further comprising: (f) repeating steps (c) and (d) until a threshold fraction of adjacent sequence fragments overlap and result in redundant sequence reads spanning two or more adjacent sequence fragments.

33. The method of claim 31, further comprising: (g) identifying any inconsistencies in the redundant sequence reads as potential sequencing errors or ambiguities.

34. The method of claim 1, further comprising:
(f) degrading at least a fraction of the plurality of sequence fragments; and
(g) repeating steps (c) and (d), thereby resequencing the plurality of sequence fragments.

35. The method of claim 34, wherein a 3′ to 5′ exonuclease is used to degrade the fraction of the plurality of sequence fragments and optionally the degradation stops at the origin.

36. The method of claim 34, wherein the differently labeled nucleotides are degradable nucleotides

37. The method of claim 36, wherein the degradable nucleotides are 5′ amide modified nucleotides and are cleaved by acid.

38. The method of claim 36, wherein the degradable nucleotides are RNA and are cleaved by an RNAse and/or alkali.

39. The method of claim 36, wherein the degradable nucleotides are RNA and further comprising the steps of:
(f) degrading at least one of the degradable nucleotides to leave an abasic site or nick; and
(g) repeating step (c) using the abasic site or nick as an origin of polynucleotide synthesis.

40. A method of haplotype resolved sequencing comprising:
sequencing a first target polynucleotide spanning a haplotype of a diploid genome using the method of claim 1;
sequencing a second target polynucleotide spanning a haplotype of the diploid genome using the method of claim 1,
wherein the first and second target polynucleotides are from different
homologous chromosomes (chromosome homologues); and
thereby determining the haplotypes on the first and second target polynucleotides.

41. A method of haplotype resolved sequencing of a polyploid genome comprising:
sequencing a first target polynucleotide spanning a first haplotype of a polyploid genome using the method of claim 1;
sequencing a second target polynucleotide spanning a second haplotype of the polyploid genome using the method of claim 1;
sequencing further target polynucleotide spanning further haplotypes of the polyploid genome using the method of claim 1
wherein the first and second and further target polynucleotides are from different homologous chromosomes (chromosome homologs); and thereby determining the first, second, and further haplotypes of the polyploid genome.

42. A method of obtaining a long-contiguous sequencing read comprising
obtaining a first short read;
obtaining a second short read adjacent to the first read;
obtaining further short reads adjacent to the first and/or second short read; and
stitching at least two short reads together to obtain a contiguous long read.

43. The method of claim 42 wherein some of the reads are obtained from different polynucleotide molecules

44. The method of claim 43 wherein some of the reads from different polynucleotides overlap sufficiently for the sequence of the different molecules to be aligned.

45. The method of any of the previous claims wherein the reads are generated by identifying and storing the identity and positions of the labeled nucleotide incorporated into each of the plurality of sequence fragments by using super-resolution/single molecule localization.

46. The method of claim 45, wherein the super-resolution/localization is virtual, and comprises using a reference sequence to assign unresolved signals from multiple origins to the correct origins.

47. The method of claim 45, wherein the super-resolution single molecule localization is done via Stochastic Optical Reconstruction Microscopy (STORM), Super-resolution optical fluctuation imaging (SOFI), Microscopy or Points Accumulation for Imaging in Nanoscale Topography (PAINT) or other high resolution or nanometric localization method.

48. The method of claim 47, wherein PAINT comprises DNA PAINT.

49. The method of any one of claims 1-46, wherein the segments of the elongated polynucleotide that are sequenced are amplified in situ before sequencing.

50. The method of claim 49, wherein the amplification occurs using the origin-related sequences inserted into the target polynucleotide as primer binding sites or promoters.

51. The method of any one of the previous claims, wherein the target polynucleotides are contacted with a gel or matrix layer.

52. The method of claim 1, wherein the origins are seeded, in close to random manner by incubating double stranded DNA with Nt.CViPII or derivatives.

53. The method of claim 1, wherein sequencing is combined with analysis of epi-marks (e.g. methylation) by the labeling of epi-marks orthogonally to sequencing.

54. The method of claim 53 wherein the epi-marks are labeled such that they can be super-resolved or subjected to single molecule localization (e.g. by DNA PAINT).

55. A method of sequencing a target polynucleotide molecule comprising:
(a) seeding a plurality of separately resolvable origins of polynucleotide synthesis along each of a plurality of copies of the target polynucleotide molecule;
(b) contacting the plurality of copies with a polymerase and four types of differently labelled nucleotides simultaneously;
(c) incorporating the differently labelled nucleotides, using the polymerase, into a plurality of sequence fragments complementary to the target polynucleotide molecule and originating from the origins of polynucleotide synthesis;
(d) identifying and storing the identity and positions of the differently labelled nucleotides incorporated into each of the plurality of sequence fragments, thereby determining the sequences and relative positions of the plurality of sequence fragments;
(e) repeating steps (c) and (d) until a threshold number of nucleotides are sequenced; and
(f) assembling the plurality of sequence fragments, thereby determining the sequence of the elongated, target polynucleotide molecule.

56. A method of sequencing a single, elongated target polynucleotide molecule comprising:
(a) seeding a plurality of separately resolvable origins of polynucleotide synthesis along the target polynucleotide molecule;
(b) contacting the target polynucleotide molecule with a polymerase and four types of differently labelled nucleotides simultaneously;
(c) incorporating the differently labelled nucleotides, using the polymerase, into a plurality of sequence fragments complementary to the target polynucleotide molecule and originating from the origins of polynucleotide synthesis;
(d) identifying and storing the identity and positions of the differently labelled nucleotides incorporated into each of the plurality of sequence fragments, thereby determining the sequences and relative positions of the plurality of sequence fragments; and
(e) repeating steps (c) and (d) until a threshold number of nucleotides are sequenced; and
(f) comparing the sequences and relative positions of the plurality of sequence fragments to a reference sequence for the target polynucleotide molecule, thereby ascertaining any differences in sequence and/or structure between the target and the reference sequence.
